(19) United States
(12) Patent Application Publication
(10) Pub. No.: US 2017/0131237 A1
(43) Pub. Date: May 11, 2017

(54) NANOGAP ELECTRODE DEVICES AND SYSTEMS AND METHODS FOR FORMING THE SAME

(71) Applicant: Quantum Biosystems Inc., Tokyo (JP)

(72) Inventor: Shuji Ikeda, Tokyo (JP)

(21) Appl. No.: 15/336,515

(22) Filed: Oct. 27, 2016

Related U.S. Application Data
(63) Continuation of application No. PCT/JP2015/063403, filed on Apr. 28, 2015.

(30) Foreign Application Priority Data
Apr. 28, 2014 (JP) 2014-093079
May 2, 2014 (JP) 2014-095163

(51) Int. Cl. G01N 27/447; G01N 33/487

(52) U.S. Cl. CPC G01N 27/44791; G01N 33/48721

(57) ABSTRACT

The present disclosure provides biopolymer detection devices and systems, and methods for forming such devices and systems. A device for detecting a biopolymer comprises a channel that is configured to direct the biopolymer and a pair of electrodes in a portion of the channel. The pair of electrodes has surfaces that are substantially coplanar with adjacent surfaces of the channel. Surfaces of the pair of electrodes are exposed during use of the device to enable detection the biopolymer or a portion thereof with the aid of the pair of electrodes.

### CROSS-REFERENCE

This application is a continuation of International Patent Application No. PCT/JP2015/063403, filed Apr. 28, 2015, which claims priority to Japanese Patent Application Serial No. JP 2014-093079, filed Apr. 28, 2014, and JP 2014-095163, filed May 2, 2014, each of which is entirely incorporated herein by reference.

### DESCRIPTION OF THE RELATED ART

Recently, electrode structures in which a nanoscale gap is provided between opposing electrodes (hereinafter, referred to as a “nanogap electrode device”) have been of interest, and active research is being conducted in electronic devices, bio-devices (biotechnology-devices), etc., in which a nanogap electrode device is used. For example, in the field of biodevices, an analyzer for analyzing the base sequences of deoxyribonucleic acid (DNA) using a nanogap electrode device has been considered (see, for example, WO 2011/108540, which is entirely incorporated herein by reference).

In practice, the analyzer allows a single-stranded DNA molecule to pass through a nanoscale gap (hereinafter, referred to as a “nanogap”) between the electrodes of a nanogap electrode device, the analyzer measures current flowing through the electrodes during passage of each of the bases of the single-stranded DNA molecule as it passes through the nanogap between the electrodes, and the analyzer thereby identifies the bases that constitute the single-stranded DNA molecule based on the current values.

For such an analyzer, the smaller the distance between the electrodes of the nanogap electrode device, the higher the current value that can be detected thereby. This enables analysis of a sample with high sensitivity. However, passing an object to be measured such as a single-stranded DNA molecule through a nanogap between the electrodes is made difficult.

Thus, a nanogap electrode device, having a channel that may cause an object to be measured to pass through a nanogap, is being developed. For example, a nanogap electrode device, in which two electrodes, facing each other across a nanogap, formed on a substrate, and a channel, in communication with the nanogap, also formed on the substrate, has been described in, for example, JP 2009-210272, which is entirely incorporated herein by reference.

### SUMMARY OF THE INVENTION

Methods for producing a nanogap electrode device may comprise forming a pattern in a metal mask made of, for example, titanium, that is formed on an electrode layer made of, for example, gold, the pattern formed by irradiation of a focused ion beam, followed by dry-etching the electrode layer, which is a lower layer exposed by the formation of the pattern in the metal mask so that a nanogap is formed in the electrode layer (see, for example, JP 2004-247203 A, which is entirely incorporated herein by reference).

However, in a nanogap electrode device produced by the method described above, a slot-like nanogap may be formed in an electrode layer by dry-etching the surface of the electrode layer exposed through an opening of the patterned metal mask. Therefore, the minimum width of a gap (gap width in the mask) that can be formed in the electrode layer is the width of a pattern or opening that can be formed in the metal mask. Such method may suffer from a problem in that it can be difficult to form a nanogap that is narrower than a width by which a pattern or opening can be formed in the metal mask. There is a need to develop new nanogap electrode device production methods that are capable of forming nanogap(s) having width(s) that may be substantially narrower than conventionally formed nanogap(s), as well as nanogap(s) having width(s) that may be the same as conventionally formed nanogap(s), as required depending on intended use.

Devices, systems and methods provided herein are capable of producing a nanogap forming component, by which a nanogap can be formed without using a metal mask.

An aspect of the present disclosure provides a device for detecting a biopolymer, comprising: a channel that is configured to direct the biopolymer, wherein a width of the channel is less than 10 nanometers (nm); and a pair of electrodes in a portion of the channel, wherein the pair of electrodes have surfaces that are substantially coplanar with adjacent surfaces of the channel, which surfaces of the pair of electrodes are exposed during use of the device to enable detection the biopolymer or a portion thereof with the aid of the pair of electrodes.

In some embodiments of aspects provided herein, the width is less than 5 nm. In some embodiments of aspects provided herein, the width is less than 2 nm. In some embodiments of aspects provided herein, the width is less than 1 nm. In some embodiments of aspects provided herein, the pair of electrode include tips separated by a gap, which gap has a spacing that is less than the width. In some embodiments of aspects provided herein, the spacing is from 0.5 to 2 times a molecular diameter of the biopolymer. In some embodiments of aspects provided herein, the spacing is from 0.5 to less than a molecular diameter of the biopolymer. In some embodiments of aspects provided herein, the device further comprises a control system in electrical communication with the pair of electrodes, wherein the control system (i) receives signals from the pair of electrodes and (ii) uses the signals to detect the biopolymer or a portion thereof. In some embodiments of aspects provided herein, the channel includes multiple pairs of electrodes with surface that are coplanar with adjacent surfaces of the channel. In some embodiments of aspects provided herein, the pair of electrodes has a gap that is within 2 nm of the width.

Another aspect of the present disclosure provides a device for biopolymer detection, comprising: a first electrode-embedded layer comprising an insulating material, the first electrode-embedded layer having a first electrode-forming face; a second electrode-embedded layer comprising an insulating material, the second electrode-embedded layer having a second electrode-forming face that faces the first electrode-forming face; a first electrode and a second electrode, wherein the first electrode has a first electrode side surface that is exposed within the first electrode-forming face, and wherein the second electrode has a second electrode side surface that is exposed within the second electrode-forming face; and a channel that is at least partially defined by the first electrode-forming face and the second electrode-forming face, wherein the channel (i) extends along a center line between the first electrode-forming face and the second electrode-forming face and (ii) has a width that is substantially constant, wherein the first electrode side surface and the second electrode side surface are disposed in at most a portion of the channel, and wherein the first electrode side surface and second electrode side surface are spaced apart by a gap that has a distance that is substantially the same as the width.

In some embodiments of aspects provided herein, the first electrode-forming face and the first electrode side surface are contiguous. In some embodiments of aspects provided herein, the second electrode-forming face and the second electrode side surface are contiguous. In some embodiments of aspects provided herein, the width is less than 10 nanometers. In some embodiments of aspects provided herein, the gap is substantially within 2 nanometers of the width. In some embodiments of aspects provided herein, the channel is band-like. In some embodiments of aspects provided herein, the channel is substantially straight or curved. In some embodiments of aspects provided herein, the gap is disposed between ends of the channel. In some embodiments of aspects provided herein, the device further comprises a fluid supply member and a fluid discharge member in fluid communication with the channel, wherein each of the fluid supply member and fluid discharge member has a width greater than the width of the channel. In some embodiments of aspects provided herein, the second electrode-embedded layer is on a lower spacer layer.

Another aspect of the present disclosure provides a system for detecting a biopolymer, comprising any of the devices described above or elsewhere herein that detects the biopolymer based on electrical current measured using electrodes of the device. In some embodiments of aspects provided herein, the system further comprises a control system that (i) receives signals from the electrodes and (ii) uses the signals to detect or analyze the biopolymer or a portion thereof.

Another aspect of the present disclosure provides a system for detecting a biopolymer, comprising: at least two devices, wherein each device is as described above or elsewhere herein, and adjacent channels of the at least two devices are in fluid communication with one another.

Another aspect of the present disclosure provides a method for forming a device for detecting a biopolymer, comprising: (a) providing a wall-like sidewall spacer between a first process layer and a second process layer; (b) forming a first electrode-embedded layer from the first process layer by providing the first electrode adjacent to a surface of the first process layer so as to contact a part of the sidewall spacer; (c) forming a second electrode-embedded layer from the second process layer by providing the second electrode adjacent to a surface of the second process layer, wherein the second electrode faces the first electrode across the wall-like sidewall spacer; and (d) removing the wall-like sidewall spacer to provide (i) a nanogap between the first electrode and the second electrode, and (ii) a channel in fluid communication with the nanogap, wherein the nanogap and the channel conform to a shape of the wall-like sidewall spacer.

In some embodiments of aspects provided herein, providing the wall-like sidewall spacer between the first process layer and the second process layer comprises: forming a side surface adjacent to the first process layer; forming a step-like sidewall spacer-forming layer over the side surface; etching back the step-like sidewall spacer-forming layer to form the wall-like sidewall spacer along the side surface of the first process layer; and forming the second process layer to face the first process layer across the wall-like sidewall spacer, thereby providing the wall-like sidewall spacer in a substantially erect manner between the first process layer and the second process layer.

In some embodiments of aspects provided herein, providing the wall-like sidewall spacer between the first process layer and the second process layer comprises: forming a side surface adjacent to the first process layer; forming a step-like sidewall spacer-forming layer over the side surface; forming the second process layer adjacent to the sidewall spacer-forming layer; and conducting a planarization process to expose surfaces of the first process layer and the second process layer, thereby providing the wall-like sidewall spacer in a substantially erect manner between sad first process layer and the second process layer. In some embodiments of aspects provided herein, the method further comprises, prior to (d): forming an electrode-forming mask having openings therein adjacent to the first process layer, adjacent to the sidewall spacer, and adjacent to the second process layer; etching surfaces of the first process layer and the second process layer that are exposed from the openings to form a first electrode embedment recess in the first process layer and a second electrode embedment recess in the second process layer; and forming an electrode layer in portions of the first and second electrode embedment recesses that are exposed through the openings; and removing the electrode-forming mask to form the first electrode in the first electrode embedment recess and the second electrode in the second electrode embedment recess. In some embodiments of aspects provided herein, the removing in (d) comprises forming a solution supply and discharge recess at each end of the wall-like sidewall spacer, and subsequently removing the wall-like sidewall spacer. In some embodiments of aspects provided herein, exposed surfaces of the first and second electrodes are coplanar with surfaces of the channel. In some embodiments of aspects provided herein, the nanogap is disposed in at most a portion of the channel.

Another aspect of the present disclosure provides a method for forming of a nanogap electrode device, comprising: (a) forming a sidewall spacer adjacent to a side face of a step part formed adjacent to a substrate; (b) removing the step part, thereby providing the sidewall spacer in an erect manner adjacent to the substrate; (c) forming a first electrode and a second electrode facing each other across the sidewall spacer; and (d) removing the sidewall spacer, so that a nanogap having a width adjusted by a film thickness of the sidewall spacer is formed between the first electrode and the second electrode.

In some embodiments of aspects provided herein, the method further comprises: forming a mask layer over the sidewall spacer and the substrate that remains exposed; exposing a surface of the step part, a surface of the sidewall spacer, and a surface of the mask layer by a planarization process such that the sidewall spacer is provided in an erect manner adjacent to the substrate between the step part and the mask layer; patterning the step part and the mask layer to provide a patterned step part and a patterned mask layer; and forming the first electrode and the second electrode using the patterned step part and the patterned mask layer as electrode-forming masks. In some embodiments of aspects provided herein, the sidewall spacer formed adjacent to the side face of the step part is formed by etching back a sidewall spacer forming layer. In some embodiments of aspects provided herein, the sidewall spacer extends in a single direction between an electrode tip portion of the first electrode and an electrode tip portion of the second electrode, and a portion of the sidewall spacer is angled along a direction that is different than the single direction. In some embodiments of aspects provided herein, (b) comprises forming a pair of separately-arranged electrode-forming layers adjacent to the substrate, and (c) comprises forming the first electrode and the second electrode facing each other across the sidewall spacer by growing the electrode-forming layers until the electrode-forming layers extend from a surface of the substrate and abut to the sidewall spacer. In some embodiments of aspects provided herein, the first electrode and the second electrode are formed of a material that is different than a metal material of which the electrode-forming layers are formed. In some embodiments of aspects provided herein, upon removing the sidewall space in (d), a channel having a width adjusted by the film thickness of the sidewall spacer is formed, and the first electrode and the second electrode are in at most a portion of the channel. In some embodiments of aspects provided herein, exposed surfaces of the first electrode and the second electrode are coplanar with surfaces of the channel.

Another aspect of the present disclosure provides a method for forming a device for detecting a biomolecule, comprising: (a) integrally forming a lower spacer adjacent to a substrate and a sidewall spacer at an end of the lower spacer, the lower spacer being substantially parallel to a surface of the substrate, the sidewall spacer being substantially perpendicular to the surface of the substrate; (b) forming a first electrode adjacent to the substrate and a second electrode adjacent to the lower spacer such that the second electrode is arranged opposite to the first electrode across the sidewall spacer; (c) partly removing the lower spacer such that the lower spacer remains only between the substrate and the second electrode; and (d) removing the sidewall spacer, thereby forming a nanogap between (i) the first electrode and the second electrode and (ii) the first electrode and the lower spacer, wherein the nanogap has a width adjusted by a film thickness of the sidewall spacer. In some embodiments of aspects provided herein, (a) comprises: forming a sidewall spacer-forming layer over a step part adjacent to the substrate, which sidewall spacer-forming layer overlies the substrate that remains exposed; forming a mask layer over the sidewall spacer-forming layer; using a planarization process to remove a part of the mask layer and the sidewall spacer-forming layer formed over the step part, wherein the sidewall spacer-forming layer remains between the step part and the mask layer such that the sidewall spacer is formed between the step part and the mask layer. In some embodiments of aspects provided herein, the lower spacer remains between the substrate and the mask layer such that the lower spacer is formed between the substrate and the mask layer, and the sidewall spacer is integrally formed with the lower spacer at an end of the lower spacer in an erect manner adjacent to the substrate subsequent to removal of at least a portion of the step part and the mask layer. In some embodiments of aspects provided herein, the sidewall spacer-forming layer remains between the substrate and the mask layer such that the lower spacer is formed between the substrate and the mask layer, and (b) comprises (i) patterning the step part and the mask layer to provide a patterned step part and a patterned mask layer, and (ii) forming the first electrode and the second electrode using the patterned step part and the patterned mask layer as electrode-forming masks. In some embodiments of aspects provided herein, the sidewall spacer and the lower spacer are formed of a conductive material. In some embodiments of aspects provided herein, (a) comprises forming a first electrode-forming layer adjacent to the substrate and a second other electrode-forming layer adjacent to the lower spacer, and (b) comprises forming the first electrode and the second electrode facing each other across the sidewall spacer by growing the electrode-forming layers until the electrode-forming layers abut the sidewall spacer. In some embodiments of aspects provided herein, upon removing the sidewall space in (d), a channel having a width adjusted by the film thickness of the sidewall spacer is formed, and the first electrode and the second electrode are in at most a portion of the channel. In some embodiments of aspects provided herein, exposed surfaces of the first electrode and the second electrode are coplanar with surfaces of the channel. In some embodiments of aspects provided herein, the width is less than 10 nanometers (nm). In some embodiments of aspects provided herein, the width is less than 2 nm. In some embodiments of aspects provided herein, the first electrode and the second electrode are formed of a metal material, and the metal material is replaced by another metal material that is different than the metal material. In some embodiments of aspects provided herein, the sidewall spacer has a width that is less than or equal to 1000 nanometers.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTIONS OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 is a schematic view illustrating a configuration of an example nanogap electrode device produced according to methods for production;

FIGS. 2A-2F are schematic views of steps in an example method for production of a nanogap electrode device;

FIGS. 3A-3F are schematic views of steps in an example method for production of a nanogap electrode device;

FIGS. 4A-4D are schematic views of steps in an example method for production of a nanogap electrode device;

FIGS. 5A-5F are schematic views of steps in an example method for production of a nanogap electrode device;

FIGS. 6A-6D are schematic views of steps in an example method for production of a nanogap electrode device;

FIGS. 7A-7F are schematic views of steps in an example method for production of a modification of a nanogap electrode device;

FIG. 8 is a schematic view illustrating a configuration of an example nanogap electrode device produced by a Production Method;

FIGS. 9A-9F are schematic views of steps in an example method for production of a nanogap electrode device;

FIGS. 10A-10F are schematic views of steps in an example method for production of a nanogap electrode device;

FIGS. 11A-11D are schematic views of steps in an example method for production of a nanogap electrode device;

FIGS. 12A-12F are schematic views of steps in an example method for production of a modification of a nanogap electrode device;

FIGS. 13A-13D are schematic views of steps in an example method for production of a modification of a nanogap electrode device;

FIGS. 14A-14F are schematic views of steps in an example method for production of a nanogap electrode device;

FIGS. 15A-15C are schematic views illustrating configurations of an example sidewall spacer having bent portions;

FIG. 16 is a schematic view of steps in an example method for production of a nanogap electrode device when a substrate having an electrode-forming layer is used;

FIG. 17 is a schematic view illustrating a configuration of an example nanogap electrode device;

FIGS. 18A-18F are schematic views illustrating an example production process for a nanogap electrode device;

FIGS. 19A-19F are schematic views illustrating an example production process for a nanogap electrode device;

FIGS. 20A-20F are schematic views illustrating an example production process for a nanogap electrode device;

FIGS. 21A-21F are schematic views illustrating an example production process for a nanogap electrode device;

FIGS. 22A-22D are schematic views illustrating an example production process for a nanogap electrode device;

FIGS. 23A-23F are schematic views illustrating an example production process for a nanogap electrode device;

FIG. 24 is a schematic view illustrating a configuration of an example nanogap electrode device;

FIGS. 25A-25F are schematic views illustrating an example production process for a nanogap electrode device;

FIGS. 26A-26F are schematic views illustrating an example production process for a nanogap electrode device;

FIGS. 27A-27D are schematic views illustrating an example production process for a nanogap electrode device;

FIG. 28 is a schematic view illustrating a configuration of an example composite nanogap electrode device; and

FIG. 29 shows a computer system that is programmed or otherwise configured to implement methods of the present disclosure.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “gap,” as used herein, generally refers to a pore, channel or passage formed or otherwise provided in a material. The material may be a solid state material, such as a substrate. The gap may be disposed adjacent or in proximity to a sensing circuit or an electrode coupled to a sensing circuit. In some examples, a gap has a characteristic width or diameter on the order of 0.1 nanometers (nm) to about 1000 nm. A gap having a width on the order of nanometers may be referred to as a “nano-gap” (also “nanogap” herein). In some situations, a nano-gap has a width that is from about 0.1 nanometers (nm) to 50 nm, 0.5 nm to 30 nm, or 0.5 nm or 10 nm, 0.5 nm to 5 nm, or 0.5 nm to 2 nm, or no greater than 2 nm, 1 nm, 0.9 nm, 0.8 nm, 0.7 nm, 0.6 nm, or 0.5 nm. In some cases, a nano-gap has a width that is at least about 0.5 nm, 0.6 nm, 0.7 nm, 0.8 nm, 0.9 nm, 1 nm, 2 nm, 3 nm, 4 nm, or 5 nm. The width may be from about 0.5 to 10 times, 0.5 to 5 times, 0.5 to 2 times, or 0.5 to less than a molecular diameter of a biomolecule (e.g., biopolymer), an average molecular diameter of the biomolecule, or a molecular diameter or average molecular diameter of a subunit (e.g., nucleotide) of the biomolecule. In some cases, the width of a nano-gap can be less than a diameter of a biomolecule or a subunit (e.g., monomer) of the biomolecule.

The term “electrode,” as used herein, generally refers to a material or part that can be used to measure electrical current. An electrode (or electrode part) can be used to measure electrical current to or from another electrode. In some situations, electrodes can be disposed in a channel (e.g., nanogap) and be used to measure the current across the channel. The current can be a tunneling current. Such a current can be detected upon the flow of a biomolecule (e.g., protein) through the nano-gap. In some cases, a sensing circuit coupled to electrodes provides an applied voltage across the electrodes to generate a current. As an alternative or in addition to, the electrodes can be used to measure and/or identify the electric conductance associated with a biomolecule (e.g., an amino acid subunit or monomer of a protein). In such a case, the tunneling current can be related to the electric conductance.

The term “biomolecule,” as used herein generally refers to any biological material that can be interrogated with an electrical current and/or potential across a nano-gap electrode. A biomolecule can be a nucleic acid molecule, protein, or carbohydrate. A biomolecule can include one or more subunits, such as nucleotides or amino acids. A biomolecule can be a biopolymer, such as deoxyribonucleic acid (DNA) or ribonucleic acid (RNA).

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is DNA or RNA, or derivatives thereof. A nucleic acid may be single-stranded or double stranded.

The term “protein,” as used herein, generally refers to a biological molecule, or macromolecule, having one or more amino acid monomers, subunits or residues. A protein containing 50 or fewer amino acids, for example, may be referred to as a “peptide.” The amino acid monomers can be selected from any naturally occurring and/or synthesized amino acid monomer, such as, for example, 20, 21, or 22 naturally occurring amino acids. In some cases, 20 amino acids are encoded in the genetic code of a subject. Some proteins may include amino acids selected from about 500 naturally and non-naturally occurring amino acids. In some situations, a protein can include one or more amino acids selected from isoleucine, leucine, lysine, methionine, phenylalanine, threonine, tryptophan and valine, arginine, histidine, alanine, asparagine, aspartic acid, cysteine, glutamine, glutamic acid, glycine, proline, serine and tyrosine.

The term “layer,” as used herein, refers to a layer of atoms or molecules on a substrate. In some cases, a layer includes an epitaxial layer or a plurality of epitaxial layers. A layer may include a film or thin film. In some situations, a layer is a structural component of a device (e.g., light emitting diode) serving a predetermined device function, such as, for example, an active layer that is configured to generate (or emit) light. A layer generally has a thickness from about one monoatomic monolayer (ML) to tens of monolayers, hundreds of monolayers, thousands of monolayers, millions of monolayers, billions of monolayers, trillions of monolayers, or more. In an example, a layer is a multilayer structure having a thickness greater than one monoatomic monolayer. In addition, a layer may include multiple material layers (or sub-layers). In an example, a multiple quantum well active layer includes multiple well and barrier layers. A layer may include a plurality of sub-layers. For example, an active layer may include a barrier sub-layer and a well sub-layer.

The term “adjacent” or “adjacent to,” as used herein, includes ‘next to’, ‘adjoining’, ‘in contact with’, and ‘in proximity to’. In some instances, adjacent to components are separated from one another by one or more intervening layers. For example, the one or more intervening layers can have a thickness less than about 10 micrometers (“microns”), 1 micron, 500 nanometers (“nm”), 100 nm, 50 nm, 10 nm, 1 nm, or less. In an example, a first layer is adjacent to a second layer when the first layer is in direct contact with the second layer. In another example, a first layer is adjacent to a second layer when the first layer is separated from the second layer by a third layer.

The term “substrate,” as used herein, refers to any workpiece on which film or thin film formation is desired. A substrate includes, without limitation, silicon, germanium, silica, sapphire, zinc oxide, carbon (e.g., graphene), SiC, AlN, GaN, spinel, coated silicon, silicon on oxide, silicon carbide on oxide, glass, gallium nitride, indium nitride, titanium dioxide and aluminum nitride, a ceramic material (e.g., alumina, AlN), a metallic material (e.g., molybdenum, tungsten, copper, aluminum), and combinations (or alloys) thereof. A substrate can include a single layer or multiple layers.

The term “contiguous,” as used herein, generally means being in contact, next to, or touching or connected along a boundary or at a point in an unbroken manner.

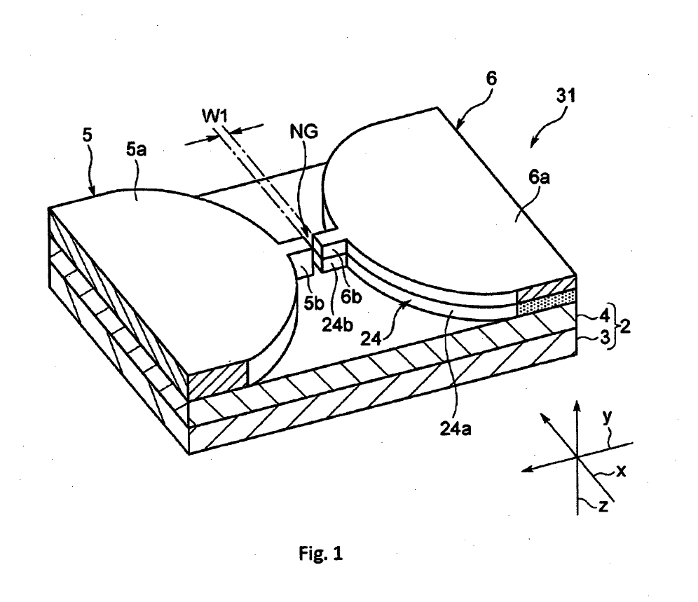

FIG. 1 illustrates an example nanogap electrode device 31, which may have a first electrode(s) 5 and second electrode(s) 6 which may be provided in an opposed manner on a substrate(s) 2, and a nanogap(s) NG of a nanoscale width(s) W1 (for example, of 1000 nm or less, 100 nm or less, 10 nm or less, 1 nm or less, 0.8 nm or less, 0.6 nm or less, or less than the width(s) of a target molecule(s) which may be measured) that may be formed between a first electrode(s) 5 and a second electrode(s) 6. Nanogap electrode device(s) 1 thereby produced may be formed with a nanogap(s) NG having a width(s) W1, for example, from 5 to 30 nm, 2 nm or less, or 1 nm or less, as required according to intended use.

A substrate(s) 2 may be formed of, for example, silicon substrate 3 and silicon oxide layer(s) 4 formed on silicon substrate 3, and it may be configured so that first electrode(s) 5 and second electrode(s) 6 to be paired may be formed on silicon oxide layer(s) 4. First electrode(s) 5 and second electrode(s) 6 may be each made of metal material(s), for example, titanium nitride (TiN), and may be formed on a substrate so as to be substantially left-right symmetrical with a nanogap(s) NG as a center. In some cases, first electrode(s) 5 and second electrode(s) 6 may have a configuration comprising an electrode tip portions 5b, 6b and base portions 5a, 6a that may be integrally formed with an electrode tip portions 5b, 6b at a bottom thereof. Nanogap(s) NG may be defined by electrode tip portions 5b and 6b. Electrode tip portions 5b and 6b may be, for example, each formed in a rectangular parallelepiped shape(s) the longitudinal direction(s) of which may extend a in a y-direction, and may be arranged so that end faces thereof may be opposed.

In an example, tips can be formed by deposition (e.g., vapor deposition or electrochemical deposition). In another example, tips can be formed by induced field emission (e.g., upon application of a voltage across a nanogap.

Base portions 5a, 6a may have a bulge at a central distal end portion at which electrode tip portions 5b, 6b may be provided, and a gentle curved surface may be formed from a central distal end portion toward both sides so that a curved shape with electrode tip portions 5b, 6b as apex(es) may be formed. First electrode(s) 5 and second electrode(s) 6 may be configured so that when a solution, for example, containing at least a single-stranded DNA molecule(s), may be supplied from a y-direction that is a longitudinal direction of first electrode(s) 5 and second electrode(s) 6 or may be supplied from an x-direction that may be perpendicular to a y-direction and may be perpendicular to a z-direction which may be the vertical direction of first electrode(s) 5 and second electrode(s) 6, a solution may be guided toward the electrode tip portions 5b and 6b along a curved surface of base portions 5a and 6a, so that a solution may be reliably passed through (or near) nanogap(s) NG.

Nanogap electrode device(s) 1 having such a configuration may be configured, for example, so that current may be supplied from power supply(ies) (not shown) to first electrode(s) 5 and second electrode(s) 6, and values of current(s) flowing through first electrode(s) 5 and second electrode(s) 6 may be measured by an ammeter(s) (not shown). Nanogap electrode device(s) 1 may allow single-stranded DNA molecule(s) to pass through nanogap(s) NG between first electrode(s) 5 and second electrode(s) 6 from an x direction, and use ammeter(s) to measure the value of current(s) flowing through first electrode(s) 5 and second electrode(s) 6 when each base of single-stranded DNA molecule(s) passes through nanogap(s) NG between first electrode(s) 5 and second electrode(s) 6. Thus, a nanogap electrode device(s) 1 may be capable of identifying bases that constitute single-stranded DNA molecule(s) based on current values.

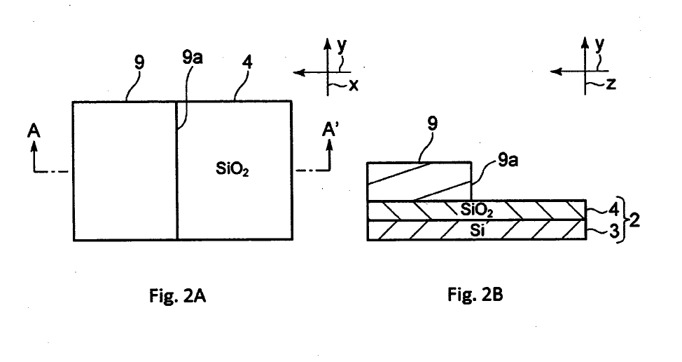

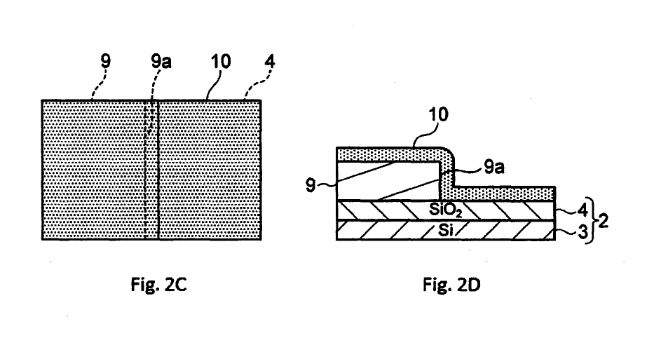

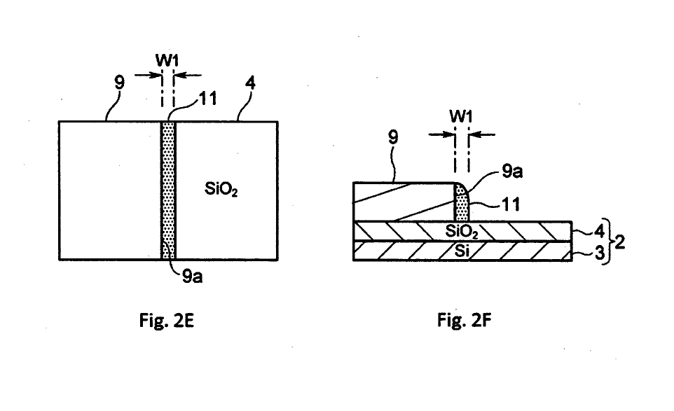

Also provided herein are methods for production of nanogap electrode device(s). First, as shown in FIG. 2A, and as shown in FIG. 2B illustrating a side sectional view taken along the line A-A′ in FIG. 2A, substrate(s) 2, in which silicon oxide layer(s) 4 may be formed as a surface layer on silicon substrate 3, may be provided, and rectangle-shaped step part 9, which may be, for example, formed of silicon nitride (SiN) and which may have a side face 9a, may be formed on silicon oxide layer(s) 4 using a photolithographic technique.

Then, as shown in FIG. 2C, in which similar reference numerals are used to denote parts corresponding to those in FIG. 2A, and as shown in FIG. 2D, in which similar reference numerals are used to denote parts corresponding to those in FIG. 2B, sidewall spacer-forming layer(s) 10 may be deposited on step part(s) 9 and on exposed surface(s) of substrate(s) 2, for example, by a CVD (Chemical Vapor Deposition) method, an ALD (Atomic Layer Deposition) method, a sputtering method, or any other appropriate method. Sidewall spacer-forming layer(s) 10 may be formed using material(s) that may be different from that of a surface of substrate(s) 2 (in this case, silicon oxide layer(s) 4), step part(s) 9, first electrode(s) 5 and second electrode(s) 6 (described later).

For example, when a surface of substrate(s) 2 may be silicon oxide layer(s) 4, step part(s) 9 may be formed of SiN, and first electrode(s) 5 and second electrode(s) 6, described later, may be formed of titanium nitride (TiN), sidewall spacer-forming layer(s) 10 may be formed of titanium (Ti), etc. Furthermore, for example, a surface layer formed on a surface of the substrate(s) 2 may be formed of SiN. In this case, step part(s) 9 may be formed of silicon oxide (SiO2), first electrode(s) 5 and second electrode(s) 6 (described later) may be formed of titanium nitride (TiN), and sidewall spacer-forming layer(s) 10 may be formed of Ti.

At this time, sidewall spacer-forming layer(s) 10 may be formed along a side face 9a of step part(s) 9. A thickness of sidewall spacer-forming layer(s) 10 formed on side face(s) 9a may be determined according to a desired width(s) W1 for nanogap(s) NG. In other words, when forming nanogap(s) NG with a narrow width(s) W1, a film thickness(es) of sidewall spacer-forming layer(s) 10 may be made small, whereas when forming nanogap(s) NG with a large width(s) W1, film thickness(es) of sidewall spacer-forming layer(s) 10 may be made large.

Next, sidewall spacer-forming layer(s) 10, deposited on step part(s) 9 and substrate(s) 2 that remains exposed, may be etched back with a directional etch process, for example, by dry etching so that sidewall spacer-forming layer(s) 10 remains along side face(s) 9a of step part(s) 9. Thus, as shown in FIG. 2E, in which similar reference numerals are used to denote parts corresponding to those in FIG. 2D, and as shown in FIG. 2F, in which similar reference numerals are used to denote parts corresponding to those in FIG. 2D, a sidewall-like independent sidewall spacer(s) 11 may be formed along side face(s) 9a of step part(s) 9. Wall-like sidewall spacer(s) 11 thereby formed may have a shape increasing in width from a top of side face(s) 9a of step part(s) 9 to substrate(s) 2. Maximum thickness(es), i.e., width(s), of sidewall spacer(s) 11 may be a width(s) W1 of nanogap(s) NG that may be formed utilizing sidewall spacer(s) 11. Thus, according to a sidewall spacer production method described herein above, a sidewall spacer(s) 11 may be produced which may be provided in an erect manner on substrate(s) 2 like a wall and which may have thickness(es) of 1,000 nm or less (nanoscale), or 30 nm or less, and furthermore, thickness(es) of 2 nm or less, or 1 nm or less, as required according to intended use.

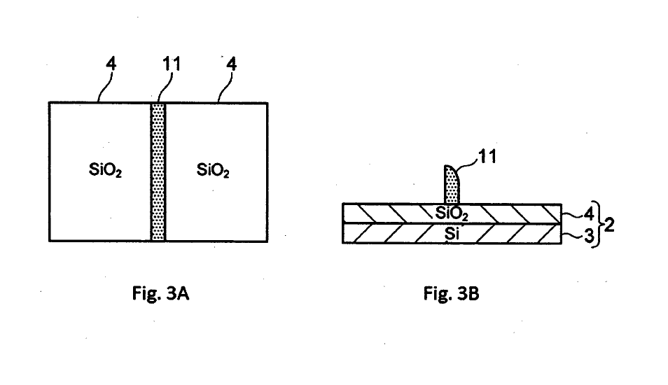

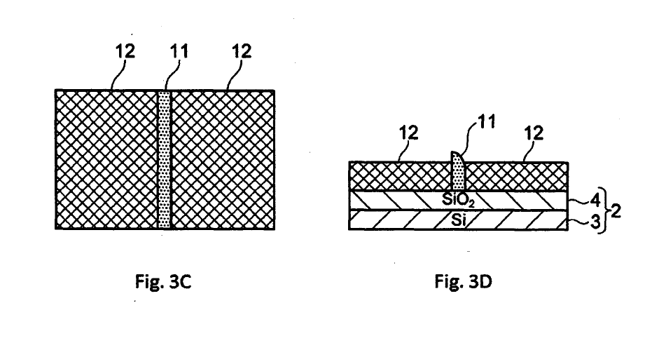

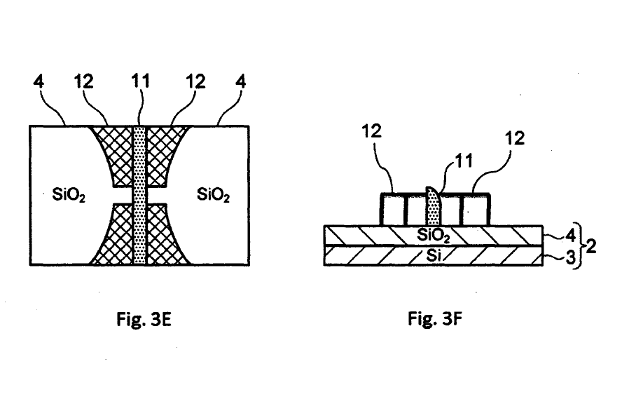

Next, as shown in FIG. 3A, in which similar reference numerals are used to denote parts corresponding to those in FIG. 2E, and as shown in FIG. 3B, in which similar reference numerals are used to denote parts corresponding to those in FIG. 2F, step part(s) 9 may be removed by etching so that the sidewall spacer(s) 11 may be provided in a manner standing up vertically with respect to a surface of substrate(s) 2 at a predetermined position on substrate(s) 2.

In some cases, processing steps described hereinabove and shown in FIGS. 2A to 3B may be utilized for the method for production of sidewall spacer(s) 11. Sidewall spacer(s) 11 may thus be produced which may be used for formation of nanogap(s) NG (described later). Processing steps for forming nanogap(s) NG using such a sidewall spacer(s) 11 formed in a manner standing on substrate(s) 2, and then the nanogap electrode device 1 is produced, are described below.

As shown in FIG. 3C, in which similar reference numerals are used to denote parts corresponding to those in FIG. 3A, and as shown in FIG. 3D, in which similar reference numerals are used to denote parts corresponding to those in FIG. 3B, a resist coating agent may be applied onto silicon oxide layer(s) 4 and may be cured to form a resist layer(s) 12.

Next, as shown in FIG. 3E, in which similar reference numerals are used to denote parts corresponding to those in FIG. 3C, and as shown in FIG. 3F, in which similar reference numerals are used to denote parts corresponding to those in FIG. 3D, certain parts of resist layer(s) 12, corresponding to regions at which first electrode(s) 5 and second electrode(s) 6 may be formed, may be removed using a photolithographic technique, so that a patterned resist layer(s) 12 (an electrode-forming mask) may be formed, whereby silicon oxide layer 4 may be exposed at regions on which first electrode(s) 5 and second electrode(s) 6 may be formed.

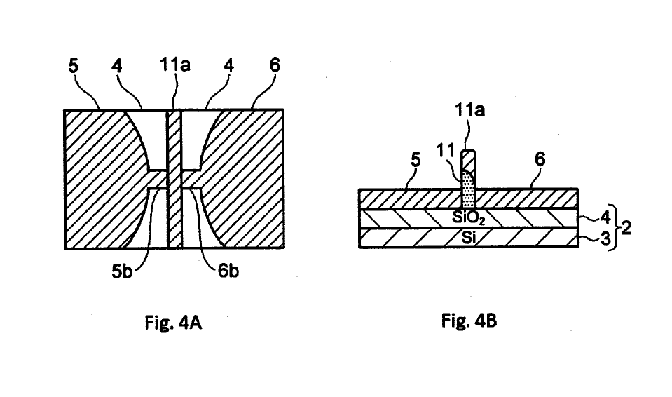

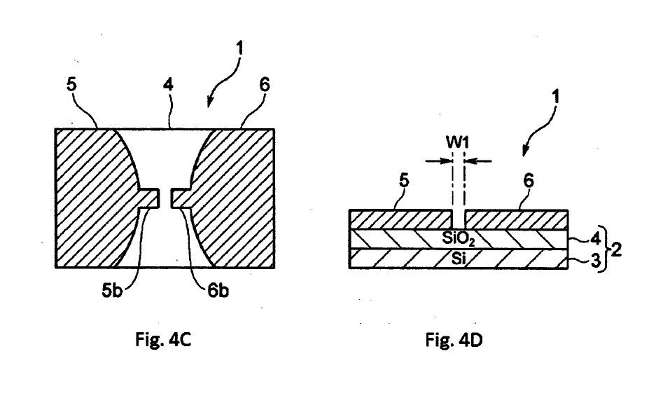

Next, as shown in FIG. 4A, in which similar reference numerals are used to denote parts corresponding to those in FIG. 3E, and as shown in FIG. 4B, in which similar reference numerals are used to denote parts corresponding to those in FIG. 3F, after depositing a metal layer, from which first electrode(s) 5 and second electrode(s) 6 may be formed, on patterned resist layer(s) 12 (the electrode-forming mask) and exposed substrate(s) 2 (silicon oxide layer 4), portions of a metal layer, other than portions corresponding to first electrode(s) 5 and to the second electrode(s) 6 may be removed by a lift-off process, so that first electrode(s) 5 and second electrode(s) 6 may be formed on substrate(s) 2 with electrode tip portions 5b and 6b being arranged facing each other across sidewall spacer(s) 11.

At this time, metal layer(s) 11a may remain on sidewall spacer(s) 11. Remaining metal layer(s) 11a on sidewall spacer(s) 11 may be removed by polishing using a CMP (Chemical Mechanical Polishing) technique, etc. Alternatively, without the need for removal by the CMP technique, etc., at this time, remaining metal layer(s) 11a may be removed together with sidewall spacer(s) 11 when sidewall spacer(s) 11 may be removed later.

Finally, as shown in FIG. 4C, in which similar reference numerals are used to denote parts corresponding to those in FIG. 4A, and as shown in FIG. 4D, in which similar reference numerals are used to denote parts corresponding to those in FIG. 4B, nanogap(s) NG which may have a same width(s) W1 as that of sidewall spacer(s) 11, may be formed between electrode tip portions 5b and 6b by removing sidewall spacer(s) 11, for example, by wet etching. Thus, nanogap electrode device(s) 1 as shown in FIG. 1 may be produced. Sidewall spacer(s) 11 may be formed of a material that is different from a material of a surface of substrate(s) 2, i.e., and or a material of silicon oxide layer 4, and may be different from a material of the first electrode(s) 5 and second electrode(s) 6. Accordingly, it is ensured that only sidewall spacer(s) 11 may be removed, leaving silicon oxide layer 4, and that first electrode(s) 5 and second electrode(s) 6 may remain on substrate(s) 2.

As described herein above, after sidewall spacer(s) 11 may be formed on step part(s) 9 formed on substrate(s) 2, step part(s) 9 may be removed so that sidewall spacer(s) 11 may be provided in an erect manner on substrate(s) 2. After forming a patterned resist layer 12 as a mask on substrate(s) 2, a metal layer(s) may be formed on resist layer(s) 12 and on substrate(s) 2 exposed through the openings in resist layer(s) 12, a metal layer(s) on resist layer(s) 12 may then be removed by removing patterned resist layer(s) 12, so that first electrode(s) 5 and second electrode(s) 6 may be formed on substrate(s) 2 so as to be arranged facing each other across sidewall spacer(s) 11. Finally, in some cases, sidewall spacer(s) 11 may be removed, so that nanogap(s) NG having a same width(s) W1 as that of sidewall spacer(s) 11 may be formed between first electrode(s) 5 and second electrode(s) 6.

As described herein above, nanogap(s) NG having a desired width(s) W1 may be formed by adjusting a film thickness(es) of sidewall spacer(s) 11, and a film thickness(es) of sidewall spacer(s) 11 may be formed very thin. Therefore, nanogap(s) NG having a very small width(s) W1 corresponding to a width(s) W1 of sidewall spacer(s) 11 may also be formed.

In some cases, after providing sidewall spacer(s) 11 in an erect manner on substrate(s) 2, patterned resist layer(s) 12 may be used to form first electrode(s) 5 and second electrode(s) 6 facing each other across sidewall spacer(s) 11, and subsequently, resist layer(s) 12 and sidewall spacer(s) 11 may be removed so that nanogap(s) NG having a width(s), which may be adjusted by a film thickness(es) of sidewall spacer(s) 11, may be formed between first electrode(s) 5 and second electrode(s) 6. Thus, by adjusting film thickness(es) of sidewall spacer(s) 11, nanogap(s) NG having a same width(s) W1 as that of a conventionally formed nanogap(s) may be formed, and furthermore, nanogap(s) NG having a width(s) W1 that may be substantially narrower than that of a conventionally formed nanogap may also be formed.

In some cases for sidewall spacer production, after forming sidewall spacer(s) 11 on side face(s) 9a of step part(s) 9 formed at predetermined region(s) on substrate(s) 2, step part(s) 9 may be removed so that sidewall spacer(s) 11 may be provided in an erect manner on substrate(s) 2. As a result, after forming first electrode(s) 5 and second electrode(s) 6 facing each other across sidewall spacer(s) 11 that may be provided in an erect manner on substrate(s) 2, sidewall spacer(s) 11 may be removed so that nanogap(s) NG having a same width(s) as that of sidewall spacer(s) 11 may be formed between first electrode(s) 5 and second electrode(s) 6. Thus in some cases of methods for production of sidewall spacer(s) 11, unlike in a conventional formation technique for forming a slot-like nanogap in the surface of an electrode layer by etching the electrode layer exposed from an opening in a metal mask, sidewall spacer(s) 11, by which nanogap(s) NG may be formed without using a conventional metal mask, may be produced.

In some cases, a slot-like gap may be formed in silicon oxide layer 4 below nanogap(s) NG by removing a part of a surface of substrate(s) 2, i.e., a surface of a silicon oxide layer, by use of first electrode(s) 5 and second electrode(s) 6 as masks, after which, nanogap electrode device(s) 1 as shown in FIG. 4C and FIG. 4D may be formed. For nanogap electrode device(s) as described above, electric field(s) may be generated in gap(s) in silicon oxide layer 4 below nanogap(s) NG. When single-stranded DNA molecule(s), which may be a single single-stranded DNA molecule(s), passes through a gap in silicon oxide layer 4, the local conductance may change. In response thereto, values of current(s) flowing through first electrode(s) 5 and second electrode(s) 6 may change. Based on such change(s) in current value(s), bases that constitute a single-stranded DNA molecule may be identified.

In some cases of a production method, step part(s) 9 may be first formed on substrate(s) 2, and then sidewall-like sidewall spacer(s) 11 may be formed along a side face 9a of step part(s) 9 as formed described herein above and as shown in FIG. 2E and FIG. 2F. Processing steps therefor may correspond to those described in association with FIG. 2A to FIG. 2F.

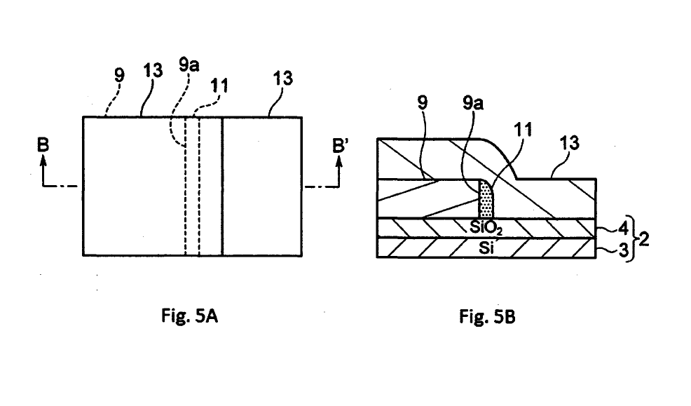

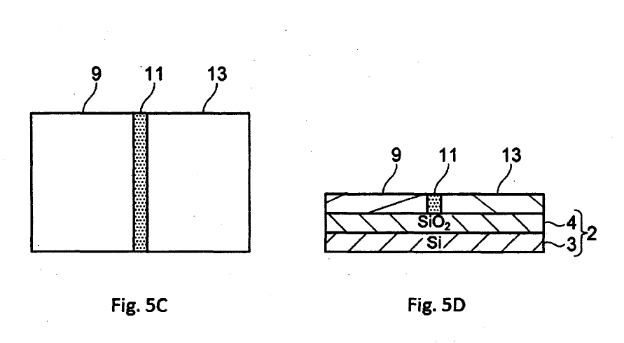

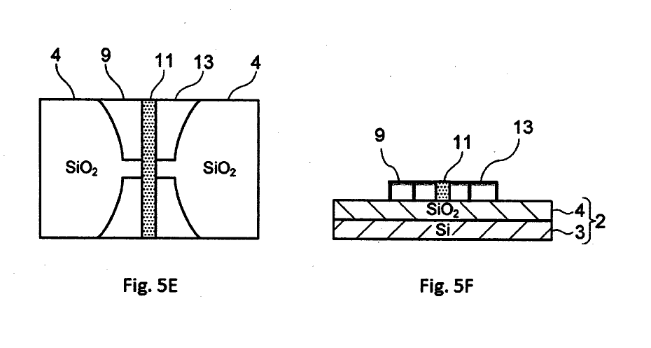

Next, as shown in FIG. 5A, in which similar reference numerals are used to denote parts corresponding to those in FIG. 2E, and as shown in FIG. 5B illustrating a side sectional view taken along the line B-B′ in FIG. 5A, insulating layer(s) 13 (mask layer), which overlies step part(s) 9, sidewall spacer(s) 11, and portions of exposed substrate(s) 2 that remain exposed, may be formed. In some cases, insulating layer(s) 13, which may be formed of an insulating material such as, for example, silicon nitride (SiN), which may be of the same material of step part(s) 9, may be used as a mask layer. However, insulating layer 13 is not limited thereto, and mask layer and step part(s) 9 may also be formed of any material other than the material of insulating layer 13.

Next, as shown in FIG. 5C, in which similar reference numerals are used to denote parts corresponding to those in FIG. 5A, and as shown in FIG. 5D, in which similar reference numerals are used to denote parts corresponding to those in FIG. 5B, surfaces of step part(s) 9, sidewall spacer(s) 11, and insulating layer(s) 13 may all be exposed by over-polishing by a planarizing process such as CMP, etc. As a result, sidewall spacer(s) 11 provided in an erect manner on substrate(s) 2 may be formed between step part(s) 9 and insulating layer(s) 13.

In using a planarizing process, an upper steeply-angled portion of sidewall spacer(s) 11 as viewed from the side may be polished away while overpolishing step part(s) 9, sidewall spacer(s) 11, and insulating layer(s) 13, until a cross sectional shape of sidewall spacer(s) 11 between a step part(s) 9 and insulating layer(s) 13 may be formed so as to have a substantially rectangular cross sectional shape. When a planarizing process is performed, if sidewall spacer(s) 11 with a surface thereof being exposed can be formed between step part(s) 9 and insulating layer(s) 13, only a part of insulating layer(s) 13 overlaying step part(s) 9 and sidewall spacer(s) 11 may be polished away.

Processing steps described above and shown in FIGS. 2A to 2F and FIGS. 5A to 5D, may be used for a method of production of sidewall spacer(s) 11. Sidewall spacer(s) 11 may thus be produced, and may be used for forming a nanogap(s) NG (described elsewhere herein). Then, additional processing steps used for formation of a nanogap(s) NG using such a sidewall spacer(s) 11 provided in an erect manner on the substrate(s) 2, and formation of nanogap electrode device(s) 1 are described below.

After forming a layer-like resist mask (not shown) on exposed surfaces of step part(s) 9, sidewall spacer(s) 11, and insulating layer(s) 13, a photolithographic technique may be used to remove a part of step part(s) 9 and a part of insulating layer(s) 13 so that patterned step part(s) 9 and patterned insulating layer(s) 13 (electrode-forming masks) may be formed, as shown in FIG. 5E, in which similar reference numerals are used to denote parts corresponding to those in FIG. 5C, and as shown in FIG. 5F, in which similar reference numerals are used to denote parts corresponding to those in FIG. 5D. As shown in FIG. 5E and FIG. 5F, a pattern of a part to be removed from step part(s) 9 and a pattern of a part to be removed from insulating layer(s) 13 may correspond to a pattern of first electrode(s) 5 and a pattern of second electrode(s) 6, respectively. Thus, regions of step part(s) 9 and insulating layer(s) 13, at which first electrode(s) 5 and second electrode(s) 6 may be formed, are removed, so that surfaces of the substrate(s) 2 (silicon oxide layer 4) may be exposed.

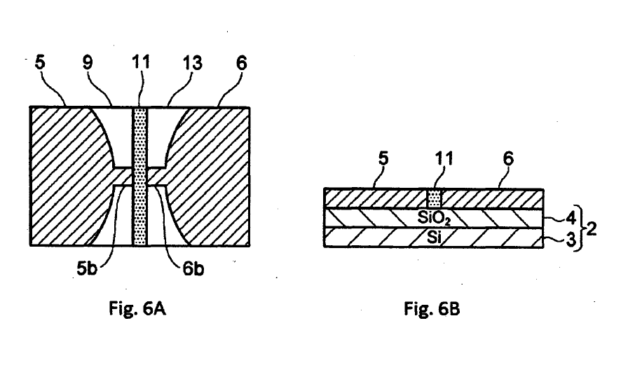

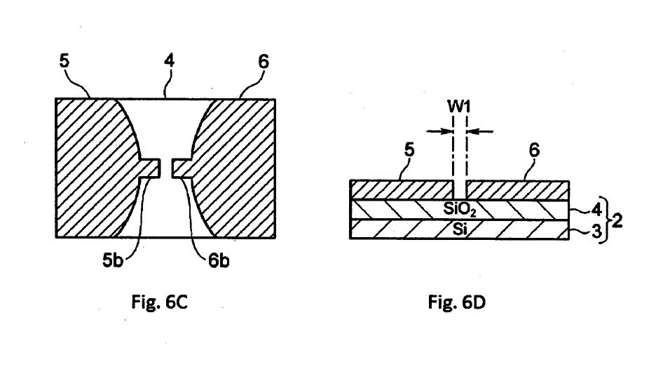

Next, a metal layer may be formed on silicon oxide layer 4 which may be exposed at regions at which first electrode(s) 5 and second electrode(s) 6 are to be formed, and on step part(s) 9 and insulating layer(s) 13 at regions remaining as electrode-forming masks, i.e., regions other than regions at which first electrode(s) 5 and second electrode(s) 6 may be formed. Subsequently, a planarizing process such as CMP, etc., may be performed to expose surfaces of the remaining portions of step part(s) 9, and remaining portions of insulating layer(s) 13, and sidewall spacer(s) 11. As a result, as shown in in FIG. 6A, in which similar reference numerals are used to denote parts corresponding to those in FIG. 5E, and as shown in FIG. 6B, in which similar reference numerals are used to denote parts corresponding to those in FIG. 5F, metal layer(s) on regions on patterned step part(s) 9 and patterned insulating layer(s) 13 (electrode-forming mask) and metal layer(s) on sidewall spacer(s) 11 may be removed, so that first electrode(s) 5 and second electrode(s) 6 may be formed on substrate(s) 2 with electrode tip portions 5b and 6b facing each other across sidewall spacer(s) 11.

Finally, as shown in FIG. 6C, in which similar reference numerals are used to denote parts corresponding to those in FIG. 6A, and as shown in FIG. 6D, in which similar reference numerals are used to denote parts corresponding to those in FIG. 6B, nanogap(s) NG having a same width(s) as that of sidewall spacer(s) 11, may be formed between electrode tip portions 5b and 6b by removing sidewall spacer(s) 11, patterned step part(s) 9, and patterned insulating layer(s) 13, for example, by wet etching. Thus, nanogap electrode device(s) 1 as shown in FIG. 1 may be produced.

As described herein above, after sidewall spacer(s) 11 formed on a side face of step part(s) 9 may be formed at a predetermined region(s) on substrate(s) 2, insulating layer(s) 13, which may overlay step part(s) 9, and may overlay sidewall spacer(s) 11, and may overlay exposed substrate(s) 2, may be formed. Furthermore, in some cases of production methods, a planarizing process may be used to expose a surface of step part(s) 9, a surface of sidewall spacer(s) 11, and a surface of insulating layer(s) 13, so that sidewall spacer(s) 11 may be provided in an erect manner on substrate(s) 2 between step part(s) 9 and insulating layer(s) 13. Then, step part(s) 9 and insulating layer(s) 13 may be patterned. Using so-patterned step part(s) 9 and insulating layer(s) 13 as electrode-forming masks, first electrode(s) 5 and second electrode(s) 6 facing each other across sidewall spacer(s) 11 may be formed. Finally, sidewall spacer(s) 11, patterned step part(s) 9 and patterned insulating layer(s) 13 may be removed, so that nanogap(s) NG having a same width(s) W1 as that of sidewall spacer(s) 11 may be formed between first electrode(s) 5 and second electrode(s) 6.

As described herein above, in some cases of methods for production of a nanogap electrode, nanogap(s) NG having a desired width(s) W1 may be formed by adjusting a film thickness of sidewall spacer(s) 11, and a film thickness(es) of sidewall spacer(s) 11 may be formed so as to be very thin. Therefore, nanogap(s) NG having a small width(s) W1 corresponding to a width(s) W1 of sidewall spacer(s) 11 may also be formed.

In view of the above, in some cases of production methods, after providing sidewall spacer(s) 11 in an erect manner on substrate(s) 2, patterned step part(s) 9 and patterned insulating layer(s) 13 may be used to form first electrode(s) 5 and second electrode(s) 6 facing each other across sidewall spacer(s) 11, and subsequently sidewall spacer(s) 11, patterned step part(s) 9, and patterned insulating layer(s) 13 may be removed, so that nanogap(s) NG having width(s) W1, which may be adjusted by a film thickness(es) of sidewall spacer(s) 11, may be formed between first electrode(s) 5 and second electrode(s) 6. Thus, by adjusting a film thickness of sidewall spacer(s) 11, nanogap(s) NG having a same width(s) W1 conventionally formed nanogap(s) may be formed, and furthermore, nanogap(s) NG having width(s) W1 that may be substantially narrower than conventionally formed nanogap(s) may be formed.

Furthermore, in some cases of sidewall spacer production methods, after sidewall spacer(s) 11 may be produced on a side face of step part(s) 9 formed at predetermined region(s) on substrate(s) 2, insulating layer(s) 13 (mask layer), which overlay(s) step part(s) 9, sidewall spacer(s) 11, and exposed substrate(s) 2, may be formed. Then, a surface of step part(s) 9, a surface of sidewall spacer(s) 11, and a surface of insulating layer(s) 13 may be exposed by a planarizing process, so that sidewall spacer(s) 11 may be provided in an erect manner on substrate(s) 2 between step part(s) 9 and insulating layer(s) 13.

Subsequently, in some cases, first electrode(s) 5 and second electrode(s) 6 may be formed on opposite sides of sidewall spacer(s) 11 that may be provided in an erect manner on substrate(s) 2, and subsequently sidewall spacer(s) 11 may be removed, so that nanogap(s) NG having a same width(s) as that of sidewall spacer(s) 11 may be formed between first electrode(s) 5 and second electrode(s) 6. Thus, in some cases for methods of production of sidewall spacer(s) 11, unlike in conventional formation techniques for forming a slot-like nanogap in the surface of an electrode layer by etching the electrode layer exposed from an opening in a metal mask, sidewall spacer(s) 11, by which a nanogap(s) can be formed without using a conventional metal mask, may also be produced.

In addition to the cases described above, a slot-like gap may be formed in silicon oxide layer(s) 4 below nanogap(s) NG by removing a part of a surface of silicon oxide layer(s) 4 that is an upper layer of substrate(s) 2 by use of first electrode(s) 5 and second electrode(s) 6 as masks, after formation of nanogap electrode device(s) 1 as shown in FIG. 6C and FIG. 6D. For nanogap electrode device(s) as described above, an electric field may be generated in a gap in silicon oxide layer(s) 4 below nanogap(s) NG. When a single-stranded DNA molecule(s) passes through a gap in the silicon oxide layer(s) 4 (one single-stranded DNA molecule at a time), the local conductivity may change. In response thereto, values of current(s) flowing through first electrode(s) 5 and second electrode(s) 6 may change. Based on change(s) in current value(s), bases that constitute single-stranded DNA molecule(s) may be identified.

As another case, before removing sidewall spacer(s) 11 shown in FIG. 4B, a metal material, which may be different from that of first electrode(s) 5 and second electrode(s) 6, may be formed on first electrode(s) 5 and second electrode(s) 6, so that first electrode(s) 5 and second electrode(s) 6 may have be utilized as electrodes having tip region, which may be formed of metal(s) different from that of lower layer(s), as an upper layer.

As another case, before removing sidewall spacer(s) 11 shown in FIG. 4B, first electrode(s) 5 and second electrode(s) 6, which may be first formed of one or more predetermined metal material(s), for example, Ni, etc., may be subjected to gold plating so that a material(s) of first electrode(s) 5 and second electrode(s) 6 which face the nanogap and form the electrode tips may be effectively replaced with metal material(s) such as gold, etc., that may be different from Ni, as a result of said plating.

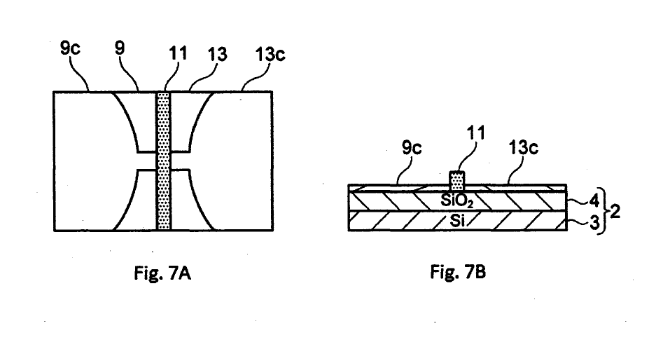

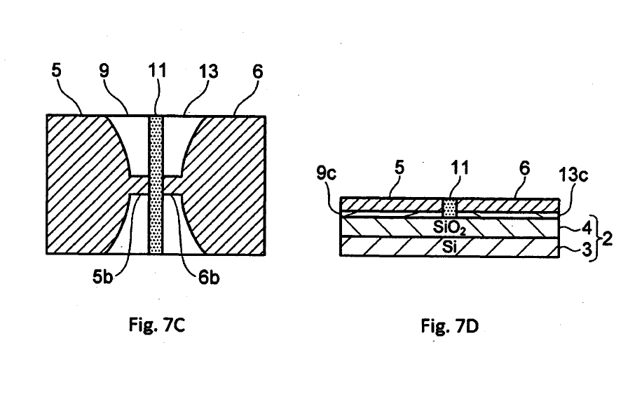

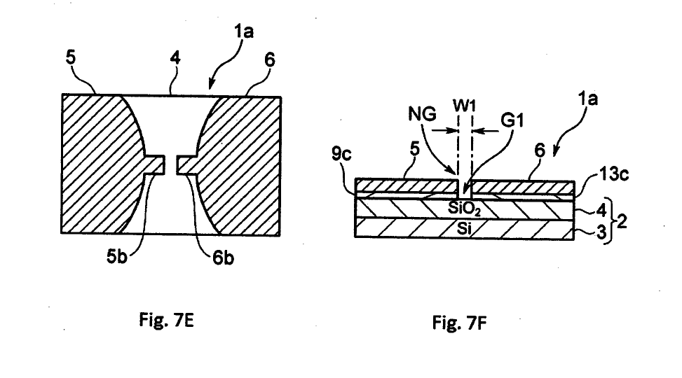

For some cases as described hereinabove, there is described an example in which when step part(s) 9 and insulating layer(s) 13 shown in FIG. 5C and FIG. 5D may be patterned, step part(s) 9 and insulating layer(s) 13 at regions at which first electrode(s) 5 and second electrode(s) 6 may be formed may be removed so that a surface (silicon oxide layer(s) 4) of substrate(s) 2 may be exposed. Other cases are not limited thereto. As shown in FIG. 7A, in which similar reference numerals are used to denote parts corresponding to those in FIG. 5C, and as shown in FIG. 7B, in which similar reference numerals are used to denote parts corresponding to those in FIG. 5D, thin step part(s) 9c obtained by thinning the step part(s) 9 and thin insulating layer(s) 13c obtained by thinning the insulating layer 13 (thin mask layer) may be formed.

Unlike a nanogap electrode device 1 as shown in FIG. 1, nanogap electrodes produced in this manner may have a configuration in which thin step part(s) 9c may be formed between substrate(s) 2 and first electrode(s) 5, and thin insulating layer(s) 13c may be formed between substrate(s) 2 and second electrode(s) 6.

In some cases, after layer-like resist mask(s) (not shown) may be formed on an exposed surface of step part(s) 9, a surface of sidewall spacer(s) 11 and a surface of insulating layer(s) 13 as shown in FIGS. 5C and 5D, a part of a surface of step part(s) 9 and a part of a surface of insulating layer(s) 13, at which first electrode(s) 5 and second electrode(s) 6 (described layer) may be formed respectively, may be removed using a photolithographic technique. Then, as shown in FIGS. 7A and 7B, a thickness(es) of regions at which first and second electrodes 5 and 6 may be formed may be reduced, so that thin step part(s) 9c and thin insulating layer(s) 13c may be formed.

Next, after a metal layer(s) may be formed on thin step part(s) 9c, on thin insulating layer(s) 13c, on remaining step part(s) 9 and insulating layer(s) 13, and on sidewall spacer(s) 11, a planarization process such as CMP, etc., may be performed so that a surface of step part(s) 9 other than at region(s) at which first electrode(s) 5 may be formed, a surface of insulating layer(s) 13 other than at region(s) at which second electrode 6 may be formed, and a surface of sidewall spacer(s) 11 may all be exposed. As a result, as shown in FIG. 7C, in which similar reference numerals are used to denote parts corresponding to those in FIG. 7A, and as shown in FIG. 7D, in which similar reference numerals are used to denote parts corresponding to those in FIG. 7B, metal layer(s) other than regions at which first electrode(s) 5 and second electrode(s) 6 may be formed, and metal layer(s) at a region on sidewall spacer(s) 11 may be removed, so that a metal layer(s) remains on thin step part(s) 9c and on thin insulating layer(s) 13c. Thus, first electrode(s) 5 and second electrode(s) 6 with electrode tip portions 5b and 6b thereof facing each other across sidewall spacer(s) may be formed on substrate(s) 2.

Finally, sidewall spacer(s) 11, step part(s) 9 other than at region(s) at which first electrode(s) 5 may be formed, and insulating layer(s) 13 other than at region at which second electrode(s) 6 may be formed, may be removed, for example, by dry etching. As a result, nanogap(s) NG having a same width(s) W1 as that of sidewall spacer(s) 11 may be formed between first electrode(s) 5 and second electrode(s) 6, and gap(s) G1 sandwiched between thin step part(s) 9c and thin insulating layer(s) 13c may be formed below nanogap(s) NG, as shown in FIG. 7E, in which similar reference numerals are used to denote parts corresponding to those in FIG. 7C, and as shown in FIG. 7F, in which similar reference numerals are used to denote parts corresponding to those in FIG. 7D.

For nanogap electrode device(s) la produced in this way, single-stranded DNA molecule(s) may pass through nanogap(s) NG between first electrode(s) 5 and second electrode(s) 6 (and single-stranded DNA molecule(s) may pass though one at a time), and single-stranded DNA molecule(s) may pass through gap(s) G1 which may be located below nanogap(s) NG and which may sandwiched between thin step part(s) 9c and thin insulating layer(s) 13c. For nanogap electrode device(s) la as described above, electric field(s) may be generated in gap(s) G1 between thin step part(s) 9c formed of an insulating material and thin insulating layer(s) 13c. When single-stranded DNA molecule(s) passes through gap(s) G1 between thin step part(s) 9c formed of an insulating material and thin insulating layer(s) 13c, the local conductivity field(s) may change. In response thereto, values of current(s) flowing through first electrode(s) 5 and second electrode(s) 6 may change. Based on such changes in current values, bases that constitute single-stranded DNA molecule(s) may be identified.

In some cases, slot-like gap(s) may be additionally formed in silicon oxide layer(s) 4 below nanogap(s) NG and gap(s) G1 by removing a part of a surface of silicon oxide layer(s) 4 that may be an upper layer of substrate(s) 2 by use of first electrode(s) 5 and second electrode(s) 6 as masks, after nanogap electrode device(s) la, as shown in FIG. 7E and FIG. 7F, may be produced.

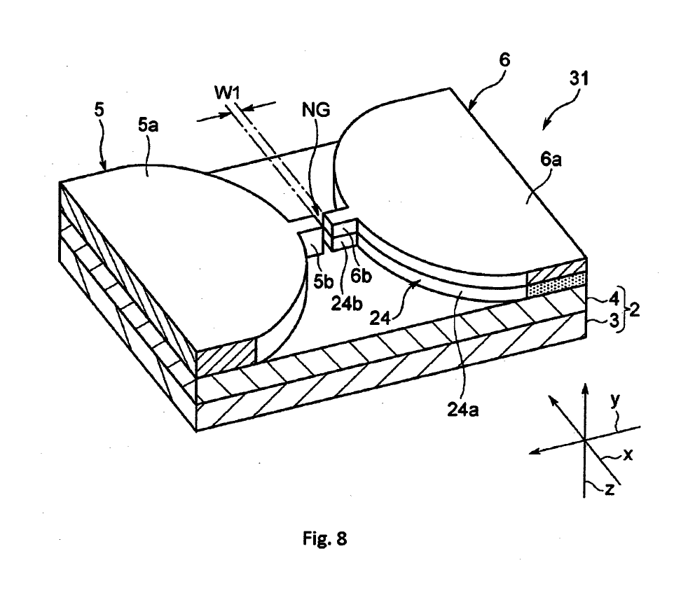

In some cases for production methods of nanogap electrode device as shown in FIG. 8, in which similar reference numerals are used to denote parts corresponding to those in FIG. 1, illustrates a nanogap electrode device(s) 31. The configuration of nanogap electrode device(s) 31 may be different from that of nanogap electrode device(s) 1 as illustrated in FIG. 1 above in that lower spacer(s) 24 may be formed below second electrode(s) 6. Herein, a description will be made below focusing on configuration of second electrode(s) 6 and lower spacer(s) 24.

In some cases, lower spacer(s) 24 may be formed on substrate(s) 2, and may be designed so that second electrode(s) 6 may be stacked thereon. Thus, lower spacer(s) 24 together with second electrode(s) 6 may be arranged opposite to first electrode(s) 5. In some cases, lower spacer 24 may have a same contour shape as a contour shape of second electrode(s) 6. Lower spacer(s) 24 may be constituted of an electrode tip portion(s) 24a and a base portion(s) that may be integrally formed with electrode tip portion(s) 24b at a bottom thereof. Electrode tip portion(s) 24b may, for example, be formed in a rectangular parallelepiped shape with a longitudinal direction thereof extending in y-direction, and may be arranged so that an end face thereof may be opposed to an end face of an electrode tip portion of first electrode(s) 5.

First electrode(s) 5, second electrode(s) 6, and lower spacer(s) 24 may be configured so that when a solution, for example, containing single-stranded DNA molecule(s), may be supplied from an aforementioned y-direction or may be supplied from an x-direction that may be perpendicular to a y-direction and perpendicular to a z-direction which may be a height direction, a solution may be guided toward electrode tip portions 5b, 6b, and 24b, along curved surfaces of base portions 6a and 24a, so that a solution may be passed through nanogap(s) NG between electrode tip portion(s) 5b and electrode tip portions(s) 6b, 24b.

Lower spacer(s) 24 may be formed of a conductive material. Lower spacer(s) 24, as well as second electrode(s) 6, may be supplied with current(s) from power source(s) (not shown). This allows nanogap electrode device(s) 31 to pass single-stranded DNA molecule(s) from a x-direction through nanogap(s) NG between first electrode(s) 5 and second electrode(s) and also between first electrode(s) 5 and lower spacer(s) 24, while first electrode(s) 5 and a pair(s) of second electrode(s) 6 and lower spacer(s) 24 may be supplied with current(s) from power supply(ies). When bases of single-stranded DNA molecule(s) pass through nanogap(s) NG, values of current(s) flowing through first electrode(s) 5 and second electrode(s) 6 and also through between first electrode(s) 5 and lower spacer(s) 24, may be measured by an ammeter(s). Thus, bases that constitute single-stranded DNA molecule(s) may be identified based on current values.

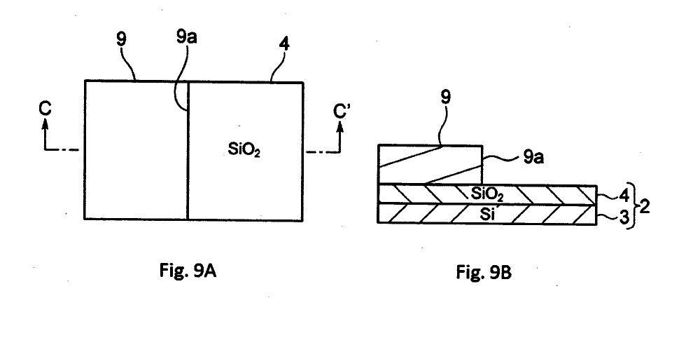

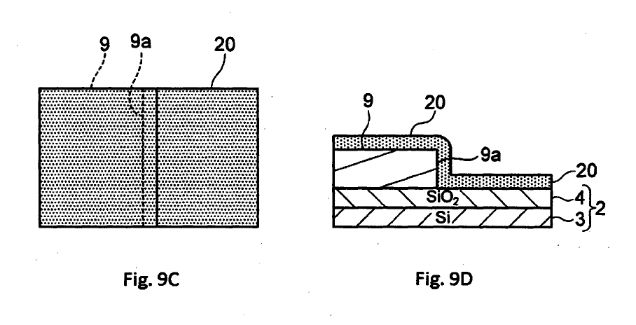

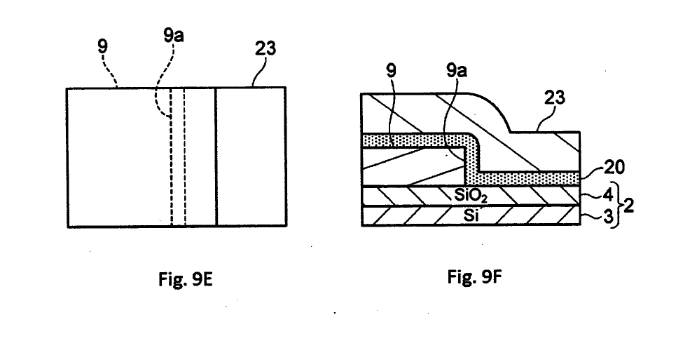

Next, a method for production of nanogap electrode device(s) 31 shown in FIG. 8 is described below. First, as shown in FIG. 9A, in which similar reference numerals are used to denote parts corresponding to those in FIG. 2A, and as shown in FIG. 9B, illustrating a side sectional view taken along the line C-C′ in FIG. 9A, substrate(s) 2 in which silicon oxide layer(s) 4 may be formed on a silicon substrate(s) 3 may be provided, and rectangular-shaped step part(s) 9, which may, for example, be formed of silicon nitride (SiN) and which may have a side face(s) 9a, may be formed on silicon oxide layer(s) 4 using a photolithographic technique.

Then, as shown in FIG. 9C, in which similar reference numerals are used to denote parts corresponding to those in FIG. 9A, and as shown in FIG. 9D, in which similar reference numerals are used to denote parts corresponding to those in FIG. 9B, sidewall spacer-forming layer(s) 20 may be formed of a material such as titanium nitride (TiN) that may be different from a material of a surface (in this case, a silicon oxide layer(s) 4) of substrate(s) 2 is deposited on step part(s) 9 and on substrate(s) 2 that remain exposed, for example, by a CVD method, a sputtering method, etc. A thickness of sidewall spacer-forming layer(s) 20 may be formed along a side face 9a of step part(s) 9 may be selected based on a desired width(s) W1 for a nanogap(s) NG. In other words, when forming nanogap(s) NG with narrow width(s) W1, a film thickness(es) of sidewall spacer-forming layer(s) 20 may be made small, whereas when forming nanogap(s) NG with a wide width(s) W1 may be formed, film thickness(es) of sidewall spacer-forming layer(s) 20 may be made large.

Next, insulating layer(s) 23 (mask layer[s]) overlaying sidewall spacer-forming layer(s) 20 may be formed, as shown in FIG. 9E, in which similar reference numerals are used to denote parts corresponding to those in FIG. 9C, and as shown in FIG. 9F, in which similar reference numerals are used to denote parts corresponding to those in FIG. 9D. A material of insulating layer(s) 23 that may be a mask layer, for example, silicon nitride (SiN), etc., which may be a same material as that of step part(s) 9, may be used. In some cases, insulating layer(s) 23 may be formed of an insulating material such as, for example, silicon nitride (SiN), which may be of a same insulating material as that of step part(s) 9, may be used as a mask layer(s). However mask layer(s) and step part(s) 9 may be formed of any material other than insulating material(s) may be used.

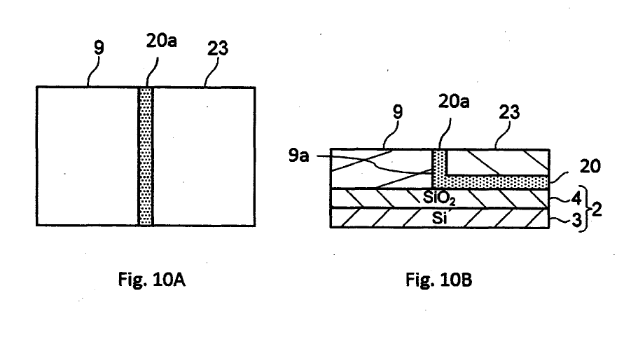

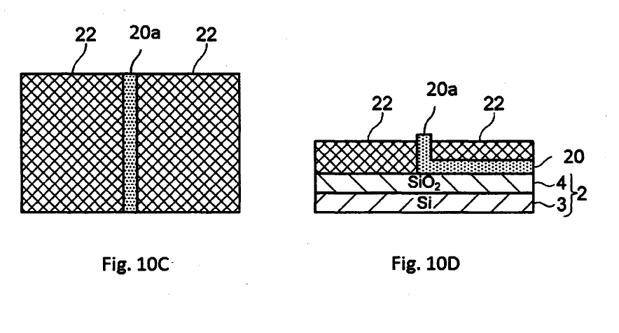

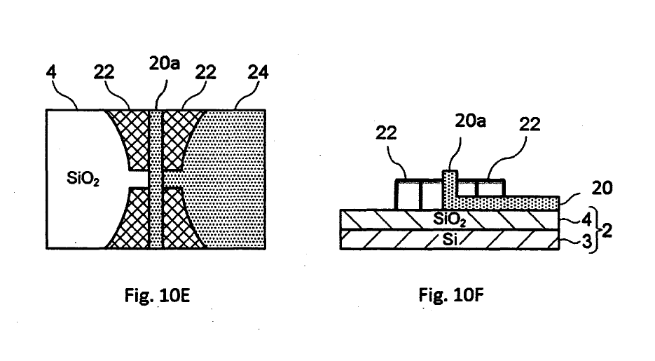

Next, as shown in FIG. 10A, in which similar reference numerals are used to denote parts corresponding to those in FIG. 9E, and as shown in FIG. 10B, in which similar reference numerals are used to denote parts corresponding to those in FIG. 9F, by overpolishing utilizing a planarizing process such as CMP, etc., a surface of step part(s) 9 and a surface of insulating layer(s) 23 may be exposed. Furthermore in some cases, from the sidewall spacer-forming layer(s) 20, only a top surface of sidewall spacer(s) 20a that may be provided alongside face 9a of the step part in an erect manner on the substrate(s) 2 may be exposed between step part(s) 9 and insulating layer(s) 23.

In some cases, the processing steps described above and shown in FIGS. 9A to 10B may describe a method for production of the sidewall spacer(s) 21. Sidewall spacer(s) 20a may thus be produced, which may be used for forming nanogap(s) NG (described later). Thus in some cases for methods for production of sidewall spacer(s) as described hereinabove, sidewall spacer(s) 11 may have a height of 1,000 nm or less (nanoscale), or 30 nm or less, and further may have a thickness of 2 nm or less, or 1 nm or less, as required according to intended use. Next, processing steps whereby nanogap(s) NG may be formed in an erect manner on substrate(s) 2 using such a sidewall spacer(s) 20a and production of nanogap electrode device(s) 31 of FIG. 8 are described herein below.

Step part(s) 9 and insulating layer(s) 23 may then be removed by etching so that silicon oxide layer(s) 4 and sidewall spacer-forming layer(s) 20 may be exposed (not shown). Subsequently, a resist coating agent may be applied onto silicon oxide layer(s) 4 and sidewall spacer-forming layer(s) 20, and may be cured to form resist layer(s) 22, as shown in FIG. 10C, in which similar reference numerals are used to denote parts corresponding to those in FIG. 10A, and as shown in FIG. 10D, in which similar reference numerals are used to denote parts corresponding to those in FIG. 10B.

In some cases for a production method of sidewall spacer(s) 21, sidewall spacer(s) 20a may be formed in an erect manner from sidewall spacer-forming layer(s) 20 so that they create L-shaped cross section. Erected sidewall spacer(s) 20a may be supported by the remaining part of sidewall spacer-forming layer(s) 20. Thus, even if sidewall spacer(s) 20a may be subjected to a load from resist coating agent(s) when resist coating agent(s) may be applied, load placed on sidewall spacer(s) 20a may be received by sidewall spacer-forming layer(s) 20, and thus, it may be possible to prevent sidewall spacer(s) 20a from falling, being inclined, or being deformed.

Next, regions of resist layer(s) 22 at which first electrode(s) 5 and second electrode(s) 6 may be formed may be removed by a photolithographic technique so that resist layer(s) 22 may have a pattern formed therein. Thus, a surface of substrate(s) 2 (silicon oxide layer(s) 4) may be exposed at regions at which first electrode(s) may be later formed, and sidewall spacer-forming layer(s) 20 may be exposed at regions at which second electrodes may be later formed, as shown in FIG. 10E, in which similar reference numerals are used to denote parts corresponding to those in FIG. 10C, and as shown in FIG. 10F, in which similar reference numerals are used to denote parts corresponding to those in FIG. 10D.

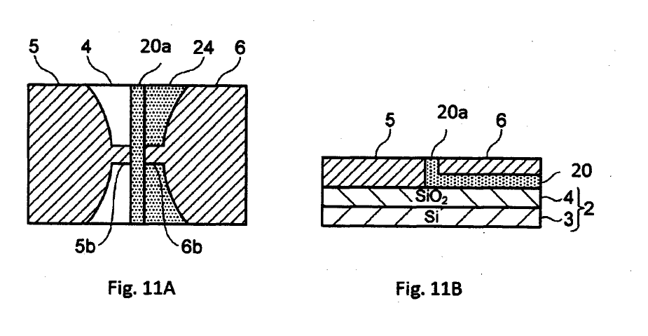

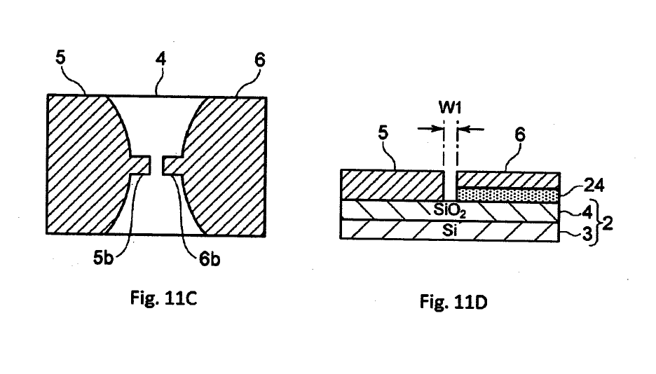

Next, a metal layer(s) is formed on silicon oxide layer(s) 4 that may be exposed at regions at which first electrode(s) 5 may be formed, on sidewall spacer-forming layer(s) 20 exposed at region(s) at which second electrode(s) 6 may be formed, on resist layer(s) 22 as an electrode-forming mask that remains at a region other than those at which first electrode(s) 5 and second electrode(s) 6 may be formed, and on sidewall spacer(s) 21. After that, patterned resist layer(s) 22 (electrode-forming mask) may be subjected to a photolithographic lift off process to remove metal layer(s) on resist layer(s) 22. Thus, first electrode(s) 5 and second electrode(s) 6 with electrode tip portions 5b and 6b facing each other across sidewall spacer(s) 20a may be formed on substrate(s) 2 as shown in FIG. 11A, in which similar reference numerals are used to denote parts corresponding to those in FIG. 10E, and as shown in FIG. 11B, in which similar reference numerals are used to denote parts corresponding to those in FIG. 10F. At this stage, sidewall spacer(s) 20a remain(s), and lower space(s) 24 remains at a region covered by patterned resist layer(s) 22. Metal layer used here may be material(s) with different etching rate(s) than lower spacer(s) 24.

Finally, as shown in FIG. 11C, in which similar reference numerals are used to denote parts corresponding to those in FIG. 11A, and as shown in FIG. 11D, in which similar reference numerals are used to denote parts corresponding to those in FIG. 11D, part of sidewall spacer-forming layer(s) 20 and sidewall spacer(s) 21, may be removed by a directional process, for example, by dry etching, creating lower spacer 24 between substrate(s) 2 and second electrode(s) 6. Thus, nanogap(s) NG, having a same width(s) W1 as that of sidewall spacer(s) 21, may be formed between second electrode(s) 6 and first electrode(s) 5. Accordingly, nanogap electrode device(s) as shown in FIG. 8 can be produced.

As described above, in some cases for methods of producing a nanogap electrode devices, after sidewall spacer-forming layer(s) 20 may be provided on step part(s) 9 that may be formed on predetermined region(s) on substrate(s) 2, and on substrate(s) 2 that remain(s) exposed, insulating layer(s) 23 that overlies sidewall spacer-forming layer 20 may be formed. Furthermore, in some cases for production methods, a planarizing process may be used to remove a part of insulating layer(s) 23 and to remove at least a part of sidewall spacer-forming layer(s) 20 that may be formed on step part(s) 9 adjoining sidewall spacer-forming layer(s) 20. Sidewall spacer(s) 20a may be formed between step part(s) 9 and insulating layer(s) 23 by making sidewall spacer-forming layer(s) 20 remain between step part(s) 9 and insulating layer(s) 23, and forming lower spacer(s) 24 between substrate(s) 2 and insulating layer(s) 23 by making sidewall spacer-forming layer(s) 20 remain between substrate(s) 2 and insulating layer(s) 23.

Then, step part(s) 9 and insulating layer(s) may be removed, and sidewall spacer(s) 21, integrally formed with sidewall spacer-forming layer(s) 20, may be provided in an erect manner on substrate(s) 2. Subsequently, first electrode(s) 5 and second electrode(s) 6 facing each other across sidewall spacer(s) 11 may be formed on substrate(s) 2, using patterned resist layer(s) 22 as an electrode-forming mask(s). Finally, after patterned resist layer(s) 22 may be removed, sidewall spacer(s) 20a and a part of sidewall spacer-forming layer(s) 20 that have been overlain with resist layer 22 may be removed, so that lower spacer(s) 24 is made to remain only between substrate(s) 2 and second electrode(s) 6. In this way, nanogap(s) NG, having a same width(s) W1 as that of sidewall spacer(s) 21, may be formed between first electrode(s) 5 and pair(s) of second electrode(s) 6 and lower spacer(s) 24.

As described hereinabove, in some cases for a method of production of a nanogap electrode device, nanogap(s) NG having a desired width(s) W1 may be formed by adjusting film thickness(es) of sidewall spacer(s) 21, and film thickness(es) of sidewall spacer(s) 20a may be formed so as to be very thin. Therefore, nanogap(s) NG having very small width(s) W1, corresponding to width(s) W1 of sidewall spacer(s) 21, may also be formed.

In view of the above, in some cases, lower spacer(s) 24 extending in a surface direction of substrate(s) 2, and sidewall spacer(s) 20a provided in an erect manner at an end of sidewall spacer-forming layer(s) 20, may be formed, and subsequently, patterned resist layer(s) 22 may be used to form first electrode(s) 5 on substrate(s) 2 and to form second electrode(s) 6 on sidewall spacer-forming layer(s) 20 so that second electrode(s) 6 may be arranged so as to be opposite to first electrode(s) 5 on an adjoining side of sidewall spacer(s) 21. Then, after removing patterned resist layer(s) 22, exposed sidewall spacer-forming layer(s) 20 may be removed so as to make lower spacer(s) 24 remain only between substrate(s) 2 and second electrode(s) 6, and may remove sidewall spacer(s) 20a so that nanogap(s) NG, having width(s) W1, adjusted by film thickness(es) of sidewall spacer(s) 21, may be formed between first electrode(s) 5 and second electrode(s) 6, and between first electrode(s) 5 and the lower spacer(s) 24. Thus, by adjusting film thickness(es) of sidewall spacer(s) 21, nanogap(s) NG having a same width(s) W1 as that of conventionally formed nanogap(s) may be formed, and even nanogap(s) NG having width(s) W1 that may be substantially narrower than conventionally formed nanogap width(s), may be formed.

Furthermore, in some cases for methods of producing sidewall spacer(s) 21, after layer-like sidewall spacer-forming layer(s) 20 is provided on step part 9 that may be formed on predetermined region(s) on substrate(s) 2, and provided on substrate(s) 2 that remain(s) exposed, insulating layer(s) 23 (mask layer[s]) that overlies sidewall spacer-forming layer(s) 20, may be formed. In addition, in some cases for methods for production of sidewall spacer(s) 21, a planarizing process may be used to remove a part of insulating layer(s) 23 and to remove sidewall spacer-forming layer(s) 20 at least at a part formed on step part(s) 9. Thus, sidewall spacer-forming layer(s) 20 may be made to remain between step part(s) 9 and insulating layer(s) 23 so that sidewall spacer(s) 21, provided in an erect manner, may be formed between step part(s) 9 and insulating layer(s) 23, and sidewall spacer-forming layer(s) 20 may be made to remain between substrate(s) 2 and insulating layer(s) 23 so that lower spacer(s) 24 may be formed between substrate(s) 2 and insulating layer(s) 23.

In some cases, step part(s) 9 and insulating layer(s) 23 may be removed, and first electrode(s) 5 and second electrode(s) 6 may be formed on opposite sides of sidewall spacer(s) 20a on substrate(s) 2 by use of resist layer(s) 22. Subsequently, resist layer(s) 22, sidewall spacer(s) 21, and sidewall spacer-forming layer(s) 20 may be removed, so that nanogap(s) NG having width(s) W1 of sidewall spacer(s) 20a may be formed between first electrode(s) 5 and second electrode(s) 6. Thus, in some cases for methods for production of sidewall spacer(s) 21, unlike in a conventional formation technique for forming a slot-like nanogap in the surface of an electrode layer by etching the electrode layer exposed from an opening in a metal mask, sidewall spacer(s) 20a by which nanogap(s) NG may be formed without the use of a conventional metal mask may be produced.

In some cases, slot-like gap(s) may be formed in silicon oxide layer(s) 4 below nanogap(s) NG by removing a part of a surface of silicon oxide layer(s) 4 that may be an upper layer of substrate(s) 2 by the use of first electrode(s) 5 and second electrode(s) 6 as masks, after nanogap electrode device(s) 31, as shown in FIG. 1, may be formed. For nanogap electrode device(s) as described above, electric field(s) may be generated in a gap in silicon oxide layer(s) 4 below nanogap(s) NG. When single-stranded DNA molecule(s) pass(es) through a gap in silicon oxide layer(s) 4, a local conductance may change(s). In response thereto, values of current(s) flowing through first electrode(s) 5 and second electrode(s) 6 may change. Based on such change in values of current(s), bases that constitute single-stranded DNA molecule(s) may be identified.

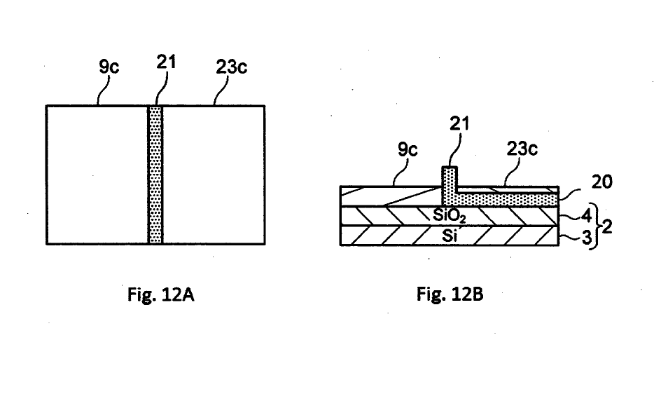

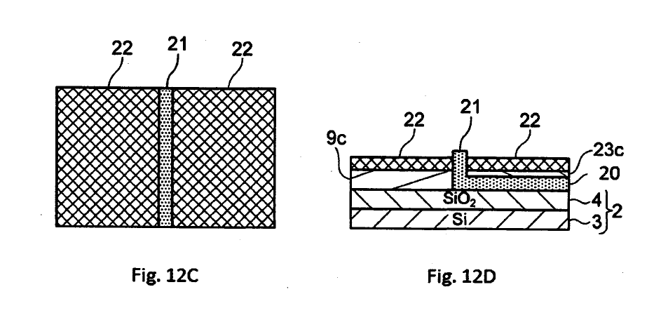

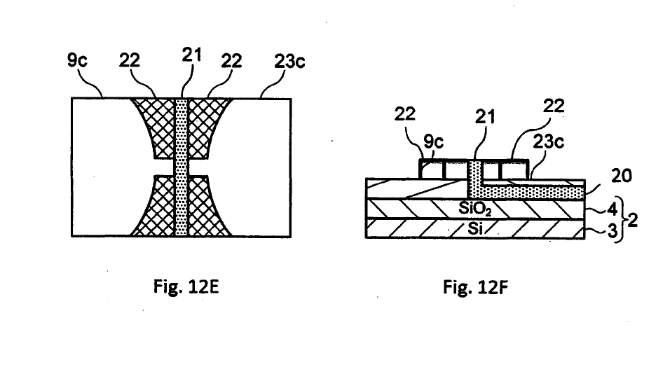

In some cases, there is described an example in which step part(s) 9 and insulating layer(s) 23 may all be removed so that a surface of substrate(s) 2 (silicon oxide layer(s) 4) and sidewall spacer-forming layer(s) 20 may be exposed. In some cases, as shown in FIG. 12A, in which similar reference numerals are used to denote parts corresponding to those in FIG. 10A, and as shown in FIG. 12B, in which similar reference numerals are used to denote parts corresponding to those in FIG. 10B, surfaces of step part(s) 9 and insulating layer(s) 23 may be partly removed so that step part(s) 9 and insulating layer(s) 23 may remain, thereby forming thin step part(s) 9c obtained by thinning step part(s) 9 and thin insulating layer(s) 23c may be obtained by thinning insulating layer(s) 23.

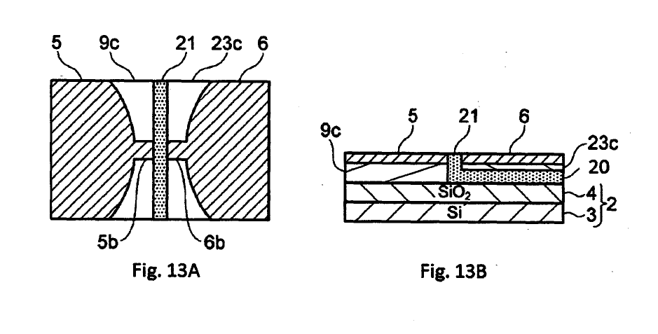

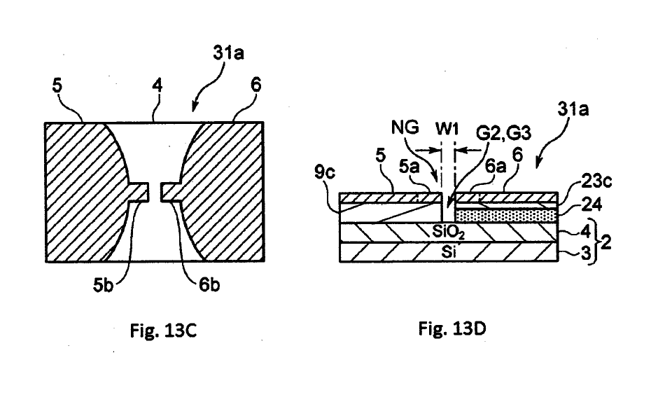

Unlike nanogap electrode device(s) 31 shown in FIG. 8, nanogap electrodes produced in this way (described later referring to FIGS. 13C and 13D) may have a configuration in which thin step part(s) 9c may be formed between substrate(s) 2 and first electrode(s) 5, and thin insulating layer(s) 23c may be formed between lower spacer 24 and second electrode(s) 6.

In some cases for a production method, surfaces of step part(s) 9 and insulating layer(s) 23 (mask layer[s]) as shown in FIG. 10A and FIG. 10B may be partly removed, so that thin step part(s) 9c an overlying surface of substrate(s) 2, and thin insulating layer(s) 23c overlying a surface of lower spacer 24, may be formed as shown in FIG. 12A and FIG. 12B. At this time, the surfaces of step part(s) 9 and insulating layer(s) 23 may be removed simultaneously and uniformly, so that thin step part(s) 9c and thin insulating layer(s) 23c, with surfaces thereof being aligned in height, may be formed. As shown in FIG. 12C, in which similar reference numerals are used to denote parts corresponding to those in FIG. 12A, and as shown in FIG. 12D, in which similar reference numerals are used to denote parts corresponding to those in FIG. 12B, a resist coating agent is applied onto thin step part(s) 9c and thin insulating layer(s) 23c, and may be cured to form resist layer(s) 22.

After that, a photolithographic technique may be used to remove resist layer(s) 22 at regions at which first electrode(s) 5 and second electrode(s) 6 may be formed, so that resist layer(s) 22 may have a pattern formed therein. Thus, thin step part(s) 9c may be exposed at a region at which first electrode(s) 5 may be formed, and thin insulating layer(s) 23c may be exposed at region(s) at which second electrode(s) 6 may be formed, as shown in FIG. 12E, in which similar reference numerals are used to denote parts corresponding to those in FIG. 12C, and as shown in FIG. 12F, in which similar reference numerals are used to denote parts corresponding to those in FIG. 12D.

Next, metal layer(s) may be deposited on thin step part(s) 9c that may be exposed at a region at which first electrode(s) 5 may be formed, on thin insulating layer(s) 23c that may be exposed at region(s) at which second electrode(s) 6 may be formed, and on resist layer(s) 22 as electrode-forming mask(s) that remain(s) at a region other than those at which first electrode(s) 5 and second electrode(s) 6 may be formed, and on sidewall spacer(s) 21. After that, patterned resist layer(s) 22 (electrode-forming mask[s]) may be removed to remove metal layer(s) on resist layer(s) 22 (using a lift-off process). Thus, first electrode(s) 5 and second electrode(s) 6 with electrode tip portions 5b and 6b facing each other across sidewall spacer(s) 11 may be formed on substrate(s) 2, as shown in FIG. 13A, in which similar reference numerals are used to denote parts corresponding to those in FIG. 12E, and as shown in FIG. 13B, in which similar reference numerals are used to denote parts corresponding to those in FIG. 12F. At this time, at regions from which resist layer(s) 22 may be removed, thin step part(s) 9c and thin insulating layer(s) 23c may be exposed.

Finally, exposed thin step part(s) 9c and exposed thin insulating layer(s) 23c which may exist between first electrode(s) 5 and second electrode(s) 6, lower spacer(s) 24 overlain with exposed thin insulating layer(s) 23c, and sidewall spacer(s) 20a may be removed by directional etching, for example, by dry etching, so that nanogap electrode device(s) 31a having nanogap(s) NG between electrode tip portion(s) 5b of first electrode(s) and electrode tip portion(s) 6b of second electrode(s) 6 may be formed, as shown in FIG. 13C, in which similar reference numerals are used to denote parts corresponding to those in FIG. 13A, and as shown in FIG. 13D, in which similar reference numerals are used to denote parts corresponding to those in FIG. 13B.

In some cases for production methods, surface(s) of lower spacer(s) 24 may be overlain with thin insulating layer(s) 23c that may be made to remain when resist layer(s) 22 may be formed, as shown in FIG. 12C and FIG. 12D. Thus, even if sidewall spacer(s) 20a may be pushed by a resist coating agent, lower spacer(s) 24 and thin insulating layer(s) 23c may not be damaged by force(s) applied to sidewall spacer(s) 21, so that it is accordingly possible to prevent sidewall spacer 21 from falling.

Furthermore in some cases, by adjusting a film thickness(es) of sidewall spacer(s) 20a during a production process, nanogap(s) NG having a same width(s) W1 as that of a conventionally formed nanogap can be formed, and even nanogap(s) NG having width(s) W1 that may be substantially narrower than a conventionally formed nanogap width may also be formed.

In some cases wherein step part(s) 9 may be formed on substrate(s) 2, and insulating layer(s) 23 may be formed on lower spacer(s) 24, as shown in FIG. 10A and FIG. 10B, whereby step part(s) 9 and insulating layer(s) 23 may be patterned without using a resist layer(s), and first electrode(s) 5 and second electrode(s) 6 may be formed using patterned step part(s) 9 and patterned insulating layer(s) 23.

In some cases, first step part(s) 9 may be formed on substrate(s) 2 (FIG. 9A and FIG. 9B), then sidewall spacer-forming layer(s) 20 and insulating layer(s) 23 may be formed (FIG. 9E and FIG. 9F), and subsequently, sidewall spacer(s) 20a may be provided in an erect manner on substrate(s) 2 between step part(s) 9 and insulating layer(s) 23 (FIG. 10A and FIG. 10B) using a planarizing process such as CMP, etc.

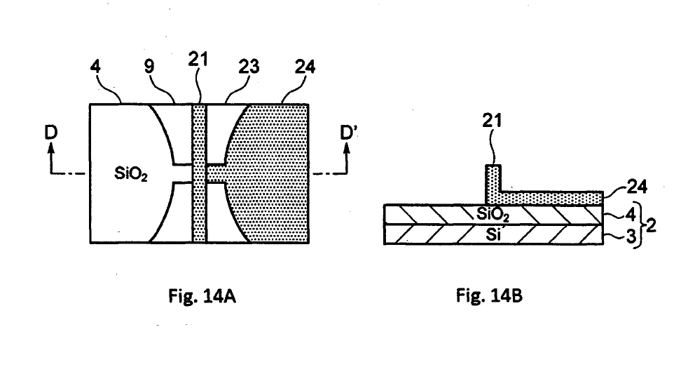

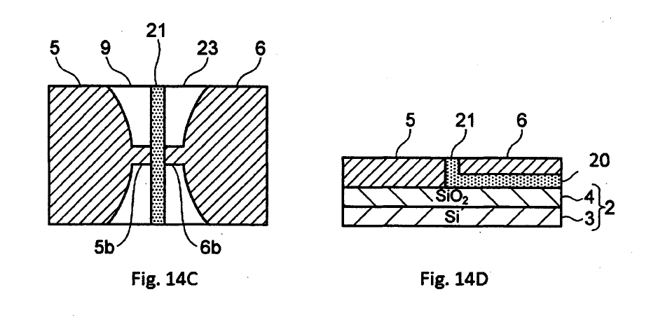

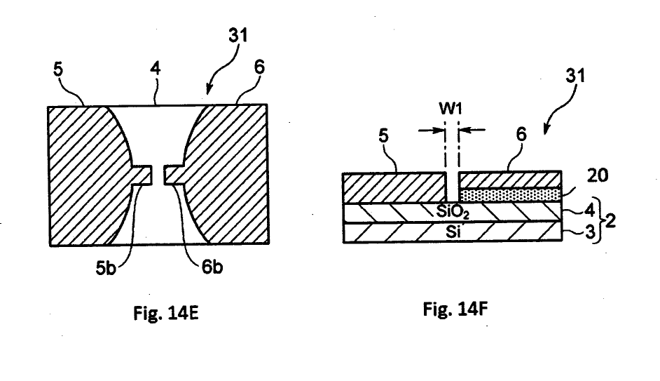

Next, after forming a layer-like resist mask on a surface of step part(s) 9, on a surface of sidewall spacer(s) 21, and on a surface of insulating layer(s) 23, which may be exposed, a photolithographic technique may be used to remove a part of step part(s) 9 and a part of insulating layer(s) 23 so that patterned step part(s) 9 and patterned insulating layer(s) 23 (electrode-forming masks) may be formed, as shown in FIG. 14A, in which similar reference numerals are used to denote parts corresponding to those in FIG. 10A, and as shown in FIG. 14B, illustrating a side sectional view taken along the line D-D′ in FIG. 14A. As shown in FIG. 14A and FIG. 14B, a pattern of a part to be removed from step part(s) 9 and a pattern of a part to be removed from insulating layer(s) 23 may correspond to a contour shape of first electrode(s) 5 and a contour shape of second electrode(s) 6 shown in FIG. 8, respectively. Thus, step part(s) 9 may be removed at regions at which first electrode(s) 5 may be formed, so that a surface (of silicon oxide layer(s) 4) of substrate(s) 2 may be exposed thereby. Insulating layer 23 may be removed at regions at which second electrode(s) 6 may be formed, so that lower spacer(s) 24 may be exposed thereby.

Next, metal layer(s) may be formed on silicon oxide layer(s) 4 exposed at regions at which first electrode(s) 5 and on lower spacer(s) 24 exposed at regions at which second electrode(s) 6, may be formed, and on step part(s) 9 and insulating layer(s) 23 remaining at regions other than regions at which first electrode(s) 5 and second electrode(s) 6 may be formed. Subsequently, a planarizing process, for example, CMP, etc., may be performed so that a surface of remaining step part(s) 9, a surface of remaining insulating layer(s) 23, and a surface of sidewall spacer(s) 20a may all be exposed. As a result, as shown in FIG. 14C, in which similar reference numerals are used to denote parts corresponding to those in FIG. 14A, and as shown in FIG. 14D, in which similar reference numerals are used to denote parts corresponding to those in FIG. 14B, metal layer(s) existing at regions of patterned step part(s) 9 and patterned insulating layer(s) 23 (electrode-forming masks) may be removed, so that step part(s) 9 and insulating layer(s) 23 may be exposed, and metal layer(s) existing at regions of sidewall spacer(s) 20a may be removed, so that sidewall spacer(s) 20a may be exposed, so that first electrode(s) 5 and second electrode(s) 6 with electrode tip portions 5b and 6b facing each other across sidewall spacer(s) 20a may be formed on substrate(s) 2.

Next, step part(s) 9 and insulating layer(s) 23, which may be exposed, may be etched off, so that silicon oxide layer(s) 4 may be exposed state at regions at which step part(s) 9 between first electrode(s) 5 and sidewall spacer(s) 20a previously existed, and lower spacer(s) 24 may be exposed at regions at which insulating layer(s) 23 between second electrode(s) 6 and sidewall spacer(s) 20a previously existed, as shown in FIG. 14C and FIG. 14D. In some cases, sidewall spacer(s) 20a and exposed lower spacer(s) 24 may be removed by directional etching, for example by dry etching, so that nanogap electrode device(s) 31 having nanogap(s) NG with a same width(s) W1 as that of sidewall spacer(s) 20a may be formed between electrode tip portions 5b and 6b, as shown in FIG. 14E, in which similar reference numerals are used to denote parts corresponding to those in FIG. 14C, and as shown in FIG. 14F, in which similar reference numerals are used to denote parts corresponding to those in FIG. 14D. Accordingly, nanogap electrode device(s) 31 as shown in FIG. 8 can be produced.

As described hereinabove, for some cases for methods of producing nanogap electrode device(s) 31, after sidewall spacer-forming layer(s) 20 may be provided on step part(s) 9 that may be formed on predetermined region(s) on substrate(s) 2, and provided on substrate(s) 2 that remain(s) exposed, insulating layer(s) 23 that overlies sidewall spacer-forming layer(s) 20 may be formed. In some cases for production methods, a planarizing process may be used to remove a part of insulating layer(s) 23 and to remove sidewall spacer-forming layer(s) 20 at least at a part formed on step part(s) 9 adjoining sidewall spacer-forming layer(s) 20, whereby sidewall spacer-forming layer(s) 20 is made to remain between step part(s) 9 and insulating layer(s) 23, so that sidewall spacer(s) 20a may be formed between step part 9 and insulating layer 23, and whereby sidewall spacer-forming layer(s) 20 may be made to remain between substrate(s) 2 and insulating layer(s) 23, so that lower spacer(s) 24 that extends in a surface direction of substrate(s) 2 may be formed between substrate(s) 2 and insulating layer(s) 23.

Then, step part(s) 9 and insulating layer(s) 23 may be patterned. Using patterned step part(s) 9 and patterned insulating layer(s) 23 as electrode-forming masks, first electrode(s) 5 and second electrode(s) 6 facing each other across sidewall spacer(s) 20a may be formed on substrate(s) 2. Finally, after removing remaining portions step part(s) 9 and remaining insulating layer(s) 23, lower spacer(s) 24 at regions that have been overlain with insulating layer(s) 23, and sidewall spacer(s) 20a may be removed, so that lower spacer(s) 24 may be made to remain only between substrate(s) 2 and second electrode(s) 6. Thus, nanogap(s) NG, having a same width(s) W1 as that of sidewall spacer(s) 21, may be formed between first electrode(s) 5 and pair(s) of second electrode(s) 6 and lower spacer(s) 24.

As described hereinabove, in some cases for methods of production of nanogap electrode device(s) 31, nanogap(s) NG having a desired width(s) W1 may be formed by adjusting a film thickness of sidewall spacer(s) 21. Film thickness of sidewall spacer(s) 21 may be be formed so as to be very thin, and nanogap(s) NG having a very small width(s) W1 corresponding to a width W1 of sidewall spacer(s) 20a may be also formed.

In view of the above, in some cases for methods of production, lower spacer(s) 24 extending in a surface direction of substrate(s) 2, and sidewall spacer(s) 20a provided in an erect manner at an end(s) of lower spacer(s) 24, may be formed, and subsequently, patterned step part(s) 9 and patterned insulating layer(s) may be used to form first electrode(s) 5 on substrate(s) 2 and to form second electrode(s) 6 on lower spacer(s) 24 so that second electrode(s) 6 may be arranged opposite to first electrode(s) 5 across sidewall spacer(s) 21. Then, after removing patterned step part(s) 9 and patterned insulating layer(s) 23, exposed lower spacer(s) 24 may be removed so that lower spacer(s) 24 only remains between substrate(s) 2 and second electrode(s) 6, and sidewall spacer(s) 20a may be removed so that nanogap(s) NG, having a width(s) W1 adjusted by film thickness(es) of sidewall spacer(s) 21, may be formed between first electrode(s) 5 and second electrode(s) 6, and between first electrode(s) 5 and lower spacer(s) 24. Thus, by adjusting a film thickness of sidewall spacer(s) 21, nanogap(s) NG having a same width(s) W1 as that of a conventionally formed nanogap can be formed, and even a nanogap(s) NG having a width(s) W1 substantially narrower than a conventionally formed nanogap width may be formed.

In some cases, after nanogap electrode device(s) 31 as shown in FIG. 14E and FIG. 14F may be formed, a slot-like gap may be additionally formed in silicon oxide layer(s) 4 below nanogap(s) NG by partly removing a surface of silicon oxide layer(s) 4 that may be a surface of substrate(s) 2 by the use of first electrode(s) 5 and second electrode(s) 6 as masks. For nanogap electrode device(s) as described hereinabove, electric field(s) may be generated in a gap in silicon oxide layer(s) 4 below nanogap(s) NG. When single-stranded DNA molecule(s) passes through a gap in silicon oxide layer(s) 4, the local conductivity may change(s). In response thereto, values of current(s) flowing through first electrode(s) 5 and second electrode(s) 6 may change. Based on such changes in values of current(s), bases that constitute single-stranded DNA molecule(s) may be identified.

In some embodiments, after forming of first electrode 5 and second electrode 6, but before removal of sidewall spacer 11, a lift off process may utilize to remove metal which may have been deposited on patterned resist layer(s) 12, while leaving metal deposited to form first electrode 5, second electrode 6 and sidewall spacer 11. A dielectric layer (not shown) may be deposited, which may cover electrode 5, second electrode 6, sidewall spacer 11 and regions of silicon oxide layer 4 which may be exposed as a result of the removal of patterned resist layer(s) 12. A thickness of said dielectric layer may be the same as the thickness of first electrode 5 and second electrode 6, or may be less thick than a thickness of first electrode 5 and second electrode 6, or may be more thick than a thickness of first electrode and second electrode.

A portion of said dielectric layer which may have been applied over first electrode 5, second electrode and sidewall spacer may be polished using a CMP technique or other appropriate planarization methods, such that a thickness of first electrode 5, second electrode 6, and dielectric material which may have been deposited in regions formerly covered by patterned resist layer(s) 12 may have a same thickness.

Sidewall spacer 11 may then be removed, leaving a channel (not shown) between first electrode 5 and second electrode 6, which may further extend between portions of dielectric layer (not shown) which may have been placed and may remain in locations where patterned resist layer(s) 12 may have been placed upon silicon oxide layer 4 which became exposed when patterned resist layer(s) 12 was removed. Said portions of dielectric layer which form portions of said channel, and electrode tip portions 5b and 6b which may face each other, may be coplanar as a result of sharing a same surface of sidewall spacer 11. As a result said channel may have smooth planar surfaces as said channel extends from between electrode 5 and electrode 6 to portions of dielectric which may form extension of said channel. Similarly, a top of said channel may be planar as a result of planarization using CMP or other processes, allowing a top, which may be a PDMS top or a top which may be affixed using an adhesive to be applied without causing changes in cross section of said channel due to differences in height of different regions of said channel, and which may adhere well as a result of said planarization removing irregularities in height. A width of said channel may be uniform, being the same as a width of sidewall spacer 11, and may be a same width between electrode tip portions 5b and 6b, and between portions of dielectric layer (not shown) which may be utilized to form said channel.

Said method of forming a channel whereby electrodes may be formed on either side of sidewall spacer 11, while regions adjacent to sidewall spacer and adjacent to first electrode 5 and second electrode 6 may have patterned resist or other means to create first electrode 5 and second electrode 6 may have a dielectric layer placed therein so as to create a channel which extends from between first electrode 5 and second electrode 6 in a manner such that a top of said elements may be planar as a result of said planarization, and walls of said channel may extend coplanar with electrode tip portions 5b and 6b may be utilized in combination with methods associated with FIGS. 2-13 to effectuate such structures.

In some cases for methods of forming sidewall spacer(s) as explained hereinabove, examples are described in which sidewall spacer(s) 11, 21, may be sandwiched between electrode tip portion(s) 5b of first electrode(s) 5 and electrode tip portion(s) 6b of second electrode(s) 6 may be linearly extended on substrate(s) 2, for example, as shown in FIG. 3A, FIG. 5C, FIG. 7A, and FIG. 10A. In some cases, one or more bent portions may be provided for bending sidewall spacer(s), which may be sandwiched between electrode tip portion(s) 5b of first electrode(s) 5 and electrode tip portion(s) 6b of second electrode(s) 6 in a direction bent so that it extends on substrate 2, in a manner so that sidewall spacer(s) extending between electrode tip portions(s) 5b and 6b in one direction may be bent to extend in another different direction from a one direction.

In this way, by forming a bent portion at a part of the sidewall spacer(s), even if an external force is applied, external force may be received by bent portion(s) so that sidewall spacer(s) may be supported. Thus, it is possible to maintain sidewall spacer(s) in an erect manner on a substrate, so that deformation or failure of sidewall spacer(s) 20a may be prevented.

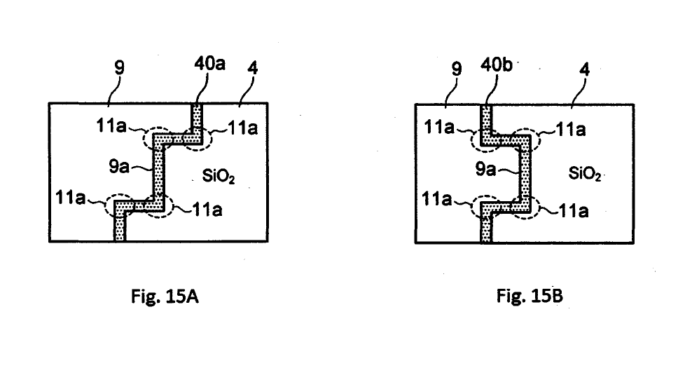

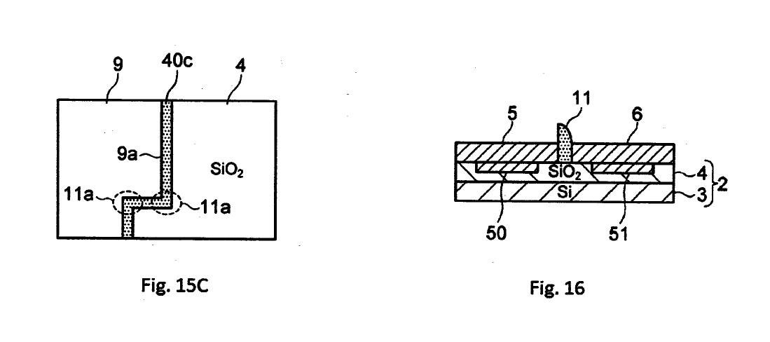

For example, sidewall spacers having such bent portions may be crank shaped sidewall spacers, horizontal U-shape sidewall spacers, and L-shape sidewall spacers, as viewed from a top (z-direction, FIG. 1). FIG. 15A shows an example of a crank-shaped sidewall spacer 40a as viewed from top. FIG. 15B shows an example of a horizontal U-shaped sidewall spacer 40b as viewed from top. FIG. 15C shows an example of an L-shaped sidewall spacer 40b as viewed from top. Each of the sidewall spacers 40a, 49b, and 40c shown in FIG. 15A, FIG. 15B, and FIG. 15C may have a structure including a plurality of bent portions 11a as viewed from top.

In some cases, side face(s) 9a of step part(s) 9 may be formed to have a crank shape, a horizontal U-shape, and an L-shape as viewed from the top by patterning step part(s) 9 formed on substrate(s) 2 in a desired shape. Then, sidewall spacer(s) 40a, 40b, 40c may be formed along side face(s) 9a, so that sidewall spacer(s) 40a, 40b, 40c including bent portions 11a consistent with a shape of side face(s) 9a may be formed as shown in FIG. 15A, FIG. 15B, and FIG. 15C. In some cases, there may be multiple bent portions 11a, some of which bend sidewall spacers 40a, 40b, 40c, which extend in one direction between the electrode tip portions 5b and 6b on the substrate 2, in a direction perpendicular to the one direction, so that sidewall spacers 40a, 40b, 40c may be maintained stably erect even when a resist coating agent is applied.

After sidewall spacer(s) 40a, 40b, 40c having bent portions may be formed, nanogap electrode device(s) 1 may be produced, for example, by processing steps shown and described in association with FIG. 3A to FIG. 4D, or by processing steps shown and described in association with FIG. 5A to FIG. 6F. By patterning so that sidewall spacer(s) 40a, 40b, 40c having bent portions 11a do not overlap tip portion(s) 5a of first electrode(s) 5 and tip portion(s) 6a of second electrode(s) 6, steps for forming first electrode(s) 5 and second electrode(s) 6 may not be affected, even if sidewall spacer(s) 40a, 40b, 40c have bent portions 11a.

A shape for sidewall spacer(s) 40a, 40b, 40c having bent portions 11a may not be limited to those shown in FIG. 15A, FIG. 15B, and FIG. 15C. For example, a bent portion to be provided may be a bent portion at which a sidewall spacer, which may be sandwiched between electrode tip portion 5b of first electrode 5, and may be bent in a manner so that sidewall spacer(s) extending between electrode tip portions 5b and 6b in one direction may be bent to extend in another direction different from a one direction. Sidewall spacer(s) having bent portions may be formed in an E-shape, an F-shape, a vertical U-shape, a T-shape, a curved shape such as a C-shape, or any other shape as viewed from the top. In some cases, width(s) of sidewall spacer(s) may also be selected depending on desired nanogap(s) NG width(s).

In some cases which include adjusting a film thickness of sidewall spacer(s) 40a, 40b, 40c, during a production process, nanogap(s) NG having a same width(s) W1 as that of a conventionally formed nanogap may be formed, and even nanogap(s) NG having width(s) W1 that may be substantially narrower than a conventionally formed nanogap width may be formed.

Alternatively or additionally, as materials for first and second electrodes 5 and 6, substrate(s) 2, sidewall spacer(s) 11, 21, etc., various materials may be used. Furthermore, shape(s) of first and second electrodes 5 and 6 may be any shape.

For some cases described hereinabove, nanogap electrode device(s) 1 are described which allows single-stranded DNA molecule(s) to pass through nanogap(s) NG between first electrode(s) 5 and second electrode(s) 6, and utilizes an ammeter to measure values of current(s) flowing through first electrode(s) 5 and second electrode(s) 6 when each base of single-stranded DNA molecule(s) passes through nanogap(s) NG between first electrode(s) 5 and second electrode(s) 6. Alternatively or additionally, nanogap electrode device may be applied to various other applications, including measurement of RNA molecules, double stranded DNA molecules, peptides or proteins, or other biopolymers, or organic molecules.

In other cases as described hereinabove, nanogap electrode device(s) 31 are described which allows single-stranded DNA molecule(s) to pass through nanogap(s) NG between first electrode(s) 5 and second electrode(s) 6, and utilizes an ammeter to measure values of current(s) flowing through first electrode(s) 5 and pair(s) of second electrode(s) 6 and lower spacer(s) 24 when each base of single-stranded DNA molecule(s) passes through nanogap(s) NG. However, the present invention is not limited thereto. The nanogap electrode device may be used in various other applications.

Furthermore, for other cases as described hereinabove, example are given in which sidewall spacer(s) 11 may be formed to gradually increase in width from a top thereof to substrate(s) 2. Alternatively or additionally, sidewall spacer-forming layer(s) may not be formed in a conformal manner. Sidewall spacer-forming layer(s) may be formed to have different film thicknesses at different locations by changing the film deposition conditions (such as temperature, pressure, applied gas, flow rate, etc.). It is also possible to use sidewall spacer(s) that may be formed to gradually decrease in width from a top(s) to a substrate(s), or sidewall spacer(s) formed to have a maximum or minimum width(s) at various portions, for example, at a top position(s), at a center position(s) of the substrate(s), etc.

For some cases described hereinabove, examples are given of substrate(s) 2, which may comprise silicon oxide layer(s) 4, and silicon substrate(s) 3, without an electrode-forming layer(s). Alternatively or additionally, first electrode(s) 5 and second electrode(s) 6 facing each other across sidewall spacer(s) may be formed by previously forming electrode-forming layers 50, 51 so as to be embedded in the surface of substrate(s) 2 with a predetermined distance in-between, growing electrode-forming layers 50, 51 to extend from a surface of substrate(s) 2 and to abut to sidewall spacer(s) 11 as shown in FIG. 16. In some cases for forming first electrode(s) and second electrode(s) from electrode-forming layers 50 and 51, electrode-forming layers 50 and 51 may comprise TiN which may be formed using a CVD method, or electrode-forming layers 50 and 51 may comprise Ni which may be formed by plating, so that first electrode(s) 5 and second electrode(s) 6 facing each other across sidewall spacer(s) 11 can be formed.

In this case, cases for production methods explained for the above-described respective cases may be combined appropriately.

For example, for substrate(s) 2 for which electrode-forming layers 50 and 51 may be embedded in a surface, sidewall spacer(s) 11 may be provided along a side face of step part(s) 9 by etching back as shown in FIG. 2 and FIG. 3. Alternatively, as shown in FIG. 5, insulating layer(s) 23, and also step part(s) 9 and sidewall spacer(s) 11 may be polished by CMP so that sidewall spacer(s) 11 may be provided in an erect manner between step part 9 and insulating layer(s) 23. At this time, sidewall spacer(s) 11 may be formed to have a bent portion as described above.

In some cases in which pair(s) of separated electrode-forming layers 50 and 51 may be formed embedded in an insulating material, and sidewall spacer(s) 11 may be formed on substrate(s) 2 between pair(s) of electrode-forming layers 50 and 51, and subsequently electrode-forming layers 50, 51, which may be exposed from a surface of a substrate(s), may be made to grow until electrode-forming layers 50, 51 abut sidewall spacer(s) 11, so that first electrode(s) 5 and second electrode(s) 6 facing each other across sidewall spacer(s) 11 may be formed. Alternatively or additionally, first electrode(s) 5 and second electrode(s) 6 facing each other across sidewall spacer(s) 11 may be formed by protrudingly forming a pair(s) of separated electrode-forming layers on a surface on a substrate in advance, and making a sidewall spacer(s) on a surface(s) of a substrate(s) between a pair(s) of electrode-forming layer(s), and subsequently making the pair(s) of electrode-forming layer(s) exposed from a surface(s) of the substrate(s) grow until the pair(s) of electrode-forming layers abut the sidewall spacer(s).

In some cases, first electrode(s) and second electrode(s) may be formed by making metal materials grow, wherein the materials of first and second electrodes may be different from electrode-forming layers 50 and 51, on electrode-forming layers 50 and 51. In other cases, first electrode(s) and second electrode(s) may each have electrode region(s) formed of a different metal material(s) which may be formed by making grow metal material(s) that may be different metal material(s) from first electrode(s) and second electrode(s) on first electrode(s) and second electrode(s) respectively, for example, by a metal plating method.

In some cases, first electrode(s) and second electrode(s) may be formed by creating an electrode region(s) formed of metal(s), which may be different from that of a lower layer(s) which may be formed by enlarging an initial metal form, for example by plating, which may be utilize different metal(s) from that of first and second electrodes, on first electrode(s) and second electrode(s), before removing sidewall spacer(s).

For example, when a sidewall spacer(s) may be formed at an end(s) of a lower spacer(s), and a sidewall spacer(s) may be provided in an erect manner on a substrate(s), first electrode(s) and second electrode(s) facing each other across sidewall spacer(s) may be formed by forming an electrode-forming layer(s) on a substrate(s), forming another electrode-forming layer(s) on a lower spacer(s), and subsequently, enlarging electrode-forming layer(s) facing each other across sidewall spacer(s) until electrode-forming layers abut a sidewall spacer(s).

In some cases, first electrode(s) and second electrode(s) may be provided by replacing first electrode(s) and second electrode(s) which may have been initially formed of a predetermined metal material(s), for example, such as Ni, with a different metal material(s) such as gold, that may be different than the initial predetermined meal material(s) e.g. Ni, etc., using plating such as gold plating, before sidewall spacer(s) 11 may be removed.

For some cases as described hereinabove, a nanogap electrode device having a nanogap having a same width as that of a sidewall spacer between a first electrode and a second electrode may be produced by use of a conductive material capable of serving as an electrode. In some cases, a microstructure may be produced by forming a first process part and a second process part using an insulating material or other various materials, other than a conductive material, so that a nanogap having a same width as that of a sidewall spacer may be formed between a first process part and a second process part.

In some cases for forming a sidewall spacer, after forming a first process part and a second process part across a sidewall spacer, the sidewall spacer is removed, so that a nanogap having a same width as that of the sidewall spacer can be formed between the first process part and the second process part. Accordingly, a microstructure having a nanogap between a first process part and a second process part may also be produced.

In some cases for producing a microstructure, by replacing “first electrode” with “first process part” and “second electrode” with “second process part”, “nanogap electrode device” described hereinabove may be a “microstructure”, in each of the cases described hereinabove. An outline of the microstructures corresponding to the respective cases described above follows.

In some cases described hereinabove, according to one example of a method for production of a microstructure, first, a sidewall spacer may be provided in an erect manner on a substrate, then, a first process part and a second process part facing each other across the sidewall spacer may be formed using a patterned resist layer, and subsequently, the resist layer and the sidewall spacer may be removed so that a nanogap having a width, adjusted by a film thickness of the sidewall spacer, may be formed between the first process part and the second process part. Thus, for a microstructure, a gap, having a width that is the same as a sidewall spacer width, may be formed between a first process part and a second process part, so that a nanogap having a desired width can be formed by adjusting a film thickness of the sidewall spacer.

Such a sidewall spacer can be formed to have a very thin film thickness, so that a very small nanoscale (for example, 1000 nm or less) nanogap corresponding to a width of a sidewall spacer may be formed between a first process part and a second process part. Thus, a microstructure may be formed to have a nanogap having a width of, for example, 5 nm to 30 nm, 2 nm or less, or 1 nm or less, as required according to intended use, between the first process part and the second process part, by adjusting a film thickness of the sidewall spacer.

In some cases described hereinabove, a microstructure having a gap, having a width that may be the same as a sidewall spacer width, between a first process part and a second process part may also be produced utilizing a production method described herein. A microstructure having a gap, having a width that may be the same as a sidewall spacer width, between a first process part and a second process part, may also be produced according to a production method explained hereinabove.

In some cases, a microstructure may also be produced by a production method explained hereinabove wherein such microstructures may be different in configuration from other microstructures described hereinabove in that a lower spacer may be formed as a lower layer of a second process part, and a nanogap, having a width that may be the same as a sidewall spacer width, may be formed between the a process part and a second process part.

In some cases as shown in FIG. 17, a nanogap electrode device 101 may be configured such that electrode-forming substrate 106 which may be formed at least in part of an insulating material such as a silicon oxide (SiO2) may be formed on a silicon substrate 102. First electrode 1010 which may be formed of a material such as titanium nitride (TiN), and second electrode 1011 which may be formed of a same material such as titanium nitride (TiN) as that of first electrode 1010, may be embedded in a surface of electrode-forming substrate 106. First electrode 1010 may comprise a generally semicircular base portion 1010a, a band-like nanogap-forming portion 1010b that may be integrally formed with base portion 1010a at a center of an arc part thereof, and a first electrode side surface 1010c that may be formed flat like a wall at an end face of nanogap-forming portion 1010b. Second electrode 1011 and First electrode 1010 may be formed so as to be substantially left-right symmetrical with nanogap NG (described later) as a center. Similar to first electrode 1010, second electrode 1011 may comprise a generally semicircular base portion 1011a, a band-like nanogap-forming portion 1011b that may be integrally formed with base portion 1011a at a center of an arc part thereof, and a second electrode side surface 1011c that may be formed flat like a wall at an end face of nanogap-forming portion 1011b.

Nanogap-forming portion 1010b of first electrode 1010 and nanogap-forming portion 1011b of second electrode 1011 may be arranged so that wall-like first electrode side surface 1010c and wall-like second electrode side surface 1011c may face each other across nanogap NG having a nanoscale width W1 (for example, 1,000 nm or less. In some cases for producing a nanogap electrode device, nanogap electrode device 101 may be formed with nanogap NG having a width W1, for example, of 10 nm or less, 2 nm or less, or 1 nm or less, as required according to intended use.

In some cases, electrode-forming substrate 106 may have thereon a slot-like channel 107 that may be in communication with nanogap NG. In some cases, channel 107 may be a volume which may be comprised of a slot (not shown) provided in a surface of electrode-forming substrate 106. A channel may be formed so that an object to be measured such as a single-stranded DNA molecule can pass through from one end to another other end of said channel. Channel 107 may be formed as a space across which first electrode-forming face 103a, which may be formed like a wall, and second electrode-forming face 104a, which may be similarly formed like a wall, may be arranged to face each other while maintaining a constant distance therebetween. Channel 107 may have a configuration in which it extends along a center axis O between first electrode-forming face 103a and second electrode-forming face 104a.

Wall-like first electrode side surface 1010c of first electrode 1010 may be exposed next to first electrode-forming face 103a, and wall-like second electrode side surface 1011c of second electrode 1011 may be exposed next to a second electrode-forming face 104a that may be arranged to face first electrode-forming face 103a. Channel 107, in communication with nanogap NG which may be formed between first electrode side surface 1010c of first electrode 1010 and second electrode side surface 1011c of second electrode 1011. This allows an object to be measured, passing through channel 107, to be directly guided to, and to pass through, nanogap NG.

In some cases, first electrode side surface 1010c of first electrode 1010 and second electrode side surface 1011c of second electrode 1011 may be arranged to face each other, across center axis O of channel 107 as a center, while maintaining a constant distance therebetween, i.e., separated substantially in parallel to face each other, so that an object to be measured, passing through channel 107 along a center axis of channel 107, may flow directly to nanogap NG along center axis O.

In some cases, channel 107 may be linearly formed in a band-like shape, and nanogap NG may be provided midway from one end to another end thereof. Thus, in nanogap electrode device 101, when a solution containing a single-stranded DNA molecule, which may be an object to be measured, and may be supplied from one end of channel 107, an object to be measured may be delivered to another end of channel 107 passing through nanogap NG along center axis O, and may be discharged from channel 107.

In some cases, channel 107 may be configured to be linearly formed in a band-like shape, and nanogap NG may be provided midway from one end to another end thereof, may be adopted. Alternatively or additionally, a channel may extend in a curved shape, such as an S-shape, a C-shape, etc., and nanogap NG may be provided midway, or at some other position between one end to another end thereof.

In some cases, channel 107 may be formed between first electrode-forming face 103a and second electrode-forming face 104a, and nanogap NG may be formed between first electrode side surface 1010c of first electrode 1010 and second electrode side surface 1011c of second electrode 1011, and may be produced during a production process by removing a wall-like sidewall spacer (described later) which may have been formed between first electrode-forming face 103a and second electrode-forming face 104a, and which may have been formed between first electrode side surface 1010c of first electrode 1010 and second electrode side surface 1011c of second electrode 1011 in a contiguous manner. Thus, channel 107 and nanogap NG may be formed in a space wherein wall-like sidewall spacer may have previously existed.

Thus, first electrode-forming face 103a and second electrode-forming face 104a which may define channel 107, and first electrode side surface 1010c of first electrode 1010 and second electrode side surface 1011c of second electrode 1011 which may define nanogap NG may be formed like a wall conforming to a shape of a side surface of a removed sidewall spacer. Furthermore, first electrode-forming face 103a and second electrode-forming face 104a, which may define channel 107, and first electrode side surface 1010c of first electrode 1010 and second electrode side surface 1011c of second electrode 1011, which may define nanogap NG, and may be formed like a wall, may be formed by removing a sidewall spacer that extends along center axis O, while maintaining a constant thickness. Thus, first electrode-forming face 103a and second electrode-forming face 104a, as well as first electrode side surface 1010c and second electrode side surface 1011c, may be formed to extend in a single direction while maintaining a face-to-face arrangement and maintaining a constant distance therebetween so as to correspond to a thickness of a sidewall spacer. Furthermore, channel 107 and nanogap NG may be formed in a space that may be formed by removing a sidewall spacer that may extend like a band with a constant height, so that a depth of channel 107 and a depth of nanogap NG may be set to correspond to a constant height of a sidewall spacer. Thus, channel 107 and nanogap NG may be formed with a same depth.

For some cases for producing a nanogap electrode device, wherein an etching rate of first electrode-forming face 103a and second electrode-forming face 104a may be the same as that of first electrode side surface 1010c and second electrode side surface 1011c when removing a wall-like sidewall spacer, which may be formed between first electrode-forming face 103a and second electrode-forming face 104a and between first electrode side surface 1010c of first electrode 1010 and second electrode side surface 1011c of second electrode 1011 in a contiguous manner, first electrode-forming face 103a and first electrode side surface 1010c of first electrode 1010 may be formed to be contiguous and flush with each other, and second electrode-forming face 104a and second electrode side surface 1011c of second electrode 1011 may be formed so as to be contiguous and substantially flush with each other.

In other cases for a production process, wherein an etching rate of first electrode-forming face 103a and second electrode-forming face 104a may be different from that of first electrode side surface 1010c and second electrode side surface 1011c, when removing a wall-like sidewall spacer, which may be formed between first electrode-forming face 103a and second electrode-forming face 104a and between first electrode side surface 1010c of first electrode 1010 and second electrode side surface 1011c of second electrode 1011 in a contiguous manner, first electrode-forming face 103a and first electrode side surface 1010c of first electrode 1010 may be formed so as to be contiguous, but may not be flush with each other, so that a slight difference in level may be formed at a boundary therebetween. Furthermore, second electrode-forming face 104a and second electrode side surface 1011c of second electrode 1011 may also be formed so as to be contiguous, but may not be flush with each other, so that a slight difference in level may be formed at a boundary therebetween.

In other cases, channel 107 and nanogap NG, each of which may conform to a shape of a sidewall spacer, may be formed at a same time by removing the wall-like sidewall spacer, which may be formed between first electrode-forming face 103a and second electrode-forming face 104a, between first electrode side surface 1010c of first electrode 1010 and second electrode side surface 1011c of second electrode 1011 in a contiguous manner, during a production process. Accordingly, first electrode-forming face 103a and first electrode side surface 1010c of first electrode 1010 may be formed so as to be contiguous, and a difference in level at a boundary therebetween may be smaller than conventionally produced. Furthermore, second electrode-forming face 104a and second electrode side surface 1011c of second electrode 1011 may also formed so as to be contiguous, and a difference in level at a boundary therebetween may also be smaller than conventionally produced. Thus, a difference between a width of channel 107 and a width of nanogap NG may be reduced.

Accordingly, a difference in level at a boundary between first electrode-forming face 103a and first electrode side surface 1010c of first electrode 1010, and a difference in level at a boundary between second electrode-forming face 104a and second electrode side surface 1011c of second electrode 1011, may be made smaller, for example, compared with a length of first electrode side surface 1010c and a length of second electrode side surface 1011c both of which may extend along center axis O. Therefore, an object to be measured, which may flow through channel 107, may be smoothly fed to nanogap NG without being affected by changes in a flow rate which may be caused by difference(s) in level at boundaries with nanogap NG.

In some cases, for example, first electrode-forming face 103a and second electrode-forming face 104a may be formed so that channel 107 that may be formed therebetween has a width of 10 nm or less, and preferably, 2 nm or less. First electrode side surface 1010c and second electrode side surface 1011c may be formed so that a width of nanogap NG therebetween may be within +2 nm, or may be within +0.2 nm relative to a width of channel 107. In some cases, for example, after removing a sidewall spacer, first electrode-forming face 103a and second electrode-forming face 104a may be further etched so as to increase a width of channel 107.

In some cases, in which electrode-forming substrate 106 wherein channel 107 and nanogap NG may be formed on a surface thereof, a solution supply part (or fluid supply member) 108 that may be recessed in a generally square shape, although solution supply part may be of any other appropriate shape, and may be formed at one end of channel 107, and solution discharge part (or fluid discharge member) 109 having a same or different shape as that of solution supply part 108 may be formed at another end of channel 107. Solution supply part 108 may have a communication opening 108a between communication opening-forming side surfaces 103c and 104b, provided on a same plane so as to be flush with each other. An internal volume of solution supply part 108 may be in communication with channel 107 via communication opening 108a. Similarly, solution discharge part 109 may also have a communication opening 109a between communication opening-forming side surfaces 103c and 104b provided on a same plane so as to be flush with each other. An internal volume of solution discharge part 109 may be in communication with channel 107 via communication opening 109a. In this way, an internal volume of solution supply part 108 and an internal volume of solution discharge part 109 may be in communication with each other via channel 107, and may be formed so that an object to be measured within solution supply part 108 may be moved into solution discharge part 109 via nanogap NG and channel 107.

In some cases, solution supply part 108 and solution discharge part 109 may be formed to have a depth that may be the same as a depth of nanogap NG and a depth of channel 107, and a width that may be greater than that of channel 107. In this way, a region of solution supply part 108 may be large compared with channel 107. Thus, solution supply part 108 may be designed so that a supply pump (not shown) can be easily positioned thereto, and that a solution from the supply pump can be stored in solution supply part 108 and directly supplied therefrom to channel 107. Furthermore, a region of solution discharge part 109 may be large compared with channel 107. Thus, solution discharge part 109 may be designed so that a discharge pump (not shown) may be easily positioned thereat, and that a solution fed from channel 107 may be flowed by a discharge pump and discharged outside.

In some cases, U-shaped sidewall spacer 105 formed of silicon nitride (SiN), etc., may be embedded in a surface of electrode-forming substrate 106. Sidewall spacer 105 which may have been a part of a sidewall spacer which may have been used to form nanogap NG between first electrode 1010 and second electrode 1011 and channel 107 during a production process, and which was not removed during said production process and thereby remains. In some cases, sidewall spacer 105 may be configured so that one end face thereof may be provided between sidewall spacer exposure side surfaces 103f and 104c of solution supply part 108 such that they may be flush therewith and may be exposed thereat, and so that another end face thereof may be provided between sidewall spacer exposure side surfaces 103f and 104c of a solution discharge part such that they may be flush therewith and may be exposed thereat.

An above-described electrode-forming substrate 106 may have first electrode-embedded layer 103, which may be plate-like, and second electrode-embedded layer 104 that may be embedded in a recess (not shown) provided in a surface of first electrode-embedded layer 103. First electrode-embedded layer 103 may be formed of an insulating material such as a silicon oxide, and may be formed on silicon substrate 102. For first electrode-embedded layer 103, one side of an inner side surface of a recess in a surface thereof may be exposed as first electrode-forming face 103a by which channel 107 may be defined, and a part of a bottom surface of a recess may be exposed as a bottom surface 103b by which channel 107 may be defined. Furthermore, first electrode 1010 may be embedded in a surface of first electrode-embedded layer 103, first electrode side surface 1010c of nanogap-forming portion 1010b of first electrode 1010 may be exposed at first electrode-forming face 103a.

First electrode-embedded layer 103, and second electrode-forming face 104a, which may define channel 107 which may be provided along a peripheral surface of second electrode-embedded layer 104, may be arranged to face first electrode-forming face 103a while maintaining a constant distance therefrom. In this way, in electrode-forming substrate 106, first electrode-forming face 103a provided on first electrode-embedded layer 103 and second electrode-forming face 104a provided on a peripheral surface of second electrode-embedded layer 104 may be arranged to face each other across center axis O of channel 107 as a center while maintaining a constant distance therebetween, so that channel 107 may be formed. In other words, channel 107 may not be a slot part that is like a slot that is provided by simply cutting a surface of electrode-forming substrate 106. Channel 107 may be formed by a combination of different components, i.e., first electrode-embedded layer 103 and second electrode-embedded layer 104.

Above-described solution supply part 108 may include communication opening-forming side surfaces 103c and 104b between which communication opening 108a may provide communication with channel 107, a communication opening opposite side surface 103e may be arranged to face communication opening-forming side surfaces 103c and 104b, sidewall spacer exposure side surfaces 103f and 104c between which one end surface of sidewall spacer 105 may be exposed, and a sidewall spacer opposite surface 103d may be arranged to face sidewall spacer exposure side surfaces 103f and 104c, and a square region may be defined by communication opening-forming side surfaces 103c and 104b may be arranged on a single surface, communication opening opposite side surface 103e, and sidewall spacer exposure side surfaces 103f and 104c may be arranged on another single surface, and sidewall spacer opposite surface 103d. First electrode-embedded layer 103 may be exposed as a bottom surface of solution supply part 108 that may be a square-shaped recessed region surrounded by communication opening-forming side surfaces 103c and 104b, communication opening opposite side surface 103e, sidewall spacer exposure side surfaces 103f and 104c, and sidewall spacer opposite surface 103d.

Similar to solution supply part 108, first electrode-embedded layer 103 may be exposed as a bottom surface of solution supply part 109 that may be a square-shaped recessed region surrounded by communication opening-forming side surfaces 103c and 104b, communication opening opposite side surface 103e, sidewall spacer exposure side surfaces 103f and 104c, and sidewall spacer opposite surface 103d.

In some cases, among communication opening-forming side surfaces 103c and 104b, communication opening opposite side surface 103e, sidewall spacer exposure side surfaces 103f and 104c, and sidewall spacer opposite surface 103d of solution supply part 108, one communication opening-forming side surface 103c, communication opening opposite side surface 103e, one sidewall spacer exposure side surface 103f, and sidewall spacer opposite surface 103d may be formed along first electrode-embedded layer 103, whereas other communication opening-forming side surface 104b and other sidewall spacer exposure side surface 104c may be formed along a peripheral surface of second electrode-embedded layer 104 of solution supply part 108.

In some cases, second electrode-embedded layer 104 may be arranged to be flush with first electrode-embedded layer 103, in which communication opening-forming side surface 104b may be formed along a peripheral surface and may be formed in communication opening-forming side surface 103c, with communication opening 108a being interposed therebetween, so that second electrode-embedded layer 104 partly defines a side surface within solution supply part 108 together with first electrode-embedded layer 103. Furthermore, in second electrode-embedded layer 104, sidewall spacer exposure side surface 104c that may be formed to extend at a right angle from the communication opening-forming side surface 104b may be arranged to be flush with sidewall spacer exposure side surface 103f that may be formed in first electrode-embedded layer 103, and may define a part of a side surface in solution supply part 108 together with first electrode-embedded layer 103.

In some cases, second electrode-embedded layer 104, which may be formed of an insulating material such as a silicon oxide so as to have a generally quadrilateral shape. Then, two adjacent corners may be cut approximately in an L-shape, so that communication opening-forming side surface 104b and the sidewall spacer exposure side surface 104c may be formed to be arranged at a right angle. Second electrode-forming face 104a, which may define channel 107, may be formed between communication opening-forming side surface 104b located at one corner and another opening-forming side surface 104b located at another corner. Sidewall spacer 105 may be formed along three sides of second electrode-embedded layer 104, except for one side wherein second electrode-forming face 104a and communication opening-forming side surface 104b may be formed. Second electrode 1011 may be embedded in a surface of second electrode-embedded layer 104 at a region surrounded by sidewall spacer 105, and second electrode side surface 1011c of nanogap-forming part 1011b in second electrode-forming face 104a may be exposed at second electrode forming face 104a.

For a nanogap electrode device as described hereinabove, for example, when a solution containing a single-stranded DNA molecule may be fed to solution supply part 108 by a supply pump (not shown), etc., the solution containing a single-stranded DNA molecule may be supplied to channel 107 through communication opening 108a of solution supply part 108, may be flowed from channel 107 to solution discharge part 109 through other communication opening 109a, and may be discharged from solution discharge part 109 by a discharge pump, etc. When using nanogap electrode device 101, a solution containing a single-stranded DNA molecule may pass through channel 107, a DNA base in the single stranded molecule may pass through nanogap NG between first electrode 1010 and second electrode 1011.

With nanogap electrode device 101, when a voltage is applied across first electrode 1010 and second electrode 1011 by a power source (not shown), and a single-stranded DNA molecule may be flowed so as to pass through nanogap NG between first electrode 1010 and second electrode 1011, values of current flowing through first electrode 1010 and second electrode 1011 may be measured by an ammeter, and bases that comprise a single-stranded DNA molecule may be identified based on current values. At this time, by appropriately selecting a gap width of nanogap NG between first electrode 1010 and second electrode 1011, nanogap electrode device 101 may analyze a sample with high sensitivity.

In some cases for methods for production of a nanogap electrode device 101, firstly, a plate-like component (not shown) may be prepared such that a layer-like first process layer formed of silicon oxide may be deposited on an entire surface of a silicon substrate 102 by a CVD (Chemical Vapor Deposition) method, an ALD (Atomic Layer Deposition) method, a sputtering method, a thermal oxidation method, or other appropriate methods or processes.

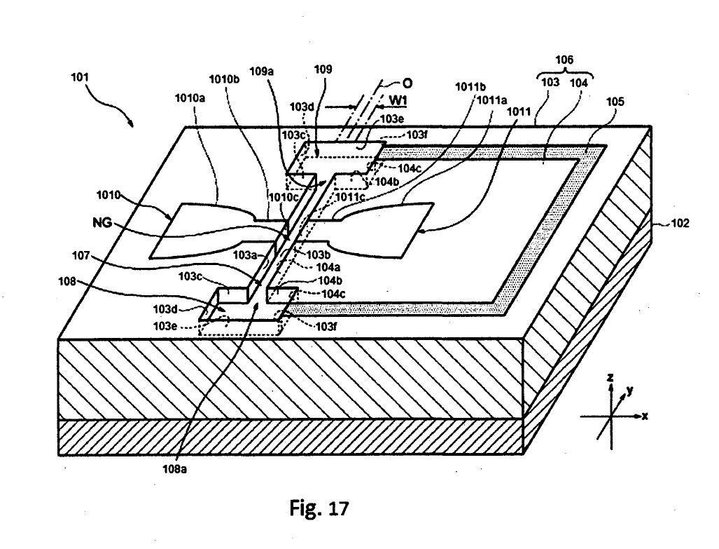

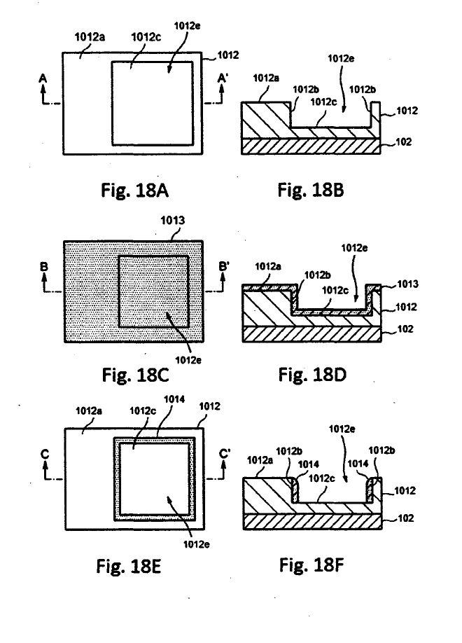

Then in some cases, as shown in FIG. 18A, and as shown in FIG. 18B illustrating a side sectional view taken along the line A-A′ in FIG. 18A, a surface of first process layer 1012 on silicon substrate 102 may be patterned using a photolithographic technique, so that a generally quadrilateral recess 1012e may be formed in a surface of first process layer 1012 at a predetermined location. In first process layer 1012, side surfaces 1012b are formed between surface 1012a and bottom surface 1012c of recess 1012e to extend by a distance corresponding to the depth of recess 1012e.

Then, as shown in FIG. 18C, in which similar reference numerals are used to denote parts corresponding to those in FIG. 18A, and as shown in FIG. 18D, illustrating a side sectional view taken along the line B-B′ in FIG. 18C, a layer-like sidewall spacer-forming layer 1013, which may be formed of an insulating material such as silicon nitride may be deposited on surface 1012a and in recess 1012e in first process layer 1012, for example, using a CVD method, an ALD method, a sputtering method, or any other appropriate method or process. As shown in FIG. 18D, layer-like sidewall spacer-forming layer 1013 may be deposited on surface 1012a of first process layer 1012 and on bottom surface 1012c in recess 1012e, and may also be deposited on side surfaces 1012b in recess 1012e. At this time, a film thickness of sidewall spacer-forming layer 1013 may be determined depending on a desired width W1 of nanogap NG and or a width of channel 107. In other words, when a nanogap NG with a small width W1 and a channel 107 with a small width so as to be consistent therewith, may be formed, a film thickness of sidewall spacer-forming layer 1013 may be made small, whereas when a nanogap NG with a large width W1 and a channel 107 with a large width so as to be consistent therewith may be formed, a film thickness of sidewall spacer-forming layer 1013 may be made large.

Then, as shown in FIG. 18E, in which similar reference numerals are used to denote parts corresponding to those in FIG. 18C, and as shown in FIG. 18F, illustrating a side sectional view taken along the line C-C′ in FIG. 18E, sidewall spacer-forming layer 1013 may be etched back to expose surface 1012a of first process layer 1012 and bottom surface 1012c in recess 1012e, so that sidewall spacer-forming layer 1013 may be made to remain only on side surfaces 1012b of first process layer 1012. Thus, a sidewall-like sidewall spacer 1014 may remain on side surfaces 1012b in recess 1012e.

Sidewall spacer 1014 may be formed along each of side surfaces 1012b corresponding to four sides of recess 1012e in first process layer 1012. In some cases, sidewall spacer 1014 may be formed like a sidewall so that it may taper as it extends toward its top.

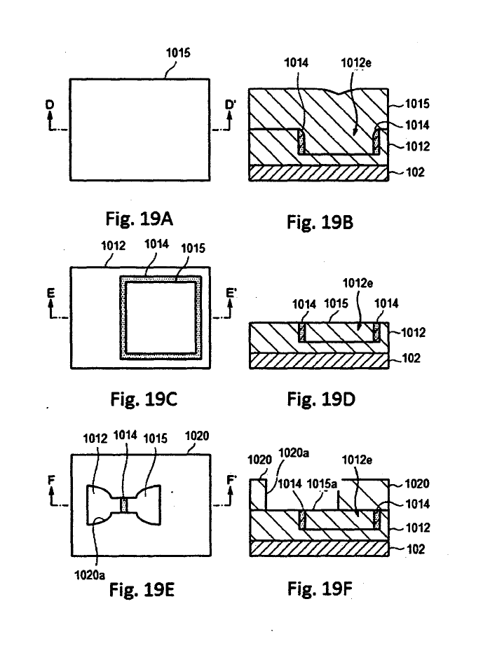

Then, as shown in FIG. 19A, in which similar reference numerals are used to denote parts corresponding to those in FIG. 18E, and as shown in FIG. 19B, illustrating a side sectional view taken along the line D-D′ in FIG. 19A, second process layer 1015, which may be formed of silicon oxide, etc., may be provided on sidewall spacer 1014 and first process layer 1012, for example, using a CVD method, an ALD method, a sputtering method, or any other method or process. In some cases an interior of recess 1012e may be filled with silicon oxide, and second process layer 1015 may thereby be formed.

Subsequently as shown in FIG. 19C, in which similar reference numerals are used to denote parts corresponding to those in FIG. 19A, and as shown in FIG. 19D, illustrating a side sectional view taken along the line E-E′ in FIG. 19C, a surface of the sidewall spacer 1014, a surface of first process layer 1012, and a surface of the second process layer 1015 may be subjected to planarization, for example, using a CMP (Chemical Mechanical Polishing) method, or any other appropriate method or process. By this planarization processing step, a surface of first process layer 1012 and a surface of sidewall spacer 1014 may be exposed, and a surface of second process layer 1015 may be exposed in recess 1012e in a region surrounded by sidewall spacer 1014. In some cases as shown in FIG. 19D, a surface of first process layer 1012, a surface of sidewall spacer 1014, and a surface of second process layer 1015 may be polished using CMP, until a tapered portion of sidewall spacer 1014 may be removed and sidewall spacer 1014 has a rectangularly-shaped cross section.

Next as shown in FIG. 19E, in which similar reference numerals are used to denote parts corresponding to those in FIG. 19C, and as shown in FIG. 19F, illustrating a side sectional view taken along the line F-F′ in FIG. 19E, a photoresist may be applied to a surface of first process layer 1012, a surface of sidewall spacer 1014, and a surface of second process layer 1015 to form a photoresist layer. Then, said photoresist layer may be patterned using a photolithographic technique. Thus, electrode-forming mask 1020 may be formed, in which opening parts 1020a, each corresponding to contour shapes of first electrode 1010 and second electrode 1011 (FIG. 17), may be patterned. From opening part 1020a patterned in electrode-forming mask 1020, first process layer 1012 and second process layer 1015, between which sidewall spacer 1014 may be sandwiched, may be exposed.

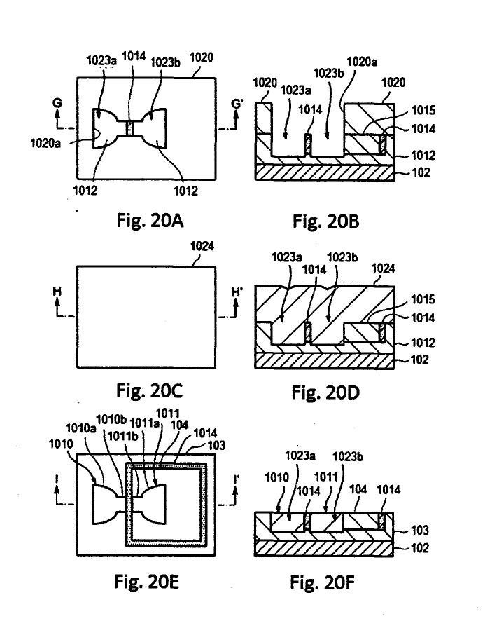

Subsequently as shown in FIG. 20A, in which similar reference numerals are used to denote parts corresponding to those in FIG. 19E, and as shown in FIG. 20B illustrating a side sectional view taken along the line G-G′ in FIG. 20A, a surface of first process layer 1012 and surface of second process layer 1015, which may be exposed through opening part 1020a of electrode-forming mask 1020, as a result of being etched, for example, by dry etching. By this processing step, a first electrode embedment recess 1023a may be formed in first process layer 1012 exposed through opening part 1020a, and second electrode embedment recess 1023b, which may be arranged opposite to first electrode embedment recess 1023a across sidewall spacer 1014, may be formed in second process layer 1015 exposed through opening part 1020a. Thus, sidewall spacer 1014 may be made to remain between first electrode embedment recess 1023a and second electrode embedment recess 1023b, so that sidewall spacer 1014 may be provided in an erect manner between first electrode embedment recess 1023a and second electrode embedment recess 1023b. Sidewall spacer 1014 remaining between first electrode embedment recess 1023a and second electrode embedment recess 1023b in this way may be provided in a manner so as to stand up on a surface of first process layer 1012 at a location exposed through opening part 1020a.

First electrode embedment recess 1023a, at which first electrode 1010 may be formed, may have a same contour shape as that of first electrode 1010 as shown in FIG. 17. Similarly, second electrode embedment recess 1023b, at which second electrode 1011 may be formed, may have a same contour shape as that of second electrode 1011 as shown in FIG. 17. In some exemplary cases as shown in FIG. 20B, in which a part of first process layer 1012 and a part of second process layer 1015 may be etched until each of side surfaces of sidewall spacer 1014 may be entirely exposed. Alternatively or additionally, first process layer 1012 and second process layer 1015 may be etched so that only a part of each side surface of sidewall spacer 1014 may be exposed.

In some cases as shown in FIG. 20A and FIG. 20B, second process layer 1015 exposed through opening part 1020a of electrode-forming mask 1020 may be entirely removed, so first process layer 1012 may be exposed at a bottom surface in second electrode embedment recess 1023b. Alternatively or additionally, second process layer 1015 may be provided on a bottom surface in second electrode embedment recess 1023b by partly leaving second process layer 1015 that is exposed through opening part 1020a of electrode-forming mask 1020.

Next as shown in FIG. 20C, in which similar reference numerals are used to denote parts corresponding to those in FIG. 20A, and as shown in FIG. 20D, illustrating a side sectional view taken along the line H-H′ in FIG. 20C, electrode-forming mask 1020 may be removed, and electrode layer 1024 which may be formed of titanium nitride or other appropriate materials, may be provided on a surface of first process layer 1012, a surface of sidewall spacer 1014, and a surface of second process layer 1015, for example using a CVD method, an ALD method, a sputtering method, or any other appropriate method or process. In some cases, as shown in FIG. 20D, a configuration, in which first electrode embedment recess 1023a and second electrode embedment recess 1023b may be filled with titanium nitride so that electrode layer 1024 may be formed therein, may be provided.

Subsequently, as shown in FIG. 20E, in which similar reference numerals are used to denote parts corresponding to those in FIG. 20C, and as shown in FIG. 20F, illustrating a side sectional view taken along the line I-I′ in FIG. 20E, a planarizing process may be utilized to polish a surface of electrode layer 1024, which may be polish first process layer 1012, sidewall spacer 1014 and second process layer 1015, for example, using a CMP method, until a top surface of sidewall spacer 1014 may be exposed, so that first electrode 1010 may be formed in first electrode embedment recess 1023a and second electrode 1011 may be formed in second electrode embedment recess 1023b.

Thus, first electrode-embedded layer 103, in which first electrode 1010 may be embedded in first electrode embedment recess 1023a in second process layer 1015, may be formed from first process layer 1012, and second electrode-embedded layer 104, in which second electrode 1011 may be embedded in second electrode embedment recess 1023b in second process layer 1015, may be formed from second process layer 1015. First electrode 1010 and second electrode 1011 which may be formed in this way may provide a configuration in which side surfaces of nanogap-forming portions 1010b and 1011b thereof may be arranged to face each other across sidewall spacer 1014.

Next as shown in FIG. 21A, in which similar reference numerals are used to denote parts corresponding to those in FIG. 20E, and as shown in FIG. 21B, illustrating a side sectional view taken along the line J-J′ in FIG. 21A, a photoresist may be applied to exposed surfaces of first electrode-embedded layer 103, second electrode-embedded layer 104, first electrode 1010, second electrode 1011, and sidewall spacer 1014, to form a photoresist layer. Then, a photolithographic technique may be used to form a pattern in said photoresist layer, thereby providing a reservoir forming mask 1025 in which solution reservoir aperture 1025a and discharge reservoir aperture 1025b may be patterned which may have contour shapes respectively conforming to solution supply part 108 and solution discharge part 109.

In some cases, solution reservoir aperture 1025a and discharge reservoir aperture 1025b in reservoir forming mask 1025 may be formed so that two L-shaped corners of sidewall spacer may be exposed. In practice, solution reservoir aperture 1025a and discharge reservoir aperture 1025b which may be patterned in reservoir forming mask 1025, in addition to two L-shaped corners, which may be arranged to face each other, of sidewall spacer 1014, surfaces of first electrode-embedded layer 103 and second electrode-embedded layer 104 near each L-shaped corner may be exposed as well.

Subsequently as shown in FIG. 21C, in which similar reference numerals are used to denote parts corresponding to those in FIG. 21A, and as shown in FIG. 21D illustrating a side sectional view taken along the line K-K′ in FIG. 21C, surfaces of first electrode-embedded layer 103 and second electrode-embedded layer 104, exposed through solution reservoir aperture 1025a and discharge reservoir aperture 1025b of reservoir forming mask 1025, may be etched, for example, by dry etching. Thus, solution reservoir recess 1026 and discharge reservoir recess 1027 may be formed at regions exposed through solution reservoir aperture 1025a and discharge reservoir aperture 1025b. Since an L-shaped corner of sidewall spacer 1014 remains in each of solution reservoir recess 1026 and discharge reservoir recess 1027, solution reservoir recess 1026 and discharge reservoir recess 1027 may be divided by an L-shaped corner of sidewall spacer 1014, so that an inner part thereof can be divided into two volumes. Among the two volumes above, in one volume in which a side surface of first electrode-embedded layer 103 may be exposed, communication opening-forming side surface 103c, communication opening opposite side surface 103e, sidewall spacer exposure side surface 103f, and sidewall spacer opposite surface 103d, which are illustrated in FIG. 17, may be formed. In another volume from which a side surface of second electrode-embedded layer 104 may be exposed, the communication opening-forming side surface 104b and the sidewall spacer exposure side surface 104c, which are illustrated in FIG. 17, can be formed.

In some cases as shown in FIG. 21E, in which similar reference numerals are used to denote parts corresponding to those in FIG. 21C, and as shown in FIG. 21F illustrating a side sectional view taken along the line L-L′ in FIG. 21E, first electrode-embedded layer 103 and second electrode-embedded layer 104, which may be exposed through solution reservoir aperture 1025a and discharge reservoir aperture 1025b of the reservoir forming mask 1025, may be etched until a side surface of sidewall spacer 1014 may be entirely exposed. In some cases first electrode-embedded layer 103 and second electrode-embedded layer 104 may be etched until a side surface of the sidewall spacer 1014 is entirely exposed. Alternatively or additionally, first electrode-embedded layer 103 and second electrode-embedded layer 104 may be deeply etched to a level beneath a bottom level of sidewall spacer 1014, or first electrode-embedded layer 103 and second electrode-embedded layer 104 may be shallowly etched to a level above a bottom level of sidewall spacer 1014, so that a difference in level is formed at a bottom surface in solution reservoir recess 1026 and discharge reservoir recess 1027 when sidewall spacer 1014 is removed by a processing step (described below).

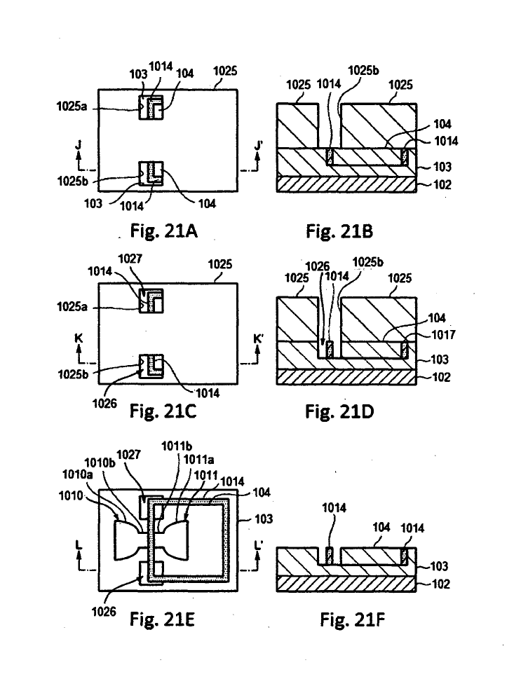

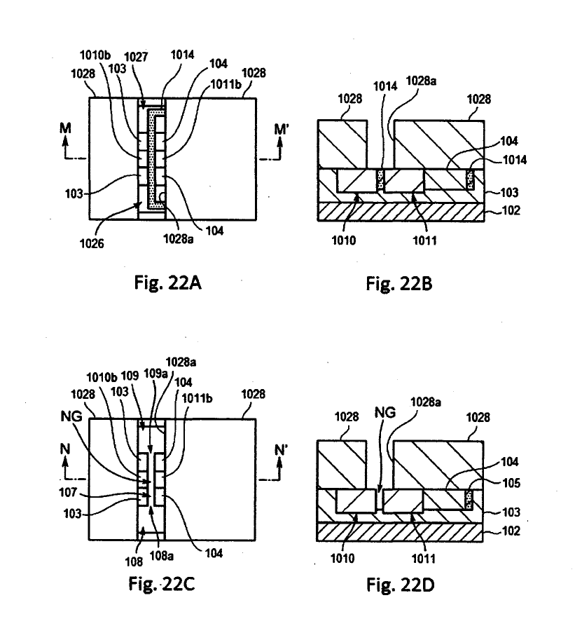

Next, as shown in FIG. 22A, in which similar reference numerals are used to denote parts corresponding to those in FIG. 21E, and as shown in FIG. 22B, illustrating a side sectional view taken along the line M-M′ in FIG. 22A, solution supply-discharge part-forming mask 1025 may be removed. Thereafter, a photoresist may be reapplied to surfaces of first electrode-embedded layer 103, second electrode-embedded layer 104, sidewall spacer 1014, first electrode 1010 and second electrode 1011, to form a photoresist layer. Next, said photoresist layer may be patterned using a photolithographic technique, so that nanogap forming mask 1028 in which opening part 1028a may be patterned may be formed. Through opening 1028a, linear sidewall spacer 1014 sandwiched between solution reservoir recess 1026 and discharge reservoir recess 1027, and L-shaped corners of sidewall spacer 1014 disposed in solution reservoir recess 1026 and discharge reservoir recess 1027, may be exposed.

In some cases opening part 1028a, which may be patterned in nanogap forming mask 1028 may be formed in a rectangular shape so that a width along a lateral direction may be substantially the same as that of solution reservoir recess 1026 and discharge reservoir recess 1027. First electrode-embedded layer 103, second electrode-embedded layer 104, nanogap-forming portion 1010b of first electrode 1010, and nanogap-forming portion 1011b of second electrode 1011 may be exposed at a region near sidewall spacer 1014, as well as sidewall spacer 1014.

Subsequently as shown in FIG. 22C, in which similar reference numerals are used to denote parts corresponding to those in FIG. 22A, and as shown in FIG. 22D, illustrating a side sectional view taken along the line N-N′ in FIG. 22C, sidewall spacer 1014 exposed through opening part 1028a of nanogap forming mask 1028 may be removed, for example, by dry etching. As a result, nanogap NG may be be formed at a region in which sidewall spacer 1014 between nanogap-forming portion 1010b of first electrode 1010 and nanogap-forming portion 1011b of second electrode 1011 may be removed. Furthermore, channel 107 having a same depth as that of nanogap NG may be formed at a region in which sidewall spacer 1014 between first electrode-embedded layer 103 and second electrode-embedded layer 104 may be removed. In some cases for production methods, channel 107 and nanogap NG, which may be in communication with channel 107, may be formed at the same time by merely removing sidewall spacer 1014.

Furthermore, when L-shaped corners of the sidewall spacer 1014 may be etched away, communication openings 108a and 109a are provided at boundaries with channel 107, so that solution supply part 108 and solution discharge part 109 having internal volumes, which are in communication with channel 107 via communication openings 108a and 109a, may be formed in solution reservoir recess 1026 and discharge reservoir recess 1027 (FIG. 22A and FIG. 22B). Sidewall spacer 1014 may be removed only at a region exposed through opening part 1028a of nanogap forming mask 1028, and a part of sidewall spacer 1014 may remain as a U-shape sidewall spacer 105 as shown in FIG. 17. Finally, nanogap forming mask 1028 may be removed, so that channel 107 may be formed at a region at which sidewall spacer 1014 existed between first electrode-forming face 103a of first electrode-embedded layer 103 and second electrode-forming face 104a of second electrode-embedded layer 104, and nanogap NG that may be in communication with channel 107 in a manner centered on the center axis O of channel 107 may be formed at a region at which sidewall spacer 1014 between first electrode side surface 1010c and second electrode side surface 1011c was removed, may be formed, as shown in FIG. 17.

In some cases, nanogap electrode device 101 may be provided with first electrode-embedded layer 103, which may be formed of an insulating material and may comprise first electrode-forming face 103a, and second electrode-embedded layer 104, which may also be formed of an insulating material and may comprise second electrode-forming face 104a. Furthermore, nanogap electrode device 101 may also comprise first electrode 1010 having first electrode side surface 1010c that may be exposed in first electrode-forming face 103a, and second electrode 1011 having second electrode side surface 1011c that may be exposed in second electrode-forming face 104a. Furthermore, nanogap electrode device 101 may comprise channel 107 and nanogap NG that may be in communication with channel 107. Channel 107 may be defined by first electrode-forming face 103a and second electrode-forming face 104a, which may be disposed in a face-to-face arrangement while maintaining a constant distance therebetween, and channel 107 may extend along center axis O between first electrode-forming face 103a and second electrode-forming face 104a.

Nanogap NG may be formed between first electrode side surface 1010c and second electrode side surface 1011c, which may be arranged to face each other across center axis O of channel 107 as a center, while maintaining a constant distance therebetween. In some cases, nanogap electrode device 101, first electrode-forming face 103a and first electrode side surface 1010c may be formed in a contiguous manner, and second electrode-forming face 104a and second electrode side surface 1011c may be formed in a contiguous manner.

In some cases, nanogap NG and channel 107 may be formed along center axis O without deviating from each other. This may make it easier for an object to be measured to pass through channel 107 and nanogap NG along center axis O. Furthermore, first electrode-forming face 103a and first electrode side surface 1010c may be formed in a contiguous manner, and second electrode-forming face 104a and second electrode side surface 1011c may be formed in a contiguous manner. This may minimize differences in level between first electrode-forming face 103a and first electrode side surface 1010c, and can also minimize differences in level between second electrode-forming face 104a and second electrode side surface 1011c. Accordingly, passing of an object to be measured from channel 107 to nanogap NG may be facilitated, and thus an object to be measured, which may flow in channel 107, and may pass more easily through nanogap NG than conventionally.

In some cases, nanogap NG may be formed between first electrode side surface 1010c of first electrode 1010 and second electrode side surface 1011c of second electrode 1011, and channel 107 may also be formed between first electrode-forming face 103a and second electrode-forming face 104a, by removing sidewall spacer 1014 which may be formed between first electrode side surface 1010c of first electrode 1010 and second electrode side surface 1011c of second electrode 1011 and between first electrode-forming face 103a and second electrode-forming face 104a in a contiguous manner during a production process.

As described hereinabove, for nanogap electrode device 101, sidewall spacer 1014 may be removed during a production process, so that channel 107 may be formed by wall-like first electrode-forming face 103a and wall-like second electrode-forming face 104a which may be arranged to face each other while maintaining a constant distance therebetween so as to conform to the shape of sidewall spacer 1014, and so that nanogap NG may be formed at the same time so as to be contiguous between wall-like first electrode side surface 1010c and wall-like second electrode side surface 1011c so as to conform to a shape of sidewall spacer 1014 across center axis O of channel 107 as a center. Accordingly, an object to be measured which may flow in channel 107 may pass along a same center axis O through channel 107 and nanogap NG without deviation between nanogap NG and channel 107, so that the object to be measured which may flow in channel 107 may more easily pass through nanogap NG than conventionally.

In other cases for methods for production of nanogap electrode devices, after sidewall spacer 1014 may be formed in an erect manner between first process layer 1012 and second process layer 1015, first electrode-embedded layer 103 in which first electrode 1010 may be embedded in a surface of first process layer 1012, may be formed so that first electrode 1010 may be brought into contact with a part of sidewall spacer 1014, and second electrode-embedded layer 104 in which second electrode 1011 may be embedded in a surface of second process layer 1015 so that second electrode 1011 may be arranged opposite to first electrode 1010 across sidewall spacer 1014.

In some cases for production methods, wall-like sidewall spacer 1014, which may be sandwiched between first electrode 1010 and second electrode 1011, and which may also be sandwiched between first electrode-embedded layer 103 and second electrode-embedded layer 104, may be removed, so that nanogap NG may be formed at a region at which sidewall spacer 1014 between first electrode 1010 and the second electrode 1011 was removed, and that nanogap NG, which may be in communication with channel 107, may be formed at a region at which sidewall spacer 1014 between first electrode-embedded layer 103 and second electrode-embedded layer 104 was removed.

As described hereinabove, in some cases for production methods, channel 107 may be formed by wall-like first electrode-forming face 103a and wall-like second electrode-forming face 104a, which may be arranged to face each other while maintaining a constant distance therebetween so as to conform to a shape of sidewall spacer 1014, and nanogap NG may be formed contiguously between wall-like first electrode side surface 1010c and wall-like second electrode side surface 1011c to conform to a shape of sidewall spacer 1014 across center axis O of channel 107 as a center. Accordingly, nanogap electrode device 101 may be produced in which an object to be measured, which may flow in channel 107, may pass along a same center axis O through channel 107 and nanogap NG without deviation between nanogap NG and channel 107, so that the object to be measured which may flow in channel 107 may more easily pass through nanogap NG than conventionally.

Surfaces of the first electrode 1010 and second electrode 1011 may be coplanar (or flush) with surfaces of the channel 107. Surfaces of the first electrode 1010 and second electrode 1011 may be continuous with surfaces of the channel 107 that are adjacent to the first electrode 1010 and second electrode 1011. The first electrode side surface 1010c and second electrode side surface 1011c may be coplanar with adjoining surfaces of the channel. In an example, the first electrode-forming face 103a and second electrode-forming face 104a are coplanar with the first electrode side surface 1010c and second electrode side surface 1011c, respectively.

In some examples, the channel 107 has a first width and the first electrode 1010 and second electrode 1011 are spaced apart by a second width, and the first width is substantially the same as the second width. The first width and second width may vary by at most 30 nanometers (nm), 20 nm, 10 nm, 5 nm, 4 nm, 3 nm, 2 nm, or 1 nm from one another.

In other cases for methods for production of nanogap electrode devices, sidewall spacer 1014 may be integrally formed between first electrode-embedded layer 103 and second electrode-embedded layer 104 and between first electrode 1010 and second electrode 1011 in a contiguous manner during a production process. Thus, only by removing sidewall spacer 1014 nanogap NG may be formed between first electrode 1010 and second electrode 1011, and channel 107 may also be formed between first electrode-embedded layer 103 and second electrode-embedded layer 104 at the same time. Therefore, a production process may be simplified compared with a case of independently forming a nanogap and a channel.

In some cases for nanogap electrode devices, by adjusting a film thickness of sidewall spacer 1014, nanogap NG may be formed so as to have a desired width W1 and channel 107 may have a width adjusted to correspond to a width of nanogap NG, and said widths may be formed at the same time. In particular, a film thickness of sidewall spacer 1014 may be made very thin, so that nanogap NG may have a very small width W1 corresponding to a width of sidewall spacer 1014, and channel 107 may be formed so as to have a small width corresponding to a width of nanogap NG. For example, compared with a case of forming a nanogap and a channel by simply etching a surface of first electrode-embedded layer 103, nanogap NG having a smaller width W1 and channel 107 having a smaller width that corresponds to nanogap NG, may be formed.

In some cases for nanogap electrode devices, first electrode-embedded layer 103 and second electrode-embedded layer 104 may be formed of an insulating material. Thus, even when first electrode 1010 may be formed so as to be embedded in a surface of first electrode-embedded layer 103, and second electrode 1011 may be formed so as to be embedded in a surface of second electrode-embedded layer 2, a voltage can be reliably applied only across electrode 1010 and second electrode 1011. Therefore, when a single-stranded DNA molecule may pass through nanogap NG, values of current between first electrode 1010 and second electrode 1011 may be reliably measured.

In some cases for nanogap electrode devices, solution supply part 108 may be provided which may be formed to be wider than a width of channel 107, and which may be in communication with one end of channel 107. Thus, even if channel 107, which may have a very small width may be formed, a supply pump, or other devices for effectuating flow of solutions, may be positioned at wide solution supply part 108, and may easily supply a solution from solution supply part 108 into channel 107.

In some cases for nanogap electrode devices, solution discharge part 109 may be provided which may be formed wider than a width of channel 107, and which may be in communication with another end of channel 107. Thus, even if channel 107 is formed with a very small width, a discharge pump, of other devices for effectuating flow of solutions, may be placed at wide solution discharge part 109, and it may easily discharge a solution from channel 107 to solution discharge part 109. Solution discharge part 109 may temporarily store a solution, so that overflow of solution from channel 107 may be prevented. Furthermore, solution supply part 108 may serve as a solution discharge part, whereas solution discharge part 109 may serve as a solution supply part, accordingly.

In some cases linear channel 107 may have a nanogap NG at a center thereof. Alternatively or additionally, a linear slot in which a nanogap NG may be disposed at a position displaced from a center thereof, and a curved slot in which a nanogap NG may be disposed at a position meeting a center thereof, or at a position displaced from a center thereof, may be used. For example, such a curved slot may be produced by controlling a shape of sidewall spacer 1014 formed during a production process.

In some cases for a method for production of nanogap electrode device 101 as shown in FIG. 17, production of nanogap electrode device 1 may be different from a method for production of the nanogap electrode device 101 as described herein above, in that a lift-off process (described later) may be used for forming a first electrode 1010 and a second electrode 1011. A nanogap electrode device produced by the production method utilizing a lift-off method may have a same configuration as that of nanogap electrode device 101 produced by a production method as described hereinabove, and explanation thereof will be omitted.

In some cases for production methods, utilizing a lift-off method may be substantially similar to a production method as described herein above until a processing step as illustrated in FIG. 20A and FIG. 20B. In other words, as shown in FIG. 20A and FIG. 20B, a first electrode embedment recess 1023a may be formed in a first process layer 1012, and a second electrode embedment recess 1023b, which may be arranged to face first electrode embedment recess 1023a across a sidewall spacer 1014, may be formed in second process layer 1015. Then, wall-like sidewall spacer 1014 may be provided in a manner so as to stand up between first electrode embedment recess 1023a and second electrode embedment recess 1023b.

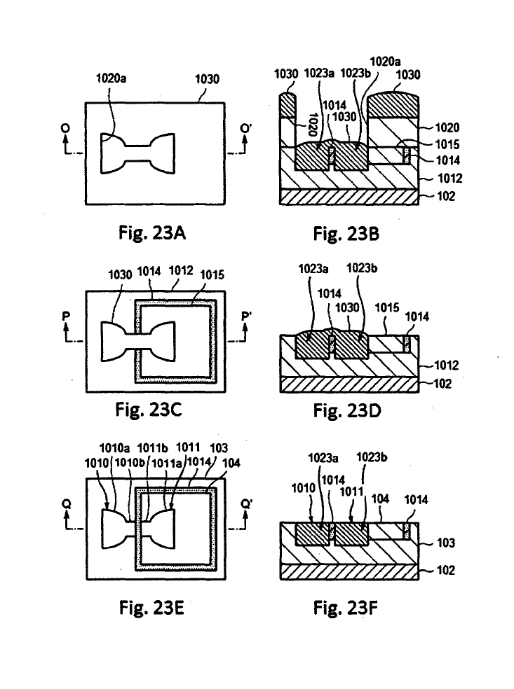

Thereafter, in some cases for methods for production of a nanogap electrode device 101 utilizing a lift-off method as shown in FIG. 23A, in which similar reference numerals are used to denote parts corresponding to those in FIG. 20A, and as shown in FIG. 23B, illustrating a side sectional view taken along the line O-O′ in FIG. 23A, an electrode layer 1030 formed of an insulating material such as gold (Au) may be formed on a surface of an electrode-forming mask 1020, and on surfaces of a first process layer 1012 and a sidewall spacer 1014 which may be exposed through opening part 1020a of electrode-forming mask 1020, for example, using a CVD method, a sputtering method, a plating method, etc. At this time, a configuration, in which each of a first electrode embedment recess 1023a and a second electrode embedment recess 1023b may be filled with an insulating material so that an electrode layer 1030 may be formed therein, may be provided.

After this, by removing electrode-forming mask 1020, electrode layer 1030 on a surface of electrode-forming mask 1020 may be removed together with electrode-forming mask 1020. Thus, electrode layer 1030 may be left within first electrode embedment recess 1023a and within second electrode embedment recess 1023b, as shown in FIG. 23C, in which similar reference numerals are used to denote parts corresponding to those in FIG. 23A, and as shown in FIG. 23D, illustrating a side sectional view taken along the line P-P′ in FIG. 23C. Electrode layer 1030 may remain on a surface of sidewall spacer 1014 that may be provided in an erect manner between first electrode embedment recess 1023a and second electrode embedment recess 1023b. First electrode embedment recess 1023a and second electrode embedment recess 1023b may be formed to have contour shapes of finally formed first electrode 1010 and second electrode 1011 shown in FIG. 17. By making electrode layer 1030 remain within said recesses, electrode layer 1030 having contour shapes of first electrode 1010 and second electrode 1011 may be formed.

Subsequently, as shown in FIG. 23E, in which similar reference numerals are used to denote parts corresponding to those in FIG. 23C, and as shown in FIG. 23F, illustrating a side sectional view taken along the line Q-Q′ in FIG. 23E, a planarizing process may be conducted to polish a surface of electrode layer 1030, and surfaces of sidewall spacer 1014 and second process layer 1015, etc., for example, using a CMP method, until a top face of sidewall spacer 1014 may be exposed, so that first electrode 1010 may be formed in first electrode embedment recess 1023a and second electrode 1011 may be formed in second electrode embedment recess 1023b.

Thus, first electrode-embedded layer 103, in which first electrode 1010 may be embedded in a surface of first electrode embedment recess 1023a, may be formed from first process layer 1012, and second electrode-embedded layer 104, in which second electrode 1011 may be embedded in a surface of second electrode-embedding recess 1023b, may be formed from second process layer 1015. First electrode 1010 and second electrode 1011 that may be formed in this way may be arranged so that side surfaces of nanogap-forming portions 1010b and 1011b thereof face each other across sidewall spacer 1014.

Subsequent processing steps are similar to those of a method of production of a nanogap electrode device 101 as previously described hereinabove. Through the processing steps illustrated in FIGS. 21A to 21F and FIGS. 22A to 22D, a nanogap electrode device 101 as illustrated in FIG. 17 may be produced.

In some cases for a nanogap electrode device 101 produced utilizing a lift-off method utilizing a configuration as described hereinabove, said nanogap electrode device 101 may function in a manner similar to that produced by other methods of production as described hereinabove. In some cases utilizing a lift-off method, sidewall spacer 1014 formed between first electrode side surface 1010c of first electrode 1010 and second electrode side surface 1011c of second electrode 1011 and between first electrode-forming face 103a and second electrode-forming face 104a in a contiguous manner, may be removed. As a result, channel 107 may be formed by wall-like first electrode-forming face 103a and wall-like second electrode-forming face 104a which may be arranged to face each other while maintaining a constant distance therebetween so as to conform to a shape of sidewall spacer 1014, and nanogap NG may be formed at the same time so as to be contiguous between wall-like first electrode side surface 1010c and wall-like second electrode side surface 1011c so as to conform to a shape of sidewall spacer 1014 across center axis O of channel 107 as a center.

In some cases for production of a nanogap electrode devices produced utilizing a lift-off method, an object to be measured which may flow in channel 107 may pass along a same center axis O through channel 107 and nanogap NG without deviation between nanogap NG and channel 107, so that the object to be measured flowing in channel 107 may more easily pass through nanogap NG than conventionally.

In some cases for a production methods utilizing lift-off, after sidewall spacer 1014 may be formed in an erect manner between first process layer 1012 and second process layer 1015, a patterned electrode-forming mask 1020 may be used to form first electrode embedment recess 1023a in first process layer 1012, and second electrode embedment recess 1023b, which may be arranged opposite to first electrode embedment recess 1023a across sidewall spacer 1014, may be formed in second process layer 1015.

In a manner different from a non-lift-off production method, in some cases according to production methods utilizing a lift-off method, electrode layer 1030 may be formed on a surface of electrode-forming mask 1020, and may be formed in first electrode embedment recess 1023a and second electrode embedment recess 1023b may be exposed through opening part 1020a in electrode-forming mask 1020, and subsequently, electrode-forming mask 1020 may be removed. Thus, electrode layer 1030 may be made to remain within first electrode embedment recess 1023a and second electrode embedment recess 1023b by removing electrode layer 1030 on a surface of electrode-forming mask 1020 together with electrode-forming mask 1020. Then electrode layer 1030 bulging out of first electrode embedment recess 1023a and second electrode embedment recess 1023b may be planarized, so that first electrode 1010 is formed in first electrode embedment recess 1023a, and second electrode 1011 may be formed in second electrode embedment recess 1023b.

In some cases for a production method utilizing a lift-off method, a sidewall spacer 1014 may be formed between first electrode side surface 1010c of first electrode 1010 and second electrode side surface 1011c of second electrode 1011 and between first electrode-forming face 103a and second electrode-forming face 104a in a contiguous manner. Subsequent processing steps may be similar to those for a non-lift-off production method as described hereinabove. By removing sidewall spacer 1014 during a production process, channel 107 may be formed between wall-like first electrode-forming face 103a and wall-like second electrode-forming face 104a which may be arranged to face each other while maintaining a constant distance therebetween so as to conform to a shape of sidewall spacer 1014, and nanogap NG may be formed similarly at the same time so as to be contiguous between wall-like first electrode side surface 1010c and wall-like second electrode side surface 1011c to conform to a shape of sidewall spacer 1014 across center axis O of the channel 107 as a center. Thus, using these production methods, a nanogap electrode device 101 may be produced in which an object to be measured can pass along a same center axis O through channel 107 and nanogap NG without deviation between nanogap NG and channel 107, so that said object to be measured flowing in channel 107 may more easily pass through nanogap NG than conventionally.

In some production methods utilizing a lift-off method, by only removing sidewall spacer 1014 that may be formed between first electrode-embedded layer 103 and second electrode-embedded layer 104, nanogap NG may be formed between first electrode 1010 and second electrode 1011 and channel 107 may be formed between first electrode-embedded layer 103 and second electrode-embedded layer 104, at the same time. Thus, a production process may be simplified, compared with the case of independently forming a nanogap and a channel.

Furthermore, in some cases for producing a nanogap electrode device 101 utilizing a lift-off method, nanogap NG having a desired width W1 and channel 107 having a width adjusted to meet nanogap NG may be formed by adjusting a film thickness of sidewall spacer 1014. In particular, a film thickness of sidewall spacer 1014 may be made very thin, so that nanogap NG having a very small width W1 corresponding to a width of sidewall spacer 1014, and channel 107 having a small width corresponding to nanogap NG may be formed. For example, compared with a case of forming a nanogap and a channel by simply etching a surface of first electrode-embedded layer 103, nanogap NG having a smaller width W1 and channel 107 having a smaller width that corresponds to nanogap NG may be formed.

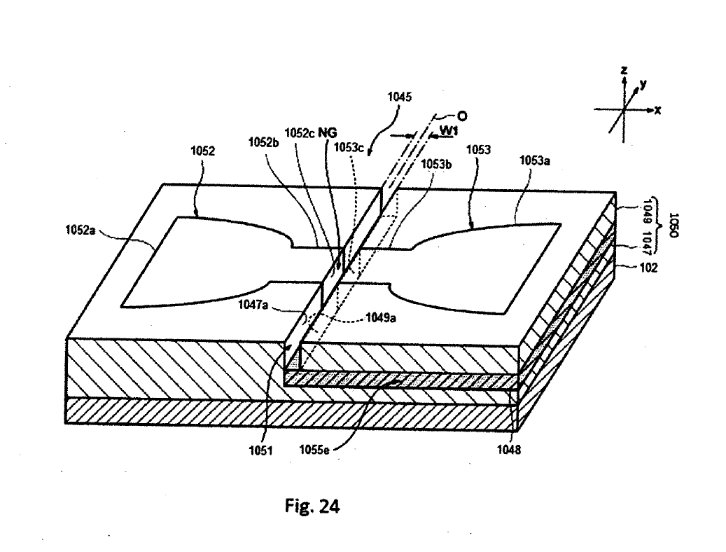

In some cases as shown in FIG. 24, a nanogap electrode device 1045 nanogap may be formed differently in that a lower spacer 1048 may be formed in a shoulder 1055e of a first electrode-embedded layer 1047, and a second electrode-embedded layer 1049 may be formed on lower spacer 1048. In practice, nanogap electrode device 1045 may have a configuration in which an electrode-forming substrate 1050 may comprise first electrode-embedded layer 1047 and second electrode-embedded layer 1049, and electrode-forming substrate 1050 may be disposed on silicon substrate 2.

In electrode-forming substrate 1050, a first electrode 1052, formed of titanium nitride, etc., is embedded in a surface of first electrode-embedded layer 1047, and second electrode 1053, which may be similarly formed of titanium nitride or other similar materials, may be embedded in a surface of second electrode-embedded layer 1049, and slot-like channel 1051 may be formed between first electrode-embedded layer 1047 and second electrode-embedded layer 1049. First electrode 1052 is configured so that band-like nanogap-forming portion 1052b may be integrally formed with a generally semicircular base portion 1052a at a center of an arc thereof, and a wall-like first electrode side surface 1052c of nanogap-forming portion 1052b may be exposed to an inside surface of channel 1051. Second electrode 1053 may be formed to be substantially left-right symmetrical relative to first electrode 1052 with nanogap NG (described later) as a center. Similarly to first electrode 1052, second electrode 1053 may be configured so that band-like nanogap-forming portion 1053b may be integrally formed with a generally semicircular base portion 1053a at a center of the arc thereof, and wall-like second electrode side surface 1053c of nanogap-forming portion 1053b may be exposed to an inside surface of channel 1051.

Nanogap-forming portion 1052b of first electrode 1052 and nanogap-forming portion 1053b of second electrode 1053 may be arranged so that first electrode side surface 1052c and second electrode side surface 1053c may face each other across nanogap NG having a nanoscale width W1. Nanogap NB may be formed to have a width of 2 nm or less, or 1 nm or less, as required according to intended use.

In other cases for electrode-forming substrate 1050, a first electrode-forming face 1047a may be formed in first electrode-embedded layer 1047 and a second electrode-forming face 1049a formed in second electrode-embedded layer 1049 may be arranged to face each other while maintaining a constant distance therebetween, and channel 1051 may be formed between first electrode-forming face 1047a and second electrode-forming face 1049a. Wall-like first electrode side surface 1052c of first electrode 1052 may be exposed on first electrode-forming face 1047a, and wall-like second electrode side surface 1053c of second electrode 1053 may be exposed on second electrode-forming face 1049a, and nanogap NG, which may be in fluid communication with channel 1051, may be formed between first electrode side surface 1052c of first electrode 1052 and second electrode side surface 1053c of second electrode 1053. Furthermore, first electrode side surface 1052c of first electrode 1052 and second electrode side surface 1053c of second electrode 1053 may be arranged to face each other across center axis O of channel 1051 as a center while maintaining a constant distance therebetween.

In some cases, channel 1051 may be linearly formed, and nanogap NG may be disposed at a center thereof. When a solution containing one or more single-stranded DNA molecules that may be an object to be measured may be supplied from one end of channel 1051, said solution may be discharged from another end of channel 1051 through nanogap NG.

First electrode-embedded layer 1047 of electrode device-forming substrate 1050 may be formed of an insulating material such as a silicon oxide, and may be provided on silicon substrate 2. A step may be formed on a surface of a selected region of first electrode-embedded layer 1047, and second electrode-embedded layer 1049 may be provided on a bottom surface in shoulder 1055e via layer-like lower spacer 1048. A part of a side surface of said step part of first electrode-embedded layer 1047 may form first electrode-forming face 1047a which may form channel 1051, and may be formed so that first electrode side surface 1052c of first electrode 1052 may be exposed on first electrode-forming face 1047a.

Second electrode-embedded layer 1049 may comprise second electrode-forming face 1049a which may partly form channel 1051 on a peripheral surface thereof, which may be disposed opposite to first electrode-forming face 1047a of first electrode-embedded layer 1047. Second electrode-embedded layer 1049 may be arranged so that second electrode-forming face 1049a may be disposed to face first electrode-forming face 1047a of first electrode-embedded layer 1047 while maintaining a constant distance therebetween. Second electrode-embedded layer 1049 may comprise second electrode side surface 1053c of second electrode 1053, which second electrode side surface 1053c may be disposed to face with first electrode side surface 1052c of first electrode 1052, and may be exposed on second electrode-forming face 1049a to outside. In this way, in electrode-forming substrate 1050, first electrode-forming face 1047a provided on a side surface of a step of first electrode-embedded layer 1047 and second electrode-forming face 1049a provided on a peripheral surface of second electrode-embedded layer 1049 may be arranged to face each other across center axis O of the channel 1051 as a center while maintaining a constant distance therebetween constant, so that channel 1051 may be formed.

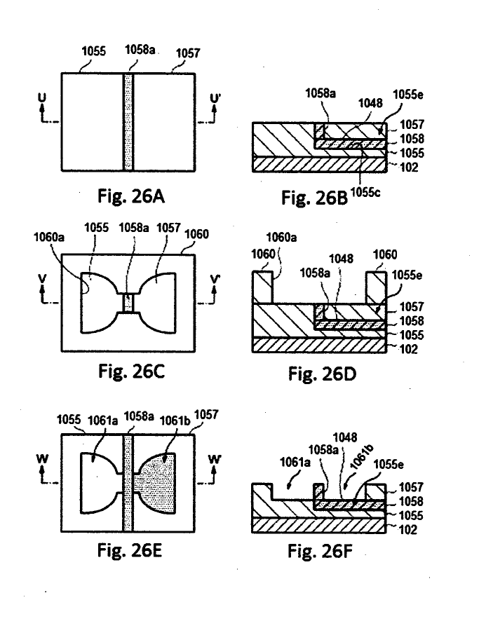

In some cases, lower spacer 1048 may be formed from layers of silicon nitride and other materials as appropriate, which together with a sidewall spacer (described later referring to FIG. 26A and FIG. 26B) may be used during a production process for forming nanogap NG between first electrode 1052 and second electrode 1053 and forming channel 1051 between first electrode-embedded layer 1047 and second electrode-embedded layer 1049. In a processing step for removing said sidewall spacer, lower spacer 1048 may be retained. In some cases, lower spacer 1048 may be exposed through a gap between first electrode-forming face 1047a and second electrode-forming face 1049a, as a bottom surface of channel 1051. Lower spacer 1048, which may be partly exposed as a bottom surface of channel 1051, may be formed in a planar shape over an entirety of channel 1051 and nanogap NG. Thus, a depth of a gap for channel 1051 and a depth of nanogap NG may be made the same.

For such a nanogap electrode device 1045, for example, when a solution containing one or more single-stranded DNA molecules may be supplied to one end of channel 1051 by a supply pump or other device or system for flowing said solution (not shown), said solution containing said one or more single-stranded DNA molecule may be fed to another end of channel 1051 through nanogap NG, and said solution may be discharged from another end of channel 1051 by a discharge pump or other device or system for flowing said solution (not shown). In some cases of nanogap electrode device 1045, solution supply part 108 and solution discharge part 109 as shown in FIG. 17 may not be formed. However, similar to FIG. 17, solution supply part 108 may be provided at one end of channel 1051, and solution discharge part 109 may be provided at another end of channel 1051. In this case, lower spacer 1048 may be exposed in solution supply part 108 and solution discharge part 109.

In some cases utilizing a nanogap electrode device 103, when a voltage may be applied across first electrode 1052 and second electrode 1053 by a power source (not shown), and one or more single-stranded DNA molecules of said solution may flow through nanogap NG between first electrode 1052 and second electrode 1053, values of current flowing through first electrode 1052 and second electrode 1053 may be measured by an ammeter, and bases that comprise said one or more single-stranded DNA molecules may be identified based on current values. At this time, by appropriately selecting a gap width of nanogap NG between first electrode 1052 and second electrode 1053, nanogap electrode device 1045 may analyze a sample with high sensitivity.

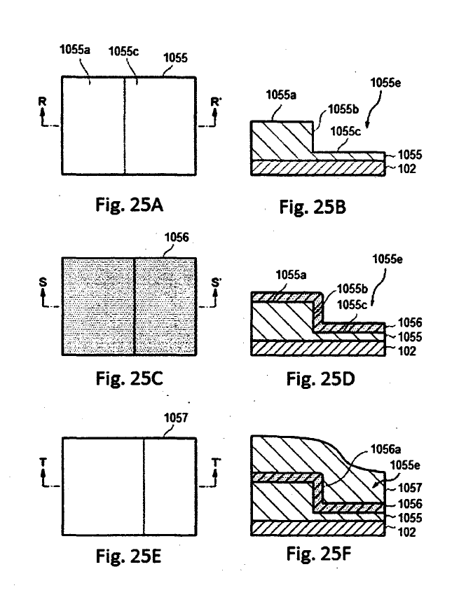

In some cases for methods for production of a nanogap electrode device 1045, first process layer which may be formed of silicon oxide may be deposited on an entire surface of silicon substrate 2, for example, by a CVD method, an ALD method, a sputtering method, a thermal oxidation method, or any other appropriate method or process. Then, first process layer 1055 may be patterned using a photolithographic technique, so that a difference in level may be formed in a surface of first process layer 1055, thereby providing a shallow shoulder 1055e, as shown in FIG. 25A, and as shown in FIG. 25B, illustrating a side sectional view taken along the line R-R′ in FIG. 25A. First process layer 1055 may comprise top surface 1055a of a thick region, bottom surface 1055c in shallow shoulder 1055e, and side surface 1055b with a height corresponding to a depth of shoulder 1055e.

Subsequently, as shown in FIG. 25C, in which similar reference numerals are used to denote parts corresponding to those in FIG. 25A, and as shown in FIG. 25D, illustrating a side sectional view taken along the line S-S′ in FIG. 25C, sidewall spacer-forming layer 1056 formed of an insulating material such as silicon nitride may be deposited on top surface 1055a and on shoulder 1055e of first process layer 1055, for example, using a CVD method, an ALD method, a sputtering method or any other appropriate method or process. As shown in FIG. 25D, sidewall spacer-forming layer 1056 may be deposited on top surface 1055a of first process layer 1055 and on bottom surface 1055c of shoulder 1055e, and may also be deposited on side surface 1054b of shoulder 1055e. At this time, a film thickness of sidewall spacer-forming layer 1056 may be determined depending on a desired width W1 of nanogap NG or width of channel 1051 that may be formed to meet nanogap NG. In other words, when nanogap NG with a small width W1, and channel 1051 with a small width so as to be consistent therewith, may be formed, a film thickness of sidewall spacer-forming layer 1056 may be made so as to be small, whereas when nanogap NG with a large width W1, and channel 1051 with a large width, so as to be consistent therewith, may be formed, a film thickness of sidewall spacer-forming layer 1056 may be made so as to be large.

In some cases as shown in FIG. 25E, in which similar reference numerals are used to denote parts corresponding to those in FIG. 25C, and as shown in FIG. 25F, illustrating a side sectional view taken along the line T-T′ in FIG. 25E, second process layer 1057, which may be formed of silicon oxide or other appropriate materials, may be formed on sidewall spacer-forming layer 1056, for example, using a CVD method, an ALD method, a sputtering method or any other appropriate method or process. At this time, second process layer 1057 may be formed to also overlie side surface 1056a of sidewall spacer-forming layer 1056 that may be formed alongside surface 1055b of first process layer 1055.

Subsequently as shown in FIG. 26A, in which similar reference numerals are used to denote parts corresponding to those in FIG. 25E, and as shown in FIG. 26B, illustrating a side sectional view taken along the line U-U′ in FIG. 26A, a surface of second process layer 1057 and a surface of sidewall spacer-forming layer 1056 may be subjected to planarization, for example, using a CMP (Chemical Mechanical Polishing) method or any other appropriate method or process, so that a surface of first process layer 1055 and a surface of second process layer 1057 may be exposed.

As a result, sidewall spacer-forming layer 1056 may be removed at a region formed on a surface of first process layer 1055, so that a spacer layer 1058 of L-shaped cross section remains, which may be constituted of a wall-like sidewall spacer 1058a provided in an erect manner between first process layer 1055 and second process layer 1057, and layer-like lower spacer 1048 which may be integrally formed with sidewall spacer 1058a at a lower end thereof, and may extend between bottom surface 1055c of shoulder 1055e and second process layer 1057.

Next as shown in FIG. 26C, in which similar reference numerals are used to denote parts corresponding to those in FIG. 26A, and as shown in FIG. 26D, illustrating a side sectional view taken along the line V-V in FIG. 26C, a photoresist may be applied to a surface of first process layer 1055, a surface of sidewall spacer 1058a, and a surface of second process layer 1057 to form a photoresist layer. Then, said photoresist layer may be patterned using a photolithographic technique. Thus, an electrode-forming mask 1060 may be formed, in which opening parts 1060a, each corresponding to each contour shape of first electrode 1052 and second electrode 1053 (FIG. 24), may be patterned. Through opening part 1060a patterned in electrode-forming mask 1060, first process layer 1055 and second process layer 1057, between which sidewall spacer 1058a may be sandwiched, may be exposed.

Subsequently as shown in FIG. 26E, in which similar reference numerals are used to denote parts corresponding to those in FIG. 26C, and as shown in FIG. 26F illustrating a side sectional view taken along the line W-W′ in FIG. 26E, surfaces of first process layer 1055 and second process layer 1057 may be exposed through opening parts 1060a of electrode-forming mask 1060 may be etched, for example, by dry etching. After etching, electrode-forming mask 1060 may be removed. By this processing step, first electrode embedment recess 1061a having a same contour shape as that of first electrode 1052 may be formed in first process layer 1055, and sidewall spacer 1058a having a same contour shape as that of second electrode 1053, which may be arranged to face first electrode embedment recess 1061a across a second electrode embedment recess 1061b, may be formed in second process layer 1057.

Thus, sidewall spacer 1058a may remain between first electrode embedment recess 1061a and second electrode embedment recess 1061b, and wall-like sidewall spacer 1058a may be provided in an erect manner between first electrode embedment recess 1061a and second electrode embedment recess 1061b. In some cases first process layer 1055 and second process layer 1057 may be etched and lower spacer may be exposed in second electrode embedment recess 1061b. Alternatively or additionally, first process layer 1055 and second process layer 1057 may be etched so that only a part of each side surface of sidewall spacer 1058a may be exposed, so that second process layer 1057 may remain in second electrode embedment recess 1061b without exposing lower spacer 1048.

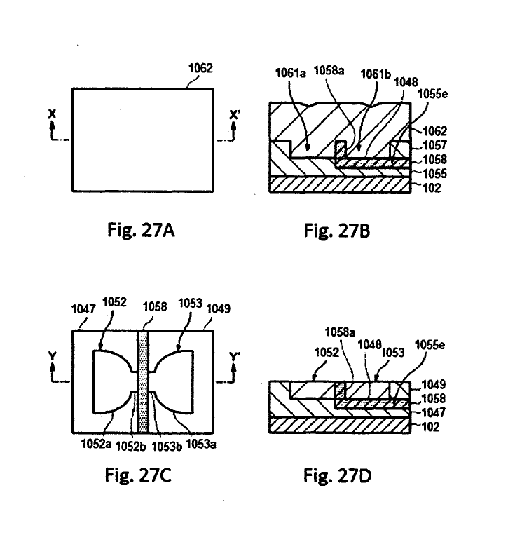

Thereafter, as shown in FIG. 27A, in which similar reference numerals may be used to denote parts corresponding to those in FIG. 26E, and as shown in FIG. 27B, illustrating a side sectional view taken along the line X-X′ in FIG. 27A, electrode layer 1062, which may be formed of titanium nitride or other appropriate materials, may be formed on a surface of first process layer 1055, an exposed surface of spacer layer 1058, and a surface of second process layer 1057, for example, using a CVD method. At this time, as shown in FIG. 27B, first electrode embedment recess 1061a and second electrode embedment recess 1061b may be filled with titanium nitride, and thus electrode layer 1062 may be formed.

Subsequently as shown in FIG. 27C, in which similar reference numerals are used to denote parts corresponding to those in FIG. 27A, and as shown in FIG. 27D illustrating a side sectional view taken along the line Y-Y′ in FIG. 27C, a planarizing process may be conducted on a surface of electrode layer 1062 until an upper end of sidewall spacer 1058a may be exposed, for example, using a CMP method. Thus, first electrode 1052 may be formed in first electrode embedment recess 1061a, and second electrode 1053 may be formed in second electrode embedment recess 1061b.

Thus, first electrode-embedded layer 1047, in which first electrode 1052 may be embedded in a surface of first electrode embedment recess 1061a, may be formed from first process layer 1055, and second electrode-embedded layer 1049, in which second electrode 1053 is embedded in a surface of second electrode embedment recess 1061b, may be formed from second process layer 1057. First electrode 1052 and second electrode 1053 may be formed in this way such that side surfaces of nanogap-forming portions 1052b and 1053b thereof may be arranged to face each other across sidewall spacer 1058a.

Thereafter, sidewall spacer 1058a, a top face of which may be exposed and subsequently removed as a result of removal of portions of spacer layer 1058, for example, by dry etching, so that portions of spacer layer 1058 remains as a result of being covered by second electrode-embedded layer 1049 and second electrode 1053. As a result, nanogap NG may be formed between first electrode side surface 1052c of first electrode 1052 and second electrode side surface 1053c of second electrode 1053 at a region from which sidewall spacer 1058a may be removed. Furthermore, channel 1051 having a same width as that of nanogap NG may be formed between first electrode-embedded layer 1047 and second electrode-embedded layer 1049 at a region from which sidewall spacer 1058a may be removed. Through processing steps described hereinabove and as shown in FIG. 24, channel 1051 may be formed such that first electrode-forming face 1047a of first electrode-embedded layer 1047 and second electrode-forming face 1049a of second electrode-embedded layer 1049 may be arranged to face each other across center axis O as a center while maintaining a constant distance therebetween. In some cases nanogap electrode device 1045 may be formed such that first electrode side surface 1052c of first electrode 1052 and second electrode side surface 1053c of second electrode 1053 may be arranged to face each other across center axis O as a center while maintaining a constant distance therebetween.

In some cases, sidewall spacer 1058a may be completely removed so that lower spacer 1048 may remain in channel 1051 as shown in FIG. 24. Alternatively or additionally, not only sidewall spacer 1058a, but also an entirety of lower spacer 1048 exposed in channel 1051 may be removed so as to expose first electrode-embedded layer 1047 in channel 1051. In some cases, at a region below nanogap NG, first electrode 1052 may not face with second electrode 1053, and a region, at which first electrode-embedded layer 1047 may face with lower spacer 1048, may be formed. In some cases for such a nanogap electrode device, when one or more single-stranded DNA molecules in a solution flow through a gap between first electrode-embedded layer 1047 and lower spacer 1048, as a result of an electric field that may be generated by first electrode 1052 and second electrode 1053, values of current flowing through first electrode 1052 and second electrode 1053 may change. Based on current value changes, bases that comprise said one or more single-stranded DNA molecules may be identified.

In some cases as described hereinabove, nanogap electrode device 1045 may be provided with first electrode-embedded layer 1047, which may be formed of an insulating material and may comprise first electrode-forming face 1047a, and second electrode-embedded layer 1049, which may also be formed of an insulating material and may comprise second electrode-forming face 1049a. Furthermore, nanogap electrode device 1045 may also be provided with first electrode 1052 comprising first electrode side surface 1052c that may be exposed in first electrode-forming face 1047a, and second electrode 1053 comprising second electrode side surface 1053c that may be exposed in second electrode-forming face 1049a. Furthermore, nanogap electrode device 1045 may be provided with channel 1051 and nanogap NG that may be in fluid communication with channel 1051. Channel 1051 may be defined by first electrode-forming face 1047a and second electrode-forming face 1049a, which may be arranged to face each other while maintaining a constant distance therebetween, and channel 1051 may extends along center axis O between first electrode-forming face 1047a and second electrode-forming face 1049a.

Nanogap NG may be formed between first electrode side surface 1052c and second electrode side surface 1053c, which may be arranged to face each other across center axis O of channel 1051 as a center while maintaining a constant distance therebetween. In some cases for nanogap electrode devices 1045, first electrode-forming face 1047a and first electrode side surface 1052c may be formed in a contiguous manner, and second electrode-forming face 1049a and second electrode side surface 1053c may be formed in a contiguous manner.

In some cases for the nanogap electrode devices as described hereinabove, nanogap NG and channel 1051 may be formed along center axis O without deviating from each other. This may make it easier for an object to be measured to pass through channel 1051 and nanogap NG along center axis O. Furthermore, first electrode-forming face 1047a and first electrode side surface 1052c may be formed in a contiguous manner, and second electrode-forming face 1049a and second electrode side surface 1053c may be formed in a contiguous manner. This may minimize differences in level between first electrode-forming face 1047a and first electrode side surface 1052c, and may also minimize differences in level between second electrode-forming face 1042a and second electrode side surface 1053c. Accordingly, passing of an object to be measured from channel 1051 to nanogap NG may be facilitated, and thus an object to be measured flowing in channel 1051 may more easily pass through nanogap NG than may be possible conventionally.

In some cases for nanogap electrode devices, nanogap NG may be formed between first electrode side surface 1052c of first electrode 1052 and second electrode side surface 1053c of second electrode 1053, and at this time, channel 1051 may also be formed between first electrode-forming face 1047a and second electrode-forming face 1049a, by removing sidewall spacer 1058a which may have been formed both between first electrode side surface 1052c of first electrode 1052 and second electrode side surface 1053c of second electrode 1053 and between first electrode-forming face 1047a and second electrode-forming face 1049a in a contiguous manner during a production process.

In some cases as described hereinabove for nanogap electrode devices, sidewall spacer 1058a may be removed during a production process, so that channel 1051 may be formed between wall-like first electrode-forming face 1047a and wall-like second electrode-forming face 1049a which may be arranged to face each other while maintaining a constant distance therebetween so as to conform to a shape of sidewall spacer 1058a, and so that nanogap NG may be formed at the same time so as to be contiguous between wall-like first electrode side surface 1052c and wall-like second electrode side surface 1053c to conform to a shape of sidewall spacer 1058a along center axis O of channel 1051 as a center. Accordingly, an object to be measured which may flow in channel 1051 may pass through channel 1051 and nanogap NG along center axis O without deviation between nanogap NG and channel 1051, so that an object to be measured flowing in channel 1051 may easily pass through nanogap NG.

In some cases for methods for production of nanogap electrode devices, after sidewall spacer 1058a may be formed in an erect manner between first process layer 1055 and second process layer 1057, first electrode-embedded layer 1047 in which first electrode 1052 may be embedded in a surface of first process layer 1055 may be formed so that first electrode 1052 may be brought into contact with a part of sidewall spacer 1058a, and second electrode-embedded layer 1049 in which second electrode 1053 may be embedded in a surface of second process layer 1057 so that second electrode 1053 may be arranged opposite to first electrode 1052 across sidewall spacer 1058a.

In additional cases for methods of production of nanogap electrode devices, wall-like sidewall spacer 1058a, which may be formed between first electrode 1052 and second electrode 1053, and between first electrode-embedded layer 1047 and second electrode-embedded layer 1049 in a contiguous manner, may be removed, so that nanogap NG conforming to a shape of sidewall spacer 1058a may be formed, and channel 1051 conforming to a shape of sidewall spacer 1058a may be formed between first electrode-embedded layer 1047 and second electrode-embedded layer 1049.

In some cases for methods of production of nanogap electrode devices, channel 1051 may be formed between wall-like first electrode-forming face 1047a and wall-like second electrode-forming face 1049a which may be arranged to face each other while maintaining a constant distance therebetween so as to conform to a shape of sidewall spacer 1058a, and nanogap NG may be formed so as to be contiguous between wall-like first electrode side surface 1052c and wall-like second electrode side surface 1053c so as to conform to a shape of sidewall spacer 1058a along center axis O of channel 1051 as a center. Accordingly, nanogap electrode device 1045 may be produced in which objects to be measured which may flow in channel 1051 may pass through channel 1051 and nanogap NG along center axis O without deviation between nanogap NG and channel 1051, so that an object to be measured flowing in channel 1051 may easily pass through nanogap NG.

In some cases for methods of production of nanogap electrode devices, by removing sidewall spacer 1058a that may have been formed between first electrode-embedded layer 1047 and second electrode-embedded layer 1049, nanogap NG may be formed between first electrode 1052 and second electrode 1053, and channel 1051 may be formed between first electrode-embedded layer 1047 and second electrode-embedded layer 1049, at the same time. Thus, a production process may be simplified, compared with the case of independently forming a nanogap and a channel.

In some cases for a production method, after sidewall spacer-forming layer 1056 that overlies the side surface 1055b may be formed on first process layer 1055 having side surface 1055b in a surface thereof, second process layer 1057 may be formed on sidewall spacer-forming layer 1056, and a planarizing process may be conducted to expose a surface of first process layer 1055 and a surface of second process layer 1057. As a result in some cases, sidewall spacer 1058a that may be provided in an erect manner between first process layer 1055 and second process layer 1057 may be formed. Thus, a step of etching back sidewall spacer 1014 from sidewall spacer-forming layer 1013 as described for some cases hereinabove may be made unnecessary. Thus, a production process can be simplified accordingly.

In some cases for methods of producing nanogap electrode devices, by adjusting film thickness of sidewall spacer 1058a, nanogap NG, having a desired width W1 and channel 1051 having a width adjusted to correspond to nanogap NG, may be formed. In particular, a film thickness of sidewall spacer 1014 may be made very thin, so that nanogap NG having a very small width W1 corresponding to the width of sidewall spacer 1014, and channel 1051 having a small width corresponding to the nanogap NG may be formed. For example, compared with a case of forming a nanogap and a channel by simply etching a surface of first electrode-embedded layer 1047, nanogap NG having a smaller width W1 and channel 1051 having a smaller width that corresponds to the nanogap NG, may be formed.

For some cases for methods of production of nanogap electrode devices, first electrode-embedded layer 1047 and second electrode-embedded layer 1049 may be formed of an insulating material. First electrode 1052 may be formed so as to be embedded in a surface of first electrode-embedded layer 1047, and second electrode 1053 may be formed so as to be embedded in a surface of second electrode-embedded layer 2, and a voltage may be reliably applied only across first electrode 1052 and second electrode 1053. Therefore, when one or more single-stranded DNA molecules pass through nanogap NG, values of current between first electrode 1052 and second electrode 1053 may be reliably measured.

In some cases, a composite nanogap electrode device in which at least two nanogap electrode devices are linked together can be produced. In some cases, multiple nanogap electrode devices may be produced such that that said nanogap electrode devices may share a common channel. In other cases, said nanogap electrode devices may have individual different channels. Is some cases, said nanogap electrode devices may be configured so that several nanogap electrode devices may share each of several different channels.

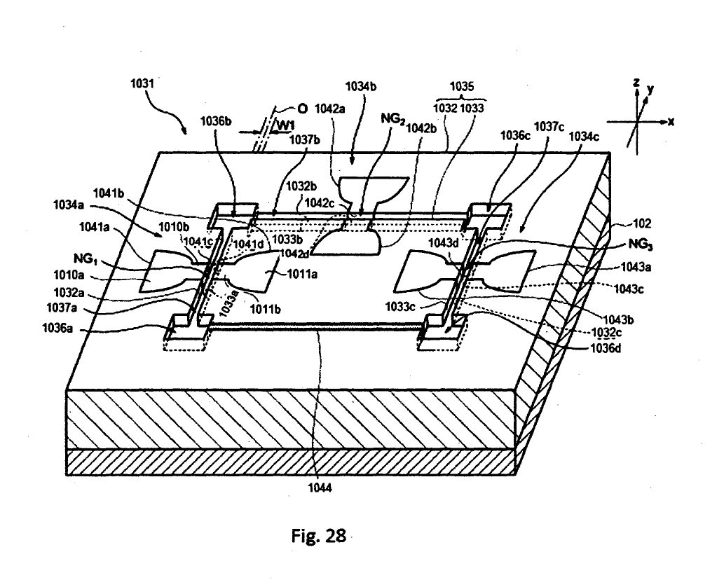

In some cases as illustrated in FIG. 28, a composite nanogap electrode device 1031, which may include at least two nanogap electrode devices 1034a, 1034b, 1034c linked together, may be produced utilizing any of the methods of production as described hereinabove. A composite nanogap electrode device 1031 may be provided by producing at least two nanogap electrode devices at the same time utilizing any of the methods for production of a nanogap electrode device as described hereinabove. Processing steps therefor may correspond to those of cases as shown in FIG. 18A to FIG. 18F.

In some cases for forming composite nanogap electrode device 1031, electrode device-forming substrate 1035 formed of an insulating material such as a silicon oxide or other materials, may be formed on silicon substrate 2, and at least two nanogap electrode devices 1034a, 1034b, 1034c may be produced on electrode device-forming substrate 1035. In some cases, solution supply part 1036a may be recessed, for example, in rectangularly-shaped solution-passing parts 1036b and 1036c, and solution discharge part 1036d, may be formed on electrode device-forming substrate 1035. Furthermore, a slot, from which channel 1037a associated with nanogap electrode device 1034a may be formed, may be provided between solution supply part 1036a and solution-passing part 1036b, a slot, from which channel 1037b associated with nanogap electrode device 1034b may be formed, may be provided between solution-passing part 1036b and a solution-passing part 1036c, and a slot from which a channel 1037c associated with nanogap electrode device 1034c may be formed, may be provided between solution-passing part 1036c and solution discharge part 1036d, may be provided thereon.

In some cases, channel 1037a associated with nanogap electrode device 1034a may be in fluid communication with solution supply part 1036a at one end, and may be in fluid communication with the solution-passing part 1036b at another end, and channel 1037b associated with nanogap electrode device 1034b may be in fluid communication with solution-passing part 1036b at one end, and may be in fluid communication with solution-passing part 1036c at another end. In addition, channel 1037c associated with nanogap electrode device 1034c may be in fluid communication with solution-passing part 1036c at one end and may be in fluid communication with solution discharge part 1036d at another end. Accordingly, a solution supplied to solution supply part 1036a by a supply pump or other appropriate device for causing a solution to flow (not shown) may supply said solution feed through channel 1037a to and through nanogap electrode device 1034a to solution discharge part 1036d through solution-passing part 1036b, channel 1037b associated with nanogap electrode device 1034b, solution-passing part 1036c, and channel 1037c associated with nanogap electrode device 1034c, sequentially, and may be discharged from solution discharge part 1036d by a discharge pump or other device, or may be discharged from solution discharge part by said supply pump (not shown).

As a result, composite nanogap electrode device 1031 may be configured so that a solution may pass through respective nanogaps NG1, NG2, and NG3 (described later) in an order of nanogap electrode devices 1034a, 1034b, and 1034c.

Plurality of nanogap electrode devices 1034a, 1034b, and 1034c may be provided in composite nanogap electrode device 1031, and may have a same structure as that of nanogap electrode device 101 described hereinabove, or may be configured in other manners as described herein. To avoid duplicate description in the following, a description is made focusing on one nanogap electrode device. Thus, nanogap electrode 1034a, among a plurality of nanogap electrode devices 1034a, 1034b, and 1034c, are focused on below. In practice, nanogap electrode device 1034a may be provided with first electrode 1041a formed of titanium nitride, or other appropriate materials, and second electrode 1041b, which may be similarly formed of titanium nitride or other appropriate materials, and nanogap NG1 having a nanoscale width W1 may be provided between first electrode 1041a and second electrode 1041b. First electrode 1041a may be embedded in a surface of electrode device-forming substrate 1035, and band-like nanogap-forming portion 1010b may be integrally formed with a generally semicircular-shaped base portion 1010a at a center of an arc thereof, and wall-like flat first electrode side surface 1041c that may be formed at a tip of nanogap forming part 1010b may be exposed contiguously with an inner surface of channel 1037.

Second electrode 1041b may be formed so as to be substantially left-right symmetrical with respect to first electrode 1041a with nanogap NG1 as a center. Similarly to first electrode 1041a, second electrode 1041b may be formed so as to be embedded in a surface of electrode device-forming substrate 1035. In this case, second electrode 1041b may be configured so that a band-like nanogap-forming portion 1011b may be integrally formed with a generally semicircular base portion 1011a at a center of an arc thereof, and wall-like second electrode side surface 1041d of nanogap-forming portion 1011b may be exposed contiguously with an inner surface of channel 1037a. First electrode side surface 1041c and second electrode 1041b of first electrode 1041a may be arranged to face each other, and nanogap NG1 may be formed between first electrode side surface 1041c and second electrode side surface 1041d.

Nanogap electrode device 1034b may also be provided with first electrode 1042a on which first electrode side surface 1042c may be formed, and second electrode 1042b on which second electrode side surface 1042d may be formed. Furthermore, nanogap NG2 having a nanoscale width W1 may be formed between first electrode side surface 1042c and second electrode side surface 1042d. Nanogap electrode device 1034c may also be provided with first electrode 1043a on which first electrode side surface 1043c may be formed, and second electrode 1043b on which second electrode side surface 1043d may be formed. Furthermore, nanogap NG3 having a nanoscale width W1 may be formed between first electrode side surface 1043c and second electrode side surface 1043d. For some cases of composite nanogap electrode devices, nanogaps NG1, NG2, and NG3, associated with nanogap electrode devices 1034a, 1034b, and 1034c, may all be formed based on a same sidewall spacer(described later). Thus, they may have a same width W1. Width W1 May be formed, for example, to be 10 nm or less, 2 nm or less, or 1 nm or less, as required according to intended use.

In other cases, channel 1037a which may be formed so as to be associated with nanogap electrode device 1034a may be provided between first electrode-forming face 1032a and second electrode-forming face 1033a. First electrode-forming face 1032a and second electrode-forming face 1033a may be arranged to face each other across center axis O as a center while maintaining a constant distance therebetween. First electrode side surface 1041c of first electrode 1041a may be exposed through first electrode-forming face 1032a, whereas second electrode side surface 1041d of second electrode 1041b may be exposed through second electrode-forming face 1033a. First electrode side surface 1041c of first electrode 1041a and second electrode side surface 1041d of second electrode 1041b may be arranged to face each other across a center axis of channel 1037a as a center while maintaining a constant distance therebetween. Nanogap NG1, which may be formed between first electrode side surface 1041c and second electrode side surface 1041d, may be in fluid communication with channel 1037a.

In other cases, nanogap electrode device 1034b, similarly to nanogap electrode device 1034a, channel 1037b may be formed between first electrode-forming face 1032b and second electrode-forming face 1033b. Furthermore, first electrode-forming face 1032b and second electrode-forming face 1033b may be arranged to face each other across a center axis while maintaining a constant distance therebetween. First electrode side surface 1042c of first electrode 1042a may be exposed through first electrode-forming face 1032a, whereas second electrode side surface 1042d of second electrode 1042b may be exposed through second electrode-forming face 1033b. First electrode side surface 1042c of first electrode 1042a and second electrode side surface 1042d of second electrode 1042b may be arranged to face each other across a center axis of channel 1037b while maintaining a constant distance therebetween. Nanogap NG2, formed between first electrode side surface 1042c and second electrode side surface 1042d, may be in fluid communication with channel 1037b.

In some cases, nanogap electrode device 1034c, similarly to nanogap electrode devices 1034a and 1034b, channel 1037c may be formed between first electrode-forming face 1032c and second electrode-forming face 1033c. Furthermore, first electrode-forming face 1032c and second electrode-forming face 1033c may be arranged to face each other across a center axis while maintaining a constant distance therebetween. First electrode side surface 1043c of first electrode 1043a may be exposed through first electrode-forming face 1032c, whereas second electrode side surface 1043d of second electrode 1043b may be exposed through second electrode-forming face 1033c. First electrode side surface 1043c of first electrode 1043a and second electrode side surface 1043d of second electrode 1043b may be arranged to face each other across a center axis of channel 1037c while maintaining a constant distance therebetween. Nanogap NG3, which may be formed between first electrode side surface 1043c and second electrode side surface 1043d, may be in fluid communication with channel 1037c.

In some cases, channel 1037a, 1037b, 1037c may be linearly formed, and nanogap NG1, NG2, NG3 may be disposed at a center thereof. When a solution, which may contain an object to be measured, may be supplied from one end, said solution may be discharged from another end through nanogaps NG1, NG2, NG3. In other words, channels 1037a, 1037b, 1037c may be made to supply a solution to nanogap NG1, NG2, NG3, and to discharge said solution from nanogaps NG1, NG2, NG3.

In some cases, an example is given in which linear channels 1037a, 1037b, and 1037c having nanogap NG at a center thereof may be used. Alternatively or additionally, a linear slot in which a nanogap NG1, NG2, NG3 may be disposed at a position displaced from a center thereof, and a curved slot in which a nanogap NG1, NG2, NG3 may be disposed at a position meeting a center thereof, or at a position displaced from a center thereof, may be used.

In some cases, band-like sidewall spacer 1044 which may be formed of silicon nitride or other suitable materials, may be embedded in a surface of electrode device-forming substrate 1035 between solution supply part 1036a and solution discharge part 1036d. Sidewall spacer 1044 which may have been a part of a sidewall spacer which may have been used for forming nanogaps NG1, NG2, and NG3 of nanogap electrode devices 1034a, 1034b, and 1034c, and may have been used to form channels 1037a, 1037b, and 1037c, during a production process, and which may not have been removed during a production process and may thereby remain. In this case, sidewall spacer 1044 may be connected to solution supply part 1036a at one end, and may be connected to solution discharge part 1036d at another end, and end faces of sidewall spacer 1044 may be exposed at a side surface of solution supply part 1036a and at a side surface of solution discharge part 1036d, respectively.

In some cases for methods for production of composite nanogap electrode device 1031, after sidewall spacer 1044, for example having a quadrilateral shape, may be formed so that sidewall spacer 1044 may be embedded in electrode device-forming substrate 1035 with a surface of sidewall spacer exposed thereat (see FIG. 19C and FIG. 19D), first electrode 1041a (1042a, 1043a) and second electrode 1041b (1042b, 1043b), which may be arranged to face each other across sidewall spacer, may be formed on each of three sides of sidewall spacer 1044. Then, sidewall spacer 1044 between first electrode 1041a (1042a, 1043a) and second electrode 1041b (1042b, 1043b) may be removed, whereby nanogap(s) NG1 (NG2, NG3) may be formed between first electrode(s) 1041a (1042a, 1043a) and second electrode(s) 1041b (1042b, 1043b). Among volumes formed as a result of removing sidewall spacer 1044, volumes other than those for nanogaps NG1, NG2, NG3 may be formed to be channels 1037a, 1037b, 1037c. In FIG. 28, sidewall spacer 1044 that was not removed during a production process, and remains along one side is illustrated.

In some cases, electrode device-forming substrate 1035 on which nanogap electrode devices 1034a, 1034b, 1034c may be formed may be provided with plate-like first electrode-embedded layer 1032, and second electrode-embedded layer 1033 that may be embedded in a recess (not shown) formed in a surface of first electrode-embedded layer 1032. First electrode-embedded layer 1032 may be formed of an insulating material such as silicon oxide, and may be formed on silicon substrate 102. First electrode-embedded layer 1032 may have a recessed region in a surface thereof. In said recessed region, first electrode-forming face 1032a whereby channel 1037a may be formed, first electrode-forming face 1032b whereby channel 1037b may be formed, and first electrode-forming face 1032c whereby channel 1037c, may be formed. Furthermore, in a surface of first electrode-embedded layer 1032, first electrode 1041a of nanogap electrode device 1034a, first electrode 1042a of nanogap electrode device 1034b, and first electrode 1043a of nanogap electrode device 1034c, may be embedded. Furthermore, in first electrode-embedded layer 1032, first electrode side surface 1041c of first electrode 1041a may be exposed at first electrode-forming face 1032a in a manner so as to be flush therewith, first electrode side surface 1042c of first electrode 1042a may be exposed at first electrode-forming face 1032b in a manner so as to be flush therewith, and first electrode side surface 1043c of first electrode 1043a may be exposed at the first electrode-forming face 1032c in a manner so as to be flush therewith.

Second electrode-embedded layer 1033 may be formed of an insulating material such as a silicon oxide, and second electrode-forming faces 1033a, 1033b, and 1033c which may be used for forming channels 1037a, 1037b, and 1037c, and may be formed on a peripheral surface thereof. In a recess formed in a surface of first electrode-embedded layer 1032, second electrode-embedded layer 1033 may be disposed so as to arrange second electrode-forming face 1033a to face first electrode-embedded layer 1032 while maintaining a constant distance therebetween. Second electrode-forming face 1033b may be arranged to face first electrode-forming face 1032b while maintaining a constant distance therebetween, and second electrode-forming face 1033c may be arranged to face first electrode-forming face 1032c while maintaining a constant distance therebetween. Furthermore, sidewall spacer 1044 may be formed along one side of second electrode-embedded layer 1033, which one side may be a side other than three other sides thereof, e.g., second electrode-forming faces 1033a, 1033b, and 1033c.

In some cases, wherein channel 1037a may be formed between first electrode-forming face 1032a and second electrode-forming face 1033a, channel 1037b may be similarly formed between first electrode-forming face 1032b and second electrode-forming face 1033b, and channel 1037c may be similarly formed between first electrode-forming face 1032c and second electrode-forming face 1037c. For some cases for composite nanogap electrode device(s) 1031 having such a configuration, when a solution containing one or more single-stranded DNA molecules may be supplied to solution supply part 1036a, for example, by a supply pump or other device or system for flowing said solution (not shown), said solution may be flowed to solution-passing part 1036b through channel 1037a. In said composite nanogap electrode device(s) 1031, when said solution containing one or more single-stranded DNA molecules passes through channel 1037a, said solution may pass through nanogap NG1 between first electrode 1041a and second electrode 1041b of nanogap electrode device 1034a.

In some cases for nanogap electrode device(s) 1034a, a voltage may be applied between first electrode 1041a and second electrode 1041b by a power source (not shown), and when one or more single-stranded DNA molecules in a solution flow passes through nanogap NG1 between first electrode 1041a and second electrode 1041b, bases that comprise said one or more single-stranded DNA molecules may be identified based on values of current flowing through first electrode 1041a and second electrode 1041b.

In some cases, composite nanogap electrode device 1031 may be configured to supply a solution containing said one or more single-stranded DNA molecules from the solution-passing part 1036b to the solution-passing part 1036c through the channel 1037b after being subjected to base sequence analysis, so that said solution containing said one or more single-stranded DNA molecules may pass through nanogap NG2 between first electrode 1042a and second electrode 1042b provided in channel 1037b.

In some cases for nanogap electrode devices, a voltage may be applied between first electrode 1042a and second electrode 1042b by a power source (not shown), and when said one or more single-stranded DNA molecules in said solution flow through nanogap NG2 between first electrode 1042a and second electrode 1042b, bases that comprise said one or more single-stranded DNA molecules may be identified based on values of current flowing through first electrode 1042a and second electrode 1042b.

In some cases for composite nanogap electrode devices, said solution containing one or more single-stranded DNA molecules may be flowed from solution-passing part 1036c to solution discharge part 1036d through channel 1037c through nanogap NG3 between first electrode 1043a and second electrode 1043b through channel 1037c, and said solution may be discharged from solution discharge part 1036d by a discharge pump or other device or system for flowing said solution, after being subjected to base sequence analysis.

In some cases for nanogap electrode devices, a voltage may be applied across first electrode 1043a and second electrode 1043b by a power source (not shown), and when said one or more single-stranded DNA molecules in a solution flows through nanogap NG3 between first electrode 1043a and second electrode 1043b, bases that constitute single-stranded DNA molecule may be identified based on values of current flowing through first electrode 1043a and second electrode 1043b. Thus, composite nanogap electrode device 1031 may be configured to repeatedly perform base sequence analysis of a same one or more single-stranded DNA molecules, by subjecting said one or more single-stranded DNA molecules to base sequence analysis at each nanogap electrode device 1034a, 1034b, 1034c, sequentially.

For some cases for composite nanogap electrode devices, in which nanogap NG1 may be formed between first electrode side surface 1041c and second electrode side surface 1041d with center axis O of channel 1037a as a center; nanogap electrode device 1034b, in which nanogap NG2 may be formed between first electrode side surface 1042c and second electrode side surface 1042d with center axis O of channel 1037b as a center, and nanogap electrode device 1034c, in which nanogap NG3 may be formed between first electrode side surface 1043c and second electrode side surface 1043d with center axis O of channel 1037c as a center, so that adjacent nanogap electrode devices 1034a, 1034b (1034b, 1034c) may be made so as to be in fluidic communication with each other through channels 1037a, 1037b (1037b, 1037c).

In some cases for composite nanogap electrode devices, similarly to some cases for nanogap electrode devices described hereinabove, by removing a sidewall spacer (not shown) that may have been formed between first electrodes 1041a to 1043a and second electrodes 1041b to 1043b, and between first electrode-forming faces 1032a to 1032c and second electrode-forming faces 1033a to 1033c, in a contiguous manner, during a production process, nanogaps NG1, NG2, and NG3 may be formed between first electrodes 1041a to 1043a and second electrodes 1041b to 1043b, and at this time, channels 1037a to 1037c may also be formed between first electrode-forming faces 1032a to 1032c and second electrode-forming faces 1033a to 1033c.

In some cases for methods of production of composite nanogap electrode devices, the sidewall spacer may be removed, whereby channels 1037a to the 1037c may be formed so as to conform to a shape of said sidewall spacer between wall-like first electrodes 1041a to 1043a and wall like second electrodes 1041b to 1043b may be arranged to face each other while maintaining a constant distance therebetween, and nanogaps NG1, NG2, and NG3 may be formed contiguously with channels 1037a to 1037c on both sides of first electrodes 1041a to 1043a and second electrodes 1041b to 1043b respectively, so as to conform to a shape of said sidewall spacer with center axes O of channels 1037a to 1037c as centers. Accordingly, an object to be measured flowing in channels 1037a (1037b, 1037C) may pass along center axis O through channel 1037a (1037b, 1037c) and nanogap NG1 (NG2, NG3) without deviation between nanogap NG1 (NG2, NG3) and channels 1037a (1037b, 1037c), so that said object to be measured, flowing in channel 1037a (1037b, 1037c), may more easily pass through nanogap NG1 (NG2, NG3) than conventionally.

In some cases for methods or production of composite nanogap electrodes, wall-like sidewall spacer, which may be sandwiched between first electrodes 1041a to 1043a and second electrodes 1041b to 1043b, and which may also be sandwiched between first electrode-embedded layer 1032 and second electrode-embedded layer 1033, may be removed, so that nanogaps NG1, NG2, and NG3 may be formed at regions from which sidewall spacer 1014 between first electrodes 1041a to 1043a and second electrode 1041b to 1043b was removed, and that nanogap NG1, NG2, and NG3, which may be in fluid communication with channels 1037a to 1037c, may be formed at regions from which sidewall spacer between first electrode-embedded layer 1032 and second electrode-embedded layer 1033 was removed.

In some cases for methods of production of composite nanogap electrode devices as described hereinabove, channels 1037a, 1037b, and 1037c may be formed so as to conform to a shape of sidewall spacer, contiguous with wall-like first electrode-forming faces 1032a to 1032c and wall-like second electrode-forming faces 1033a to 1033c, which may be arranged to face each other while maintaining a constant distance therebetween, and nanogaps NG1, NG2, and NG3 may also be formed at the same time to be contiguous between wall-like first electrode side surface 1041c to 1043c and wall-like second electrode side surface 1041c to 1043d to conform to a shape of sidewall spacer 1014 across center axis O of channels 1037a, 1037b, and 1037c. Accordingly, composite nanogap electrode device 1031 may be manufactured whereby an object to be measured, flowing in channels 1037a, 1037b, and 1037c, may pass along a same center axis O through channel 1037a, 1037b, and 1037c and nanogaps NG1, NG2, and NG3, without deviation between nanogaps NG1, NG2, and NG3 and channels 1037a, 1037b, and 1037c, so that said object to be measured, flowing in channel 107, may more easily pass through nanogaps NG1, NG2, and NG3 than conventionally.

In some cases a nanochannel may be narrow near a nanogap electrode device and wider further away from said nanogap electrode device. In some cases a channel may taper down to a nanochannel so as to facilitate linearization of a biopolymer. A wider channel may reduce the risk of clogging by particles in the sample or due to fabrication defects. A narrow cross section near the chip may facilitate a higher percentage of the biopolymer being measured by a nanogap electrode device.

In some cases compound nanogap electrode devices may partly comprise sidewall spacer, which may have been formed in a contiguous manner between first electrode side surface 1041c of first electrode 1041a and second electrode side surface 1041d of second electrode side surface 1041d, and between first electrode-forming face 1032a and second electrode-forming face 1033a, may be removed, whereby nanogap NG1, which may be defined by wall-like electrode side surfaces arranged to face each other while maintaining a constant distance therebetween with center axis O of channel 1037a as a center, may be formed, which channel 1037a may be defined by wall-like first electrode-forming face 1032a and second electrode-forming face 1033a may be arranged to face each other while maintaining at a constant distance therebetween. Accordingly, an object to be measured, flowing in channel 1037a, may pass along a same center axis O through channel 1037 and nanogap NG1 without deviation between nanogap NG1 and channel 1037a, so that said object to be measured, flowing in channel 1037a, may more easily pass through nanogap NG1 than conventionally. For such a composite nanogap electrode device 1031, substantially similar measurements may be produced by other electrode devices 1034b and 1034c.

In some cases for composite nanogap electrode devices, other nanogap electrodes 1034b and 1034c may have a similar configuration as that of nanogap electrode device 1034a. Specifically, composite nanogap electrode device 1031 may be provided with nanogap NG2 (NG3) that may be formed by removing a side wall spacer, formed between first electrode side surface 1042c (1043c) of first electrode 1042a (1043a) and second electrode side surface 1042d (1043d) of second electrode 1042b (1043b), and between first electrode-forming face 1032b (1032c) and second electrode-forming face 1033b (1033c) in a contiguous manner, whereby nanogap NG2 (NG3), which may be defined by wall-like electrode side surfaces, which may be arranged to face each other while maintaining a constant distance therebetween with center axis O of channel 1037b (1037c) as a center, may be formed, which channel 1037b (1037c) may be defined by wall-like first electrode-forming face 1032b (1032c) and wall like second electrode-forming face 1033b (1033c) may be arranged to face each other while maintaining a constant distant therebetween. Accordingly, nanogap NG2 (NG3) and channel 1037b (1037c) may be less likely to deviate from each other, so that passing of an object to be measured through channel 1037b (1037c) and nanogap NG2 (NG3) along center axis O may be facilitated, and said object to be measured, flowing in channel 1037b (1037c), can more easily pass through nanogap NG1 than conventionally.

In some cases it may be desirable to have a smooth transition between a channel and a nanogap electrode device. In some cases, smooth transitions may allow electric field lines to be parallel to a center axis O when electrophoretic or electroosmotic flow forces may be used to move biomolecules in a linear direction past or through a nanogap electrode device. In some cases, a smooth transition may exist only on one side of a nanogap electrode device, so that, for example, a biopolymer may have a narrow channel on an outlet side so that a leading biopolymer end may be pulled through with more electrophoretic force per base than a corresponding tailing end of said biopolymer which may experience a lower electrophoretic force per base.

In some cases for composite nanogap electrode devices, one or more single-stranded DNA molecules, which may be an object to be measured contained in a solution, may be made to flow sequentially through three nanogaps NG1, NG2, and NG3, and said one or more single-stranded DNA molecules may be sequentially subjected to base sequence analysis at each of nanogap electrode devices 1034a, 1034b, and 1034c. Thus, said one or more single-stranded DNA molecules may be repeatedly subjected to base sequence analysis. Thus, even if an erroneous base sequence analysis result may be generated at one nanogap electrode device 1034a, said one or more single-stranded DNA molecule may be correctly analyzed based on base sequence analysis results generated at other nanogap electrode devices 1034b and 1034c.

In some cases a cover may be used to cap a top of a channel to constrain fluid. In some cases said cover may be attached planarized regions of a nanogap electrode device to cover a channel or nanochannel and nanogap electrode device by one of adhesive, covalent bonds, van der Walls force or physical clamping, wherein said planarization may aid with adhesion and with uniformity of fluidic flow and electrophoretic field strength.

In some cases for a composite nanogap electrode devices, similarly to nanogap electrodes as described hereinabove, a width of nanogap NG1 of nanogap electrode device 1034a, a width of the nanogap NG2 of nanogap electrode device 1034b, and a width of nanogap NG3 of nanogap electrode device 1034c may be freely selected by adjusting a film thickness of a sidewall spacer that may be used during a production process. Thus, by very appropriately selecting these nanogaps NG1, NG2, and NG3, one or more single-stranded DNA molecules may be analyzed with high sensitivity.

In some cases for methods of production of composite nanogap electrode devices, similarly to the production methods described hereinabove for nanogap electrode devices, a sidewall spacer that may be provided in an erect manner between first electrode-embedded layer 1032 and second electrode-embedded layer 1033, sidewall spacer between first electrodes 1041a, 1042a, 1043a and second electrodes 1041b, 1042b, 1043b, and sidewall between first electrode-embedded layer 1032 and second electrode-embedded layer 1033, may be removed at the same time, whereby nanogaps NG1, NG2, and NG3 may be formed between first electrodes 1041a, 1042a, 1043a and second electrodes 1041b, 1042b, 1043b, respectively. Therefore, a production process may be simplified compared to a case of independently forming a nanogap and a channel.

Alternatively or additionally, first electrode 1010 (1041a to 1043a, 1052), second electrode 1011 (1041b to 1043b, 1053), sidewall spacer 1014 (105, 1044, 1058a), first electrode-embedded layer 103 (1032, 1047), and second electrode-embedded layer 104 (1033, 1049), etc., may be formed of any of various materials. Furthermore, first electrode 1010 (1041a to 1043a, 1052), second electrode 1011 (1041b to 1043b, 1053), channels 107 (1037a to 1037c, 1051), solution supply part 108 (1038a), solution discharge part 109 (1036d), and solution-passing part (1036b, 1036c), etc., of any of the cases described herein may be formed to have any of various shapes. For example, in some cases, channel 107 may be formed by etching off an entirety of sidewall spacer 1014. Alternatively or additionally, channel 107 may be formed to have shallower depth, or it may be formed to have greater depth by removing not only sidewall spacer 1014 but also first electrode-embedded layer 103 therebelow, by controlling etching conditions for sidewall spacer 1014.

In some cases described hereinabove for nanogap electrode devices 101 (41b to 1043b, 1053) is described for which one or more single-stranded DNA molecules may be caused to pass through nanogap NG (NG1, NG2, NG3) between first electrode 1010 (1041a to 1043a, 1052) and second electrode 1011 (1041b to 1043b, 1053), and in which an ammeter may be caused to measure values of current(s) flowing through first electrode 1010 (1041a to 1043a, 1052) and second electrode 1011 (1041b to 1043b, 1053) when each base said one or more single-stranded DNA molecules passes through nanogap NG (NG1, NG2, NG3) between first electrode 1010 (1041a to 1043a, 1052) and second electrode 1011 (1041b to 1043b, 1053). Alternatively or additionally, nanogap electrodes may be used for measuring a current value for any of various objects to be measured, for example, a biopolymer such as RNA, proteins, carbohydrates, lipids, double stranded DNA molecules, partially double stranded DNA molecules, labeled DNA molecules, wherein said label may be an organic or inorganic label as well as said one or more single stranded DNA molecules; said one or more single stranded DNA molecules may comprise standard DNA bases, abasic DNA bases, naturally or synthetically modified DNA bases, natural DNA, synthetic DNA, RNA, modified RNA, chimerically bound proteins, carbohydrates, or other organic or inorganic molecules.

In some cases as described hereinabove, an example is given in which sidewall spacer 1014, which may be formed to gradually increase in width from a top thereof to a bottom face which adjacent to substrate 2, may be used. Alternatively or additionally, a sidewall spacer-forming layer may not be formed in a conformal manner. A side wall spacer-forming layer may be formed to have different film thicknesses at different locations by changing the film deposition conditions (such as temperature, pressure, applied gas, flow rate, etc.). It may also be possible to use a sidewall spacer that may be formed so as to gradually decrease in width from a top to a bottom adjacent to substrate 2, or a sidewall spacer formed to have a maximum width at various portions, for example, at a top position, at a center position, etc.

Furthermore, in some cases of composite nanogap electrode device 1031 for which three nanogap electrode devices 1034a, 1034b, and 1034c may be formed on electrode device-forming substrate 1035. Alternatively or additionally, the number of nanogap electrodes, positions at which said nanogap electrode devices may be disposed, and the number of solution-passing parts may be changed as appropriate. For example, at least two nanogap electrodes devices may be arranged associated with a single channel having no solution-passing part. In other words, in some cases, channels may fluidically communicate via a solution-passing part. However, channels may also be directly connected to fluidically communicate with each other.

Channels may be connected so that at least two nanogap electrode devices may be linearly arranged. In this case, channels may be directly connected to fluidically communicate with each other, or may be connected via a solution-passing part.

In some cases, a composite nanogap electrode device 1031 is described which may be provided with second electrode-embedded layer 1033 that may be formed directly on first electrode-embedded layer 1032. Alternatively or additionally, a composite nanogap electrode device may be provided with second electrode-embedded layer 1033 that may be formed in a recess in first electrode-embedded layer 1032 via a lower spacer.

In other cases, a biopolymer analyzing apparatus including a nanogap electrode device as described above, and a biopolymer analyzing system including such a nanogap electrode device or including such a biopolymer analyzing apparatus may be utilized.

In some cases as described hereinabove for nanogap electrode devices, a biopolymer analyzing apparatus may further include a power supply part for supplying a current to a first electrode and a second electrode of a nanogap electrode device, and an amplification part for amplifying currents that flow through a first electrode and a second electrode. A biopolymer analyzing apparatus may include an information processing part for analyzing an amplified electric signal. An information processing part may include one or more CPUs (Central Processing Unit) or computers. Furthermore, a biopolymer analyzing apparatus may include one or more memory parts (storage). A memory part (storage) may store an obtained electric signal, an amplified electric signal, analyzed information, and various other information. Furthermore, a biopolymer analyzing apparatus may include an electric shield and or vibration isolation, to reduce or eliminate electric noise or mechanical noise inside and outside thereof. In some cases, a biopolymer analyzing apparatus may be a DNA or RNA sequencer. A biopolymer analyzing apparatus may cause a biopolymer to pass through a nanogap between a first electrode and a second electrode through a channel formed in a nanogap electrode device, and may analyze a biopolymer based on a change of current flowing through said first and second electrodes.

In some cases for a biopolymer analyzing apparatus, a nanogap may be formed by forming first electrode-forming face and second-electrode forming face arranged to face each other while maintaining a constant distance therebetween, forming a channel that extends along a center axis thereof, and arranging said first electrode side surface of a first electrode and said second electrode side surface of a second electrode with said center axis of said channel as a center. As a result, a biopolymer analyzing apparatus may direct passing of a biopolymer, which may be an object to be measured, through a channel to a nanogap along a center axis O, and said biopolymer may be measured, while flowing in said channel, and said biopolymer may more easily pass through said nanogap NG1 than conventionally.

In a biopolymer analyzing system according to some cases, a nanogap electrode device or a biopolymer analyzing apparatus as described above may be connected with another component or another part wirelessly or by wire. Such a component or a part may be any component or any part described in connection with cases of biopolymer analyzing apparatus' as described hereinabove. For example, a biopolymer analyzing system may include an information processing apparatus for performing various information processes on analysis results obtained from said biopolymer analyzing apparatus. In some cases of biopolymer analyzing system(s), for example, said biopolymer analyzing apparatus and information processing apparatus may be connected by wire to receive and transmit data therebetween via wire. Furthermore, for said biopolymer analyzing system, for example, said biopolymer analyzing apparatus and said information processing apparatus may be installed and configured on a premise of a research laboratory, etc. As a result, biopolymer analysis results obtained by said biopolymer analyzing apparatus may be transmitted to an information processing apparatus located on the same premises, so that various information processes such as comparison of multiple analysis results by the information processing apparatus, and calculation of statistical data of analysis results, can be effectively performed.

In some cases, a biopolymer analyzing system may be configured, for example, by wirelessly connecting a biopolymer analyzing apparatus and an information processing apparatus over a network such as the internet. As a result, said biopolymer analyzing apparatus installed within a facility such as a hospital may transmit and receive data with said information processing apparatus that may be installed in one or more different remote sites from that of the facility at which said biopolymer analyzing apparatus may be installed.

For such a biopolymer analyzing system, biopolymer analysis results obtained by said biopolymer analyzing apparatus may be transmitted to said information processing apparatus at one or more different remote sites from the site at which said biopolymer analyzing apparatus may be installed, so that various information processes such as comparison of multiple analysis results by said information processing apparatus, and calculation of statistic data of analysis results, can be effectively performed.

In some cases, said biopolymer analysis apparatus may be a portable apparatus, and may not be installed a fixed facility, but may instead utilize wireless transmission of data to move data generated by said biopolymer analysis apparatus to said information processing apparatus, which may be located at one or more remote sites.

Furthermore, such a biopolymer analyzing system may be provided with a nanogap electrode device according to any of the cases described hereinabove or may be provided with a biopolymer analyzing apparatus including such a nanogap electrode device. Thus, passing of a biopolymer, which may be an object to be measured, through a channel to a nanogap, is facilitated, and thus, said object to be measured flowing in a channel may pass through a nanogap NG more easily, and analysis results may be obtained accurately and more effectively than conventionally.

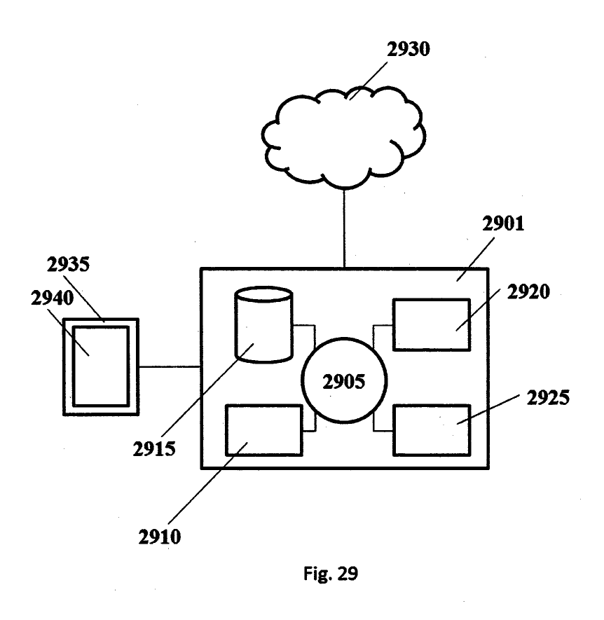

The present disclosure provides computer control systems that are programmed to implement methods of the disclosure. FIG. 29 shows a computer system 2901 that is programmed or otherwise configured to fabricate electrodes for use in sensing biomolecules. The computer system 2901 can regulate various aspects of methods of the present disclosure, such as, for example, the formation of various device layers.

The computer system 2901 includes a central processing unit (CPU, also “processor” and “computer processor” herein) 2905, which can be a single core or multi core processor, or a plurality of processors for parallel processing. The computer system 2901 also includes memory or memory location 2910 (e.g., random-access memory, read-only memory, flash memory), electronic storage unit 2915 (e.g., hard disk), communication interface 2920 (e.g., network adapter) for communicating with one or more other systems, and peripheral devices 2925, such as cache, other memory, data storage and/or electronic display adapters. The memory 2910, storage unit 2915, interface 2920 and peripheral devices 2925 are in communication with the CPU 2905 through a communication bus (solid lines), such as a motherboard. The storage unit 2915 can be a data storage unit (or data repository) for storing data. The computer system 2901 can be operatively coupled to a computer network (“network”) 2930 with the aid of the communication interface 2920. The network 2930 can be the Internet, an internet and/or extranet, or an intranet and/or extranet that is in communication with the Internet. The network 2930 in some cases is a telecommunication and/or data network. The network 2930 can include one or more computer servers, which can enable distributed computing, such as cloud computing. The network 2930, in some cases with the aid of the computer system 2901, can implement a peer-to-peer network, which may enable devices coupled to the computer system 2901 to behave as a client or a server.

The CPU 2905 can execute a sequence of machine-readable instructions, which can be embodied in a program or software. The instructions may be stored in a memory location, such as the memory 2910. The instructions can be directed to the CPU 2905, which can subsequently program or otherwise configure the CPU 2905 to implement methods of the present disclosure. Examples of operations performed by the CPU 2905 can include fetch, decode, execute, and writeback.

The CPU 2905 can be part of a circuit, such as an integrated circuit. One or more other components of the system 2901 can be included in the circuit. In some cases, the circuit is an application specific integrated circuit (ASIC).

The storage unit 2915 can store files, such as drivers, libraries and saved programs. The storage unit 2915 can store user data, e.g., user preferences and user programs. The computer system 2901 in some cases can include one or more additional data storage units that are external to the computer system 2901, such as located on a remote server that is in communication with the computer system 2901 through an intranet or the Internet. The computer system 2901 can communicate with one or more remote computer systems through the network 2930.

Methods as described herein can be implemented by way of machine (e.g., computer processor) executable code stored on an electronic storage location of the computer system 2901, such as, for example, on the memory 2910 or electronic storage unit 2915. The machine executable or machine readable code can be provided in the form of software. During use, the code can be executed by the processor 2905. In some cases, the code can be retrieved from the storage unit 2915 and stored on the memory 2910 for ready access by the processor 2905. In some situations, the electronic storage unit 2915 can be precluded, and machine-executable instructions are stored on memory 2910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

The computer system 2901 can be programmed or otherwise configured to regulate one or more processing parameters, such as the substrate temperature, precursor flow rates, growth rate, carrier gas flow rate and reaction chamber pressure. The computer system 2901 can be in communication with valves between the storage vessels and a reaction chamber, which can aid in terminating (or regulating) the flow of a precursor to the reaction chamber.

The computer system 2901 can be in communication with a vacuum system comprising a vacuum chamber, flow valves and a pumping system. The vacuum system can include one or more vacuum pumps, such as one or more of a turbomolecular (“turbo”) pump, a diffusion pump and a mechanical pump. A pump may include one or more backing pumps. For example, a turbo pump may be backed by a mechanical pump.

Aspects of the systems and methods provided herein, such as the computer system 2901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Methods and systems of the present disclosure can be implemented by way of one or more algorithms. An algorithm can be implemented by way of software upon execution by the central processing unit 2905.

Devices, systems and methods of the present disclosure may be combined with and/or modified by other devices, systems, or methods, such as those described in, for example, US 2002/0168810, US 2010/0025249, US 2012/0193237, US 2012/0322055, US 2013/0001082, US 2014/0300339, US 2014/0302675, JP 2005-257687A, JP 2008-32529A, JP 2011-163934A, JP 2011-163934A and JP 2013-36865A, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A device for detecting a biopolymer, comprising:
a channel that is configured to direct said biopolymer, wherein a width of said channel is less than 10 nanometers (nm); and
a pair of electrodes in a portion of said channel, wherein said pair of electrodes have surfaces that are substantially coplanar with adjacent surfaces of said channel, which surfaces of said pair of electrodes are exposed during use of said device to enable detection of said biopolymer or a portion thereof with the aid of said pair of electrodes.

2. (canceled)

3. (canceled)

4. (canceled)

5. The device of claim 1, wherein said pair of electrode include tips separated by a gap, which gap has a spacing that is less than said width.

6. The device of claim 5, wherein said spacing is from 0.5 to 2 times a molecular diameter of said biopolymer.

7. The device of claim 6, wherein said spacing is from 0.5 to less than a molecular diameter of said biopolymer.

8. The device of claim 1, further comprising a control system in electrical communication with said pair of electrodes, wherein said control system (i) receives signals from said pair of electrodes and (ii) uses said signals to detect said biopolymer or a portion thereof.

9. The device of claim 1, wherein said channel includes multiple pairs of electrodes with surface that are coplanar with adjacent surfaces of said channel.

10. The device of claim 1, wherein said pair of electrodes has a gap that is within 2 nm of said width.

11. A device for biopolymer detection, comprising:
a first electrode-embedded layer comprising an insulating material, said first electrode-embedded layer having a first electrode-forming face;
a second electrode-embedded layer comprising an insulating material, said second electrode-embedded layer having a second electrode-forming face that faces said first electrode-forming face;
a first electrode and a second electrode, wherein said first electrode has a first electrode side surface that is exposed within said first electrode-forming face, and wherein said second electrode has a second electrode side surface that is exposed within said second electrode-forming face; and
a channel that is at least partially defined by said first electrode-forming face and said second electrode-forming face, wherein said channel (i) extends along a center line between said first electrode-forming face and said second electrode-forming face and (ii) has a width that is substantially constant,
wherein said first electrode side surface and said second electrode side surface are disposed in at most a portion of said channel, and
wherein said first electrode side surface and second electrode side surface are spaced apart by a gap that has a distance that is substantially the same as said width.

12. The device of claim 11, wherein said first electrode-forming face and said first electrode side surface are contiguous.

13. The device of claim 11, wherein said second electrode-forming face and said second electrode side surface are contiguous.

14. The device of claim 11, wherein said width is less than 10 nanometers.

15. The device of claim 11, wherein said gap is substantially within 2 nanometers of said width.

16. The device of claim 11, wherein said channel is band-like.

17. The device of claim 11, wherein said channel is substantially straight or curved.

18. The device of claim 11, wherein said gap is disposed between ends of said channel.

19. The device of claim 11, further comprising a fluid supply member and a fluid discharge member in fluid communication with said channel, wherein each of said fluid supply member and fluid discharge member has a width greater than said width of said channel.

20. The device of claim 11, wherein said second electrode-embedded layer is on a lower spacer layer.

21.-50. (canceled)

51. The device of claim 1, wherein said detection of said biopolymer or a portion thereof is based on electrical signals measured using said pair of electrodes.

52. The device of claim 11, further comprising a control system that (i) receives signals from said first electrode and said second electrode as a biomolecule passes through said channel between said first electrode and said second electrode, and (ii) uses said signals to detect or analyze a biopolymer or a portion thereof.

53. The device of claim 52, wherein said signals are electrical signals.
